SEQUENCE LISTING (65 sequences)

SEQ ID NO: 1 - protein, 508 aa, Artificial Sequence
  CAR fragment
AQSFGLLDPKLCYLLDGILFIYRVILTALFLRATNFSLLKQAGDVEENPGPMALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSKRSRLLHSDYMNMTPRRPGPTRKHYQPYAPPRDFAAYRSRVK

SEQ ID NO: 2 - protein, 32 aa, Artificial Sequence
  CAR component
AQSFGLLDPKLCYLLDGILFIYRVILTALFLR

SEQ ID NO: 3 - protein, 19 aa, Artificial Sequence
  CAR component
ATNFSLLKQAGDVEENPGP

SEQ ID NO: 4 - protein, 21 aa, Artificial Sequence
  CAR component
MALPVTALLLPLALLLHAARP

SEQ ID NO: 5 - protein, 107 aa, Artificial Sequence
  CAR component
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 6 - protein, 15 aa, Artificial Sequence
  CAR component
GGGGSGGGGSGGGGS

SEQ ID NO: 7 - protein, 120 aa, Artificial Sequence
  CAR component
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 8 - protein, 12 aa, Artificial Sequence
  CAR component
ESKYGPPCPPCP

SEQ ID NO: 9 - protein, 107 aa, Artificial Sequence
  CAR component
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 10 - protein, 4 aa, Artificial Sequence
  CAR component
KDPK

SEQ ID NO: 11 - protein, 27 aa, Homo sapiens
FWVLVVVGGVLACYSLLVTVAFIIFWV

SEQ ID NO: 12 - protein, 41 aa, Homo sapiens
RSKRSRLLHSDYMNMTPRRPGPTRKHYQPYAPPRDFAAYRS

SEQ ID NO: 13 - protein, 3 aa, Artificial Sequence
  CAR component
RVK

SEQ ID NO: 14 - protein, 617 aa, Artificial Sequence
  CAR fragment
AQSFGLLDPKLCYLLDGILFIYRVILTALFLRATNFSLLKQAGDVEENPGPMALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 505 aa, Artificial Sequence
  CAR fragment
FSRSADAPAYATNFSLLKQAGDVEENPGPMALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYN

SEQ ID NO: 16 - protein, 10 aa, Artificial Sequence
  CAR component
FSRSADAPAY

SEQ ID NO: 17 - protein, 473 aa, Artificial Sequence
  CAR component
ATNFSLLKQAGDVEENPGPMALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSKRSRLLHSDYMNMTPRRPGPTRKHYQPYAPPRDFAAYRS

SEQ ID NO: 18 - protein, 8 aa, Artificial Sequence
  CAR component
RVKFSRSA

SEQ ID NO: 19 - protein, 14 aa, Artificial Sequence
  CAR component
DAPAYQQGQNQLYN

SEQ ID NO: 20 - protein, 595 aa, Artificial Sequence
  CAR fragment
FSRSADAPAYATNFSLLKQAGDVEENPGPMALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 239 aa, Artificial Sequence
  enhanced GFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 23 - protein, 25 aa, Artificial Sequence
  CAR component
IQNPDPAVYQLRDSKSSDKSVCLFT

SEQ ID NO: 24 - DNA, 2221 bp, Artificial Sequence
  CAR fragment
ggcaggagctctgtgcacagagaacagcctcatctgctcgccttgtttccacctcccctcccattgccccaggttctttggccccacagcggccacatctgccgttggtgccaataggttttccaggagctggttgaggtgggagggagggagagggttgtgatcaggctgaggcatggggattggatatagtctccgtgtcatgatttatttggtcagtcagtcctagtgccaccctggggtaatggggatgtgttctcgtcaccttgggcctggctgaccagctttatctcttggcacagaggcacagagctttggcctgctggatcccaaactctgctacctgctggatggaatcctcttcatctatcgtgtcattctcactgccttgttcctgagagccaccaacttctctcttttgaagcaggccggagatgtggaagaaaatcctgggcccatggctcttcctgtgactgcccttctgctgcccctggctctcctgctccatgctgcccggccagacatccagatgacacagacaaccagcagcctcagcgccagcctgggggacagagtgaccattagctgccgggcctctcaggacatcagcaaatacctgaactggtaccagcagaaaccagatggcactgtcaagctgctgatttaccacacctccaggctccacagcggcgtgcccagtcgcttcagcggcagtgggagcgggacagattattccctcacaatctccaacctggagcaggaagatattgccacatacttctgccagcaaggcaacaccctgccatacacatttggaggcggcaccaaattggagatcaccggcggtggtggatctggaggagggggcagcggaggtggcggctctgaggtgaaactgcaggagagtggccctggcctggtggctcccagccagagcctttctgtcacctgcaccgtgtctggggtgtccctgcctgactatggagtctcctggatccggcagcctccaagaaaaggactggaatggctgggcgtcatctggggaagtgagaccacctactataattcagccctcaagtcccggctcaccatcattaaggacaactccaaatcccaggtgttcctgaagatgaattctctccagactgatgacacagccatctactactgtgccaagcattattattacggcgggtcctatgccatggactactggggccaggggaccagtgtcactgtttcttctgaaagcaagtatggccccccatgccctccctgccccggacagccgcgggagccccaggtctacaccctcccaccctccagagatgagctgaccaagaaccaggtttcactgacatgcctggtgaagggcttctacccctctgacattgctgtggagtgggagagcaatgggcagccagagaacaactacaagaccacacctcctgtcctggacagcgacggctccttcttcctctacagtaagctcactgtggacaagagccgctggcagcagggcaatgtcttctcctgcagcgtgatgcacgaggccctgcacaatgcctatacccagaagtcactctctctgagccctggaaagaaggatcccaagttttgggtcctcgtggtggtcgggggtgtgctggcctgctacagcttgctggtcacagtggccttcatcatcttctgggtgcgctccaagaggagccggctgcttcacagtgattacatgaacatgacccccaggaggccaggacccaccaggaagcactaccagccctacgctcccccgcgggactttgctgcttaccgcagcagagtgaaggtgggtaccactgggctttgggaggagggcacggggtcccccacttgatggatgttcagaggggccttggtcttggaaggtctcaagctcgggtggtgcctggggcttggtatccaggagcaaagcaaggaccagccaagtgtgtgccttgagtgggctgaggaggaggtggcagtgtctggctgagatggacagggtaggagggagagcctggtgctaggcacctccatgacaagccgtacaaatgtgtgcacatcagagtgtcccagggaaggcgatgctactggtgacaaaggggcttacactcaggcagaggtccttctttccaagtgtgaatgaaggccatgttagcctttctcttgaaaaggccctttcctcatctgtaactgggga

SEQ ID NO: 25 - DNA, 400 bp, Artificial Sequence
  CAR construct
ggcaggagctctgtgcacagagaacagcctcatctgctcgccttgtttccacctcccctcccattgccccaggttctttggccccacagcggccacatctgccgttggtgccaataggttttccaggagctggttgaggtgggagggagggagagggttgtgatcaggctgaggcatggggattggatatagtctccgtgtcatgatttatttggtcagtcagtcctagtgccaccctggggtaatggggatgtgttctcgtcaccttgggcctggctgaccagctttatctcttggcacagaggcacagagctttggcctgctggatcccaaactctgctacctgctggatggaatcctcttcatctatcgtgtcattctcactgccttgttcctgaga

SEQ ID NO: 26 - DNA, 57 bp, Artificial Sequence
  CAR component
gccaccaacttctctcttttgaagcaggccggagatgtggaagaaaatcctgggccc

SEQ ID NO: 27 - DNA, 63 bp, Artificial Sequence
  CAR component
atggctcttcctgtgactgcccttctgctgcccctggctctcctgctccatgctgcccggcca

SEQ ID NO: 28 - DNA, 321 bp, Artificial Sequence
  CAR component
gacatccagatgacacagacaaccagcagcctcagcgccagcctgggggacagagtgaccattagctgccgggcctctcaggacatcagcaaatacctgaactggtaccagcagaaaccagatggcactgtcaagctgctgatttaccacacctccaggctccacagcggcgtgcccagtcgcttcagcggcagtgggagcgggacagattattccctcacaatctccaacctggagcaggaagatattgccacatacttctgccagcaaggcaacaccctgccatacacatttggaggcggcaccaaattggagatcacc

SEQ ID NO: 29 - DNA, 45 bp, Artificial Sequence
  CAR component
ggcggtggtggatctggaggagggggcagcggaggtggcggctct

SEQ ID NO: 30 - DNA, 360 bp, Artificial Sequence
  CAR component
gaggtgaaactgcaggagagtggccctggcctggtggctcccagccagagcctttctgtcacctgcaccgtgtctggggtgtccctgcctgactatggagtctcctggatccggcagcctccaagaaaaggactggaatggctgggcgtcatctggggaagtgagaccacctactataattcagccctcaagtcccggctcaccatcattaaggacaactccaaatcccaggtgttcctgaagatgaattctctccagactgatgacacagccatctactactgtgccaagcattattattacggcgggtcctatgccatggactactggggccaggggaccagtgtcactgtttcttct

SEQ ID NO: 31 - DNA, 36 bp, Artificial Sequence
  CAR component
gaaagcaagtatggccccccatgccctccctgcccc

SEQ ID NO: 32 - DNA, 321 bp, Artificial Sequence
  CAR component
ggacagccgcgggagccccaggtctacaccctcccaccctccagagatgagctgaccaagaaccaggtttcactgacatgcctggtgaagggcttctacccctctgacattgctgtggagtgggagagcaatgggcagccagagaacaactacaagaccacacctcctgtcctggacagcgacggctccttcttcctctacagtaagctcactgtggacaagagccgctggcagcagggcaatgtcttctcctgcagcgtgatgcacgaggccctgcacaatgcctatacccagaagtcactctctctgagccctggaaag

SEQ ID NO: 33 - DNA, 12 bp, Artificial Sequence
  CAR component
aaggatcccaag

SEQ ID NO: 34 - DNA, 81 bp, Homo sapiens
ttttgggtcctcgtggtggtcgggggtgtgctggcctgctacagcttgctggtcacagtggccttcatcatcttctgggtg

SEQ ID NO: 35 - DNA, 123 bp, Homo sapiens
cgctccaagaggagccggctgcttcacagtgattacatgaacatgacccccaggaggccaggacccaccaggaagcactaccagccctacgctcccccgcgggactttgctgcttaccgcagc

SEQ ID NO: 36 - DNA, 402 bp, Artificial Sequence
  CAR construct
agagtgaaggtgggtaccactgggctttgggaggagggcacggggtcccccacttgatggatgttcagaggggccttggtcttggaaggtctcaagctcgggtggtgcctggggcttggtatccaggagcaaagcaaggaccagccaagtgtgtgccttgagtgggctgaggaggaggtggcagtgtctggctgagatggacagggtaggagggagagcctggtgctaggcacctccatgacaagccgtacaaatgtgtgcacatcagagtgtcccagggaaggcgatgctactggtgacaaaggggcttacactcaggcagaggtccttctttccaagtgtgaatgaaggccatgttagcctttctcttgaaaaggccctttcctcatctgtaactgggga

SEQ ID NO: 37 - DNA, 2812 bp, Artificial Sequence
  CAR fragment
ggcaggagctctgtgcacagagaacagcctcatctgctcgccttgtttccacctcccctcccattgccccaggttctttggccccacagcggccacatctgccgttggtgccaataggttttccaggagctggttgaggtgggagggagggagagggttgtgatcaggctgaggcatggggattggatatagtctccgtgtcatgatttatttggtcagtcagtcctagtgccaccctggggtaatggggatgtgttctcgtcaccttgggcctggctgaccagctttatctcttggcacagaggcacagagctttggcctgctggatcccaaactctgctacctgctggatggaatcctcttcatctatcgtgtcattctcactgccttgttcctgagagccaccaacttctctcttttgaagcaggccggagatgtggaagaaaatcctgggcccatggctcttcctgtgactgcccttctgctgcccctggctctcctgctccatgctgcccggccagacatccagatgacacagacaaccagcagcctcagcgccagcctgggggacagagtgaccattagctgccgggcctctcaggacatcagcaaatacctgaactggtaccagcagaaaccagatggcactgtcaagctgctgatttaccacacctccaggctccacagcggcgtgcccagtcgcttcagcggcagtgggagcgggacagattattccctcacaatctccaacctggagcaggaagatattgccacatacttctgccagcaaggcaacaccctgccatacacatttggaggcggcaccaaattggagatcaccggcggtggtggatctggaggagggggcagcggaggtggcggctctgaggtgaaactgcaggagagtggccctggcctggtggctcccagccagagcctttctgtcacctgcaccgtgtctggggtgtccctgcctgactatggagtctcctggatccggcagcctccaagaaaaggactggaatggctgggcgtcatctggggaagtgagaccacctactataattcagccctcaagtcccggctcaccatcattaaggacaactccaaatcccaggtgttcctgaagatgaattctctccagactgatgacacagccatctactactgtgccaagcattattattacggcgggtcctatgccatggactactggggccaggggaccagtgtcactgtttcttctgaaagcaagtatggccccccatgccctccctgccccggacagccgcgggagccccaggtctacaccctcccaccctccagagatgagctgaccaagaaccaggtttcactgacatgcctggtgaagggcttctacccctctgacattgctgtggagtgggagagcaatgggcagccagagaacaactacaagaccacacctcctgtcctggacagcgacggctccttcttcctctacagtaagctcactgtggacaagagccgctggcagcagggcaatgtcttctcctgcagcgtgatgcacgaggccctgcacaatgcctatacccagaagtcactctctctgagccctggaaagaaggatcccaagttttgggtcctcgtggtggtcgggggtgtgctggcctgctacagcttgctggtcacagtggccttcatcatcttctgggtgcgctccaagaggagccggctgcttcacagtgattacatgaacatgacccccaggaggccaggacccaccaggaagcactaccagccctacgctcccccgcgggactttgctgcttaccgcagcagggtcaaattttctagatctgcagatgcgccggcctatcaacaaggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaataagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcagagtgaaggtgggtaccactgggctttgggaggagggcacggggtcccccacttgatggatgttcagaggggccttggtcttggaaggtctcaagctcgggtggtgcctggggcttggtatccaggagcaaagcaaggaccagccaagtgtgtgccttgagtgggctgaggaggaggtggcagtgtctggctgagatggacagggtaggagggagagcctggtgctaggcacctccatgacaagccgtacaaatgtgtgcacatcagagtgtcccagggaaggcgatgctactggtgacaaaggggcttacactcaggcagaggtccttctttccaagtgtgaatgaaggccatgttagcctttctcttgaaaaggccctttcctcatctgtaactgggga

SEQ ID NO: 38 - DNA, 2258 bp, Artificial Sequence
  CAR fragment
attgtcctcagagtccccggaggaagggatcctagagaatactcatgtccccgggggcgcagaccaagaaccttatacctctgcgcacgcgcgagggcgctagcccgggaagaataaactccagcgggtcctgggccagcgtgatgaatgcacgtgtcacaggcgggaggaaaaaggacaactgggctcagaaaaaggggctgccacgccccggcccctgcaagggctcagccccaggaggcagggcagcattgttagttgccaaggagcggagtagggctgtccctcgggtcgccgcgcgccctggcaccccggctcccctgcgcgccctcgcggagcagtaacgcgctttgctttctgtgttgcagttcagcaggagcgcagacgcccccgcgtacgccaccaacttctctcttttgaagcaggccggagatgtggaagaaaatcctgggcccatggctcttcctgtgactgcccttctgctgcccctggctctcctgctccatgctgcccggccagacatccagatgacacagacaaccagcagcctcagcgccagcctgggggacagagtgaccattagctgccgggcctctcaggacatcagcaaatacctgaactggtaccagcagaaaccagatggcactgtcaagctgctgatttaccacacctccaggctccacagcggcgtgcccagtcgcttcagcggcagtgggagcgggacagattattccctcacaatctccaacctggagcaggaagatattgccacatacttctgccagcaaggcaacaccctgccatacacatttggaggcggcaccaaattggagatcaccggcggtggtggatctggaggagggggcagcggaggtggcggctctgaggtgaaactgcaggagagtggccctggcctggtggctcccagccagagcctttctgtcacctgcaccgtgtctggggtgtccctgcctgactatggagtctcctggatccggcagcctccaagaaaaggactggaatggctgggcgtcatctggggaagtgagaccacctactataattcagccctcaagtcccggctcaccatcattaaggacaactccaaatcccaggtgttcctgaagatgaattctctccagactgatgacacagccatctactactgtgccaagcattattattacggcgggtcctatgccatggactactggggccaggggaccagtgtcactgtttcttctgaaagcaagtatggccccccatgccctccctgccccggacagccgcgggagccccaggtctacaccctcccaccctccagagatgagctgaccaagaaccaggtttcactgacatgcctggtgaagggcttctacccctctgacattgctgtggagtgggagagcaatgggcagccagagaacaactacaagaccacacctcctgtcctggacagcgacggctccttcttcctctacagtaagctcactgtggacaagagccgctggcagcagggcaatgtcttctcctgcagcgtgatgcacgaggccctgcacaatgcctatacccagaagtcactctctctgagccctggaaagaaggatcccaagttttgggtcctcgtggtggtcgggggtgtgctggcctgctacagcttgctggtcacagtggccttcatcatcttctgggtgcgctccaagaggagccggctgcttcacagtgattacatgaacatgacccccaggaggccaggacccaccaggaagcactaccagccctacgctcccccgcgggactttgctgcttaccgcagcagggtcaaattttctagatctgcagatgcgccggcctatcaacaaggccagaaccagctctataacgtaagtcagcctcgccgtagaccctccggaaaggaagggcagcctcctcctcggacctagggaacgcgcccgccagagtcttgggtttaggtttggtccctagctatcacctctgcgacccggctgcaacttgtttaacctcagttttctaacccacaaaacaggtgtagtaattgtacctgtctcaaagggcgccataaagattacatgctttaatgtcttgatgcatggaacgagcttagtctagtgcctgccactagtgagcccccaagccatcagcaatttgcaaaggctagaggggggtggagaaggaatgtattagaatcttcccagaggaggggaaagcgatttcacactccctgtgcaatgggactg

SEQ ID NO: 39 - DNA, 398 bp, Artificial Sequence
  CAR construct
attgtcctcagagtccccggaggaagggatcctagagaatactcatgtccccgggggcgcagaccaagaaccttatacctctgcgcacgcgcgagggcgctagcccgggaagaataaactccagcgggtcctgggccagcgtgatgaatgcacgtgtcacaggcgggaggaaaaaggacaactgggctcagaaaaaggggctgccacgccccggcccctgcaagggctcagccccaggaggcagggcagcattgttagttgccaaggagcggagtagggctgtccctcgggtcgccgcgcgccctggcaccccggctcccctgcgcgccctcgcggagcagtaacgcgctttgctttctgtgttgcagttcagcaggagcgcagacgcccccgcgtac

SEQ ID NO: 40 - DNA, 1419 bp, Artificial Sequence
  CAR body
gccaccaacttctctcttttgaagcaggccggagatgtggaagaaaatcctgggcccatggctcttcctgtgactgcccttctgctgcccctggctctcctgctccatgctgcccggccagacatccagatgacacagacaaccagcagcctcagcgccagcctgggggacagagtgaccattagctgccgggcctctcaggacatcagcaaatacctgaactggtaccagcagaaaccagatggcactgtcaagctgctgatttaccacacctccaggctccacagcggcgtgcccagtcgcttcagcggcagtgggagcgggacagattattccctcacaatctccaacctggagcaggaagatattgccacatacttctgccagcaaggcaacaccctgccatacacatttggaggcggcaccaaattggagatcaccggcggtggtggatctggaggagggggcagcggaggtggcggctctgaggtgaaactgcaggagagtggccctggcctggtggctcccagccagagcctttctgtcacctgcaccgtgtctggggtgtccctgcctgactatggagtctcctggatccggcagcctccaagaaaaggactggaatggctgggcgtcatctggggaagtgagaccacctactataattcagccctcaagtcccggctcaccatcattaaggacaactccaaatcccaggtgttcctgaagatgaattctctccagactgatgacacagccatctactactgtgccaagcattattattacggcgggtcctatgccatggactactggggccaggggaccagtgtcactgtttcttctgaaagcaagtatggccccccatgccctccctgccccggacagccgcgggagccccaggtctacaccctcccaccctccagagatgagctgaccaagaaccaggtttcactgacatgcctggtgaagggcttctacccctctgacattgctgtggagtgggagagcaatgggcagccagagaacaactacaagaccacacctcctgtcctggacagcgacggctccttcttcctctacagtaagctcactgtggacaagagccgctggcagcagggcaatgtcttctcctgcagcgtgatgcacgaggccctgcacaatgcctatacccagaagtcactctctctgagccctggaaagaaggatcccaagttttgggtcctcgtggtggtcgggggtgtgctggcctgctacagcttgctggtcacagtggccttcatcatcttctgggtgcgctccaagaggagccggctgcttcacagtgattacatgaacatgacccccaggaggccaggacccaccaggaagcactaccagccctacgctcccccgcgggactttgctgcttaccgcagc

SEQ ID NO: 41 - DNA, 24 bp, Artificial Sequence
  CAR component
agggtcaaattttctagatctgca

SEQ ID NO: 42 - DNA, 417 bp, Artificial Sequence
  CAR construct
gatgcgccggcctatcaacaaggccagaaccagctctataacgtaagtcagcctcgccgtagaccctccggaaaggaagggcagcctcctcctcggacctagggaacgcgcccgccagagtcttgggtttaggtttggtccctagctatcacctctgcgacccggctgcaacttgtttaacctcagttttctaacccacaaaacaggtgtagtaattgtacctgtctcaaagggcgccataaagattacatgctttaatgtcttgatgcatggaacgagcttagtctagtgcctgccactagtgagcccccaagccatcagcaatttgcaaaggctagaggggggtggagaaggaatgtattagaatcttcccagaggaggggaaagcgatttcacactccctgtgcaatgggactg

SEQ ID NO: 43 - DNA, 399 bp, Artificial Sequence
  CAR construct
caaggccagaaccagctctataacgtaagtcagcctcgccgtagaccctccggaaaggaagggcagcctcctcctcggacctagggaacgcgcccgccagagtcttgggtttaggtttggtccctagctatcacctctgcgacccggctgcaacttgtttaacctcagttttctaacccacaaaacaggtgtagtaattgtacctgtctcaaagggcgccataaagattacatgctttaatgtcttgatgcatggaacgagcttagtctagtgcctgccactagtgagcccccaagccatcagcaatttgcaaaggctagaggggggtggagaaggaatgtattagaatcttcccagaggaggggaaagcgatttcacactccctgtgcaatgggactg

SEQ ID NO: 44 - DNA, 339 bp, Homo sapiens
agggtcaaattttctagatctgcagatgcgccggcctatcaacaaggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 45 - DNA, 252 bp, Artificial Sequence
  CAR construct
taagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgc

SEQ ID NO: 46 - DNA, 720 bp, Artificial Sequence
  enhanced GFP
atggtatccaagggtgaagaattgttcactggcgtagttcctatcttggtggagctcgacggagatgtcaatggacacaagttttcagtcagtggcgaaggtgagggtgatgcaacttacgggaagcttacactgaaattcatttgtactactggcaagttgccggtgccatggcccacgctggtaacgacccttacctacggtgtccaatgtttttctcgataccccgatcatatgaaacagcatgacttttttaaatctgctatgcccgagggatacgtacaggaaaggactatattttttaaggacgatggtaactataagacccgcgccgaagtcaagtttgagggagatactcttgttaatcgaatcgagctcaaggggatagattttaaggaggatggcaacattctcggccacaagctggagtacaattacaatagtcacaatgtttacataatggctgataagcaaaaaaacggcatcaaagttaattttaaaattaggcataatatagaagacggttcagttcaactcgccgaccactaccagcagaatacccctattggcgatgggccggttctcctgcctgacaaccattacctctcaacacagtctgctttgagtaaagaccctaacgagaagcgagatcatatggtcttgctcgagttcgtaacggccgccggtataaccctgggcatggatgagctctacaaataa

SEQ ID NO: 47 - DNA, 400 bp, Artificial Sequence
  CAR construct
cctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcacc

SEQ ID NO: 48 - DNA, 399 bp, Artificial Sequence
  CAR construct
attttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatg

SEQ ID NO: 49 - DNA, 399 bp, Artificial Sequence
  CAR construct
accgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacag

SEQ ID NO: 50 - DNA, 648 bp, Artificial Sequence
  Promoter sequence
gtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccacc

SEQ ID NO: 51 - DNA, 400 bp, Artificial Sequence
  CAR construct
gattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttct

SEQ ID NO: 52 - RNA, 20 nt, Artificial Sequence
  guide RNA
gauggaauccucuucaucua

SEQ ID NO: 53 - RNA, 20 nt, Artificial Sequence
  guide RNA
gacgcccccgcguaccagca

SEQ ID NO: 54 - RNA, 20 nt, Artificial Sequence
  guide RNA
agagucucucagcugguaca

SEQ ID NO: 55 - RNA, 20 nt, Artificial Sequence
  guide RNA
gggaaucaaaaucggugaau

SEQ ID NO: 56 - RNA, 20 nt, Artificial Sequence
  guide RNA
ggggccacuagggacaggau

SEQ ID NO: 57 - RNA, 20 nt, Artificial Sequence
  guide RNA
ugguacccaccuucacucuc

SEQ ID NO: 58 - RNA, 20 nt, Artificial Sequence
  guide RNA
ugccuuguuccugagaguga

SEQ ID NO: 59 - protein, 434 aa, Artificial Sequence
  CAR construct
AQSFGLLDPKLCYLLDGILFIYRVILTALFLRATNFSLLKQAGDVEENPGPMALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVK

SEQ ID NO: 60 - DNA, 1999 bp, Artificial Sequence
  CAR construct
ggcaggagctctgtgcacagagaacagcctcatctgctcgccttgtttccacctcccctcccattgccccaggttctttggccccacagcggccacatctgccgttggtgccaataggttttccaggagctggttgaggtgggagggagggagagggttgtgatcaggctgaggcatggggattggatatagtctccgtgtcatgatttatttggtcagtcagtcctagtgccaccctggggtaatggggatgtgttctcgtcaccttgggcctggctgaccagctttatctcttggcacagaggcacagagctttggcctgctggatcccaaactctgctacctgctggatggaatcctcttcatctatcgtgtcattctcactgccttgttcctgagagccaccaacttctctcttttgaagcaggccggagatgtggaagaaaatcctgggcccatggctcttcctgtgactgcccttctgctgcccctggctctcctgctccatgctgcccggccagagcaaaaacttatctctgaagaggacctcgacatccagatgacacagacaaccagcagcctcagcgccagcctgggggacagagtgaccattagctgccgggcctctcaggacatcagcaaatacctgaactggtaccagcagaaaccagatggcactgtcaagctgctgatttaccacacctccaggctccacagcggcgtgcccagtcgcttcagcggcagtgggagcgggacagattattccctcacaatctccaacctggagcaggaagatattgccacatacttctgccagcaaggcaacaccctgccatacacatttggaggcggcaccaaattggagatcaccggcggtggtggatctggaggagggggcagcggaggtggcggctctgaggtgaaactgcaggagagtggccctggcctggtggctcccagccagagcctttctgtcacctgcaccgtgtctggggtgtccctgcctgactatggagtctcctggatccggcagcctccaagaaaaggactggaatggctgggcgtcatctggggaagtgagaccacctactataattcagccctcaagtcccggctcaccatcattaaggacaactccaaatcccaggtgttcctgaagatgaattctctccagactgatgacacagccatctactactgtgccaagcattattattacggcgggtcctatgccatggactactggggccaggggaccagtgtcactgtttcttctatagaagtaatgtatccccctccctacttggacaacgagaaatctaacggcacaatcatacacgttaagggcaagcatctgtgtccctcccctcttttccccggaccgtctaagccattttgggtcctcgtggtggtcgggggtgtgctggcctgctacagcttgctggtcacagtggccttcatcatcttctgggtgcgctccaagaggagccggctgcttcacagtgattacatgaacatgacccccaggaggccaggacccaccaggaagcactaccagccctacgctcccccgcgggactttgctgcttaccgcagcagagtgaaggtgggtaccactgggctttgggaggagggcacggggtcccccacttgatggatgttcagaggggccttggtcttggaaggtctcaagctcgggtggtgcctggggcttggtatccaggagcaaagcaaggaccagccaagtgtgtgccttgagtgggctgaggaggaggtggcagtgtctggctgagatggacagggtaggagggagagcctggtgctaggcacctccatgacaagccgtacaaatgtgtgcacatcagagtgtcccagggaaggcgatgctactggtgacaaaggggcttacactcaggcagaggtccttctttccaagtgtgaatgaaggccatgttagcctttctcttgaaaaggccctttcctcatctgtaactgggga

SEQ ID NO: 61 - protein, 492 aa, Artificial Sequence
  CAR construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 3175 bp, Artificial Sequence
  CAR construct
accgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagcattgattattgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggctcttcctgtgactgcccttctgctgcccctggctctcctgctccatgctgcccggccagagcaaaaacttatctctgaagaggacctcgacatccagatgacacagacaaccagcagcctcagcgccagcctgggggacagagtgaccattagctgccgggcctctcaggacatcagcaaatacctgaactggtaccagcagaaaccagatggcactgtcaagctgctgatttaccacacctccaggctccacagcggcgtgcccagtcgcttcagcggcagtgggagcgggacagattattccctcacaatctccaacctggagcaggaagatattgccacatacttctgccagcaaggcaacaccctgccatacacatttggaggcggcaccaaattggagatcaccggcggtggtggatctggaggagggggcagcggaggtggcggctctgaggtgaaactgcaggagagtggccctggcctggtggctcccagccagagcctttctgtcacctgcaccgtgtctggggtgtccctgcctgactatggagtctcctggatccggcagcctccaagaaaaggactggaatggctgggcgtcatctggggaagtgagaccacctactataattcagccctcaagtcccggctcaccatcattaaggacaactccaaatcccaggtgttcctgaagatgaattctctccagactgatgacacagccatctactactgtgccaagcattattattacggcgggtcctatgccatggactactggggccaggggaccagtgtcactgtttcttctatagaagtaatgtatccccctccctacttggacaacgagaaatctaacggcacaatcatacacgttaagggcaagcatctgtgtccctcccctcttttccccggaccgtctaagccattttgggtcctcgtggtggtcgggggtgtgctggcctgctacagcttgctggtcacagtggccttcatcatcttctgggtgcgctccaagaggagccggctgcttcacagtgattacatgaacatgacccccaggaggccaggacccaccaggaagcactaccagccctacgctcccccgcgggactttgctgcttaccgcagcagggtcaaattttctagatctgcagatgcgccggcctatcaacaaggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaagatctcgagtctagctcgagggcgcgccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttct

SEQ ID NO: 63 - protein, 500 aa, Artificial Sequence
  CAR construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSPGKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - DNA, 3199 bp, Artificial Sequence
  CAR construct
accgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagcattgattattgagtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggctcttcctgtgactgcccttctgctgcccctggctctcctgctccatgctgcccggccagagcaaaaacttatctctgaagaggacctcgacatccagatgacacagacaaccagcagcctcagcgccagcctgggggacagagtgaccattagctgccgggcctctcaggacatcagcaaatacctgaactggtaccagcagaaaccagatggcactgtcaagctgctgatttaccacacctccaggctccacagcggcgtgcccagtcgcttcagcggcagtgggagcgggacagattattccctcacaatctccaacctggagcaggaagatattgccacatacttctgccagcaaggcaacaccctgccatacacatttggaggcggcaccaaattggagatcaccggcggtggtggatctggaggagggggcagcggaggtggcggctctgaggtgaaactgcaggagagtggccctggcctggtggctcccagccagagcctttctgtcacctgcaccgtgtctggggtgtccctgcctgactatggagtctcctggatccggcagcctccaagaaaaggactggaatggctgggcgtcatctggggaagtgagaccacctactataattcagccctcaagtcccggctcaccatcattaaggacaactccaaatcccaggtgttcctgaagatgaattctctccagactgatgacacagccatctactactgtgccaagcattattattacggcgggtcctatgccatggactactggggccaggggaccagtgtcactgtttcttctcccgggaagccgaccacaacaccagcaccgcgcccaccaacaccagctcccacaatagcctcgcagccgctctcactgcgcccggaggcttgtaggcccgctgccggtggagcagttcatacgcgaggacttgatttcgcctgtgacatatatatttgggcacctctcgccggaacctgtggggtcttgctgctgtctctcgttataacgctttattgcaaacggggcagaaagaaactcctgtatatattcaaacaaccatttatgagaccagtacaaactactcaagaggaagatggctgtagctgccgatttccagaagaagaagaaggaggatgtgaactgagggtcaaattttctagatctgcagatgcgccggcctatcaacaaggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaagatctcgagtctagctcgagggcgcgccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttct

SEQ ID NO: 65 - RNA, 20 nt, Artificial Sequence
  guide RNA
ggccacggagcgagacaucu

(19) United States
(12) Patent Application Publication
(10) Pub. No.: US 2023/0406903 A1
(43) Pub. Date: Dec. 21, 2023

(54) REPROGRAMMING IMMUNE CELLS BY TARGETED INTEGRATION OF ZETA-DEFICIENT CHIMERIC ANTIGEN RECEPTOR TRANSGENES

(71) Applicant: Charité - Universitätsmedizin Berlin, Berlin (DE)

(72) Inventors: Dimitrios Laurin Wagner, Berlin (DE); Jonas Christian Kath, Berlin (DE); Michael Schmück-Henneresse, Berlin (DE); Hans-Dieter Volk, Berlin (DE); Petra Reinke, Berlin (DE)

(21) Appl. No.: 18/268,906

(22) PCT Filed: Dec. 22, 2021
(86) PCT No.: PCT/EP2021/087306

§ 371 Date: Jun. 21, 2023

(30) Foreign Application Priority Data
Dec. 22, 2020 (EP) 20216649.2

(51) Int. Cl. C07K 14/725; A61P 37/02; C12N 5/0783

(52) U.S. Cl. CPC C07K 14/7051; A61P 37/02; C12N 5/0636; C07K 2319/03; C12N 2510/00

(57) ABSTRACT

A nucleic acid construct for targeting and integrating a CD3 zeta-deficient chimeric antigen receptor (CAR) fragment into an endogenous CD3 zeta/CD247 gene of a host genome. Also disclosed is a genetically modified human cell expressing an exogenous nucleic acid sequence encoding a CD3 zeta deficient CAR fragment, integrated in-frame into the endogenous CD3 zeta/CD247 gene for gene fusion, to form a functional CAR including an exogenous CAR fragment fused with an endogenous CD3 zeta domain.

The invention relates to the field of genetic modification of therapeutic cell products. The invention relates to a nucleic acid construct for targeting and integrating a CD3 zeta-deficient chimeric antigen receptor (CAR) fragment (transgene) into an endogenous CD3 zeta/CD247 gene for generating a gene fusion and therefore a functional CAR comprising an exogenous CAR fragment fused with an endogenous CD3 zeta domain.

The invention further relates to said construct, comprising three nucleic acid sequence regions, wherein a first nucleic acid sequence region encodes a chimeric antigen receptor (CAR) fragment without a sequence encoding a functional intracellular domain of a CD3 zeta protein, a second nucleic acid sequence region comprises a targeting sequence configured for integrating the construct at an endogenous CD3 zeta gene of a human cell, and a third nucleic acid sequence region encoding and or comprising a protein separation site upstream of the first sequence region.

In further aspects, the invention relates to a CAR polypeptide as an in-frame fusion protein comprising the exogenous CAR fragment and an endogenous CD3 zeta intracellular domain with one or more immunoreceptor tyrosine-based activation motifs (ITAMs). The invention further relates to a genetically modified human cell expressing the CAR fragment, without a sequence encoding a functional intracellular domain of a CD3 zeta protein, integrated in-frame into the endogenous CD3 zeta/CD247 gene, to produce a fusion CAR comprising an exogenous CAR fragment with an endogenous CD3 zeta chain.

The invention further relates to a pharmaceutical composition comprising a genetically modified cell and a pharmaceutically acceptable carrier for therapeutic inventions in human diseases. The invention further relates to corresponding medical uses and therapeutic methods in the treatment and/or prevention of a medical condition, such as conditions associated with the presence of pathogenic cells expressing oncogenes, or for use in the treatment of an autoimmune disease or graft versus host disease.

### BACKGROUND OF THE INVENTION

The most promising immunotherapy strategy against cancer to date is the adoptive cell transfer (ACT) of genetically modified T lymphocytes. ACT has the potential to overcome many of the pitfalls associated with vaccine-based approaches to cancer treatment, such as the de novo activation and expansion of tumor-specific T cells in vivo, even in immunocompromised patients. Using cells genetically modified to recognize tumor-specific or tumor-associated antigens, ACT enables eradication of tumor and tumor stem cells and offers a robust form of immunotherapy to treat established tumors. In contrast to conventional chemo-, radiation- and surgical therapies, this approach potentially prevents tumor recurrence.

A major obstacle in the field of cancer immunotherapy is the breaking of tolerance to “own” antigens. Depending on the type of target, tumor-specific immune responses are directed against either “own” or “foreign” antigens. Foreign antigens can be produced in virally induced cancers or by genetic aberrations unique to a particular tumor, such as point mutations or chromosomal rearrangements that produce epitopes recognized by T cells and B cells. However, immune responses against tumor-associated self-antigens are subject to suppression by central or peripheral tolerance mechanisms. A major problem is the elimination of high-affinity T-cell receptors (TCRs) by the thymus, resulting in TCRs with relatively low affinity for self-antigens compared to foreign antigens being predominant.

There are a number of approaches to overcome the problem of tolerance in the application of cancer immunotherapy. One strategy is the genetic redirection and reprogramming of T cells with chimeric antigen receptors (CARs). Genes encoding antigen receptors can be introduced into the patient's T cells, which are then rapidly expanded to derive memory and effector lymphocytes capable of robust in vivo proliferation and strong anti-tumor activity. These synthetic receptors combine the effector functions of T lymphocytes and the ability of antibodies to recognize predefined surface antigens with high specificity in a non-MHC restricted manner.

The clinically most prominent example of this technology to date is the CAR-T cell therapy for CD19-positive tumors. Here, the patient's T-cells are expanded outside the body in a clean room laboratory and equipped with a CD19-recognising CAR construct by means of retro/lentiviral gene transfer. After infusion into the patient, the genetically modified CAR-T cells recognize the CD19 antigen on B cells and on cancer cells that arise from the B cell line. The clinical success in the treatment of corresponding lymphomas and leukemia has led to the clinical approval of two commercial CAR-T cell products in the USA and Europe (Tisagenlecleucel (Kymriah), Novartis and Axicabtagen-Ciloleucel (Yescarta), Kite/Gilead), others are in the pipeline.

Known CARs usually consist of an extracellular antigen-binding domain, which is connected to signal molecules inside the cell via a linker and a transmembrane domain. CAR constructs are variable in their composition. A “first generation” CAR has a signaling chain for primary T cell activation, usually the CD3 zeta signaling chain; a “second generation” CAR uses the CD3 zeta chain together with a costimulatory unit, preferably CD28 or 4-1 BB, to provide a sustained T cell to achieve activation. The “third generation” CAR includes multiple co-stimulatory signaling modules, such as CD28 and 4-1 BB (or others), in addition to CD3 zeta.

After interaction with the target antigen, receptor clustering at the membrane and conformational changes occur, which promote growth and survival via co-stimulatory domains and mimic a T-cell receptor signal via the CD3-zeta chain. The latter activates the cytotoxic function of T cells and also stimulates the production and release of cytokines. All functional components of CAR aside from the antigen-binding domain are naturally part of the genome of human cells.

A major disadvantage of the prior art technology are complications in the clinical implementation of CAR-T cell therapy (Elsallab et al. 2020), such as:
a) the complexity of producing an autologous cell product, especially in intensively pre-treated cancer patients,
b) the enormously high price (Kymriah, Novartis=320.000€) of therapy with an autologous cell product,
c) the critical to fatal side effects for the patient when applying a universal, hence allogenic, CAR-T cell product,
d) the exhaustion of the transgenic T cell product due to a constitutively expressing promoter, and e) the inefficient gene transfer and integration due to the size of the construct inserted, in particular when gene transfer is performed via non-viral and site-specific gene insertion.

Allogeneic T-cell products have a high risk of leading to the often fatal acute graft-versus-host (GvHD) reaction, in which the donor's CAR-modified T-cells triggered by their endogenous TCR recognize and attack the patient's tissue as “foreign”. The use of highly efficient and specific nucleases, such as TALENs and CRISPR-Cas9, has made it possible to specifically switch off genes for the TCR of the donor's CAR-modified T cells (Gundry et al. 2016, Osborn et al. 2016). An initial study with an allogenic TCR-deleted CAR-T cell product showed promising results. However, despite TCR-deletion, one of the patients developed mild GvHD and T-cells from the donorwere detected in the skin (Quasim et al. 2017). In the study, the CAR transgene was randomly integrated into the genome of the T-cells by means of a retrovirus and the TCR deletion was subsequently achieved in a separate step. The disadvantage of this technology is that, despite subsequent sorting of the cells, still about 0.1% of all CAR-T cells in the final cell product continue to express their original TCR. This may explain the development of GvHD.

Another established strategy allows the combination of CAR insertion and TCR deletion. In this case the CAR insertion automatically leads to TCR deletion. For this purpose the CAR transgene is inserted into the human TRAC gene locus, via a TRAC knock-in strategy, which encodes a part of the TCR-alpha chain (Eyquem et al. 2017). This leads to a TRAC knock-out and a loss of surface expression of the TCR-CD3 protein complex. In this study, homology-mediated gene transfer specific for exon 1 of the TRAC gene was initiated using CRISPR-Cas9 and an adenovirus-associated virus (serotype 6) to provide the DNA repair template. The CAR transgene including a poly-A terminator sequence is flanked by TRAC gene homologous sequences. These homology regions mimic the sister chromosome to the respective DNA section, which carries the double-strand break induced by the inserted nuclease. Thus, the DNA repair machinery of the T cells inserts the CAR transgene into the TRAC gene locus. This provides a TRAC gene locus controlled expression of CAR in T cells in the absence of a TCR complex. The TRAC modified CAR-T cells showed an improved tumor eradication potential (Eyquem et al. 2017, MacLeod et al. 2017). A weakness of the strategy is that a subset of T cells is able to use alternative TCRs independent of the TRAC gene, such as γδ-T cells. No TCR knock-out takes place in these cells, which makes allogenic use more difficult and the risk of GvHD still remains. This prior art technology is also not suitable for immune cells with potent anti-tumor function, such as natural killer cells, which do not express the TRAC gene and therefore cannot be reprogrammed using a knock-in at the TRAC locus.

To the knowledge of the inventors, all universal CAR-T cell products described to date aim for a complete TCR-KO and employ a complete CAR transgene including all its intracellular domains, in particular including the CD3-zeta domain, to redirect their cytotoxic activity. Further disadvantages in the prior art are the use of viral vectors and the size of the gene constructs to be transferred, which on the one hand are complex and cost-intensive in production under GMP conditions (Qasim et al. 2017 and Eyquem et al. 2017). On the other hand, non-viral methods for site-specific gene insertion are associated with low gene transfer efficiency due to the size of the transgenes (Roth et al. 2018, Nguyen et al. 2020).

Therefore, improving CAR immunotherapy requires overcoming complex technical and biological problems. The present invention addresses four challenging areas in CAR nucleic acid construct design, such as improving integration and expression, reducing the risk of side effects, the development of a TCR complex-deficient CAR-expressing therapeutic cell, including a CAR-expressing therapeutic NK cell, and improving therapeutic immune cell effectiveness.

### SUMMARY OF THE INVENTION

The invention addresses the above-mentioned challenges by (1) enabling CAR transgene expression under the intrinsic control of a TCR complex component promoter and the endogenous 3′-UTR (without the need for a transgenic transcription termination sequence) by taking advantage of intracellular gene regulatory elements (2) providing TCR complex-deficient therapeutic cells, (3) ensuring cell persistence of CAR-expressing therapeutic cells by taking advantage of intrinsic control of CAR transgene expression in immune cells, and (4) enabling efficient manufacturing and provision of “off the shelf” therapeutic immune cells.

In light of the prior art, the technical problem underlying the present invention is to provide a novel strategy for immunotherapy using genetically engineered therapeutic immune cells. In particular, a problem underlying the invention is the provision of means for an intrinsic, immune cell-specific and target-dependent transgene expression in therapeutic immune cells, while avoiding exhaustion, activity loss, and differentiation of said therapeutic immune cells. More specifically, one problem underlying the invention is the provision of means for allowing CAR fragments to be integrated into the CD247 gene in immune cells that are not T cells, such as NK cells, and expressed under regulation of the endogenous CD247 promoter, while presenting a functional CAR fragment on the surface of such cells.

A further problem underlying the invention is the provision of TCR complex-deficient therapeutic immune cells, such as allogeneic T cells, for immunotherapy of cancer while avoiding adverse effects caused by allogeneic immunotherapy.

Another problem underlying the invention is the provision of means for an efficient transfer and genomic integration of a transgene while avoiding the complex and cost-intensive production of viral vectors under GMP conditions. A further problem to be solved by the underlying invention is the provision of a fast-track and optimized immunotherapy, at lower cost.

These problems are solved by the features of the independent claims. Preferred embodiments of the present invention are provided in the dependent claims.

The invention therefore relates to a nucleic acid construct, comprising:
(a) a first nucleic acid sequence region, encoding a chimeric antigen receptor (CAR) fragment without a sequence encoding a functional intracellular domain of a CD3 zeta protein,
(b) a second nucleic acid sequence region, comprising a targeting sequence configured for integrating the construct at an endogenous CD3 zeta gene of a human cell, and
(c) a third nucleic acid sequence region encoding and or comprising a protein separation site upstream of the first sequence region.

In one embodiment, the nucleic acid construct is configured for integrating the construct (in particular the first and the third nucleic acid sequence regions) at a single site, preferably into a CD3 zeta/CD247 gene, in the genome of a human cell, preferably a human immune cell.

In one embodiment, the first nucleic acid sequence directly follows, i.e. is adjacent and down-stream with respect to the reading frame, the third nucleic acid sequence encoding or comprising a protein separation site.

In one embodiment, the first nucleic acid region encoding a CAR fragment is integrated into the genome of an immune cell, wherein integration of said first region disrupts or reduces endogenous expression of a protein selected from the group consisting of CD3 gamma chain, CD3 delta chain, CD3 epsilon chain, and CD3 zeta chain.

In one embodiment, the first nucleic acid sequence encoding a CAR fragment, is suitable for integration into a CD3 zeta/CD247 gene of the immune cell, preferably downstream of the promotor of said gene, such that expression of said first nucleic acid sequence is under control of an endogenous promoter and preferably a poly-A signal by taking advantage of intracellular gene regulatory elements of said gene within the immune cell. In some embodiments, the integration site is an exon of the endogenous gene expressed by the endogenous promoter in the immune cell.

In one embodiment, the first region encodes a CAR-fragment that is nonfunctional or has substantially reduced function. For example, the CAR fragment lacks a functional intracellular CD3 zeta domain of a functional CAR. Typically, a functional CAR comprises a TCR complex component with at least one ITAM domain, wherein a protein of the CD3 chain of the TCR complex includes at least one ITAM domain. A functional CAR of the prior art typically comprises an intracellular CD3 zeta chain, which is absent in the construct of the invention. The construct of the invention is designed and/or configured for targeting the endogenous CD3 zeta/CD247 gene and subsequent expression of a functional CAR from the combination of the exogenous CAR fragment in frame with the endogenous CD3 zeta coding region, under regulatory control of (expression by) the endogenous CD3 zeta promoter, and other intronic or 3′-UTR located regulatory elements and followed by the endogenous poly-adenylation signal.

In one embodiment, the present invention provides a nucleic acid construct that leads to prevention of formation of the TCR-CD3 complex at the surface of an engineered human CAR-T cell product.

In one embodiment, the invention relates to a nucleic acid construct comprising one or more targeting sequences configured for integrating said first sequence region (or the construct itself) at an endogenous CD3 zeta/CD247 gene of a human cell. In a preferred embodiment, an incomplete and non-functional CAR transgene is inserted into the endogenous CD3 zeta/CD247 gene of a human cell by homology-mediated DNA repair, whereby the CAR fragment transgene to be inserted contains neither CD3 zeta nor expression regulatory components, e.g. promoter or poly-A Signal, and is therefore shorter than the CARs to be transferred in the state of the art (refer to Example 1).

A skilled person would not have expected that integration of an incomplete, non-functional CAR fragment transgene into the CD3 zeta/CD247 gene would lead to reliable formation of a functional CAR transcript comprising the exogenous CAR transgene complemented with the endogenous CD3 zeta chain, expressed at levels sufficient for therapeutic application. In addition, the insertion of the CD3 zeta-deficient CAR transgene into an exon of the CD3 zeta/CD247 gene, which encodes the intracellular protein, also surprisingly produced a TCR-negative CAR-T cell (as shown in Example 2). An advantageous and unforeseeable aspect of the invention is the provision of a functional, correctly spliced CAR comprising the exogenous CAR transgene complemented with the endogenous CD3 zeta chain, which is presented on the surface of the human immune cell, e.g. a CAR-T cell or CAR-NK cell, that enables therapeutic function. The CAR is also efficiently expressed at the surface of non-T-cells for producing the therapeutic effect, such as leading to tumor eradication. Cell persistence of said cells is enhanced. Furthermore, the side effects, such as rapid cell exhaustion and cytokine storm, are minimized or completely avoided.

In one embodiment, the first nucleic acid sequence region encoding a CAR fragment is configured to be integrated into the genome of a cell such that the CAR is expressed by the immune cell at the surface of said cell, and wherein integration of said first nucleic acid into the genome disrupts or reduces expression of a functional TCR complex, if any, at the surface of the cell.

In one embodiment, the first nucleic acid sequence region encoding a CAR fragment is configured to be integrated into a chromosomal sequence on at least one allele of an endogenous gene, preferably two alleles of said gene, preferably an endogenous CD3 zeta/CD247 gene, more preferably two alleles of the CD3 zeta/CD247 gene.

The CAR-T cell product of the present invention, generated using the construct described herein, is in some embodiments also a TCR-negative CAR-T cell product which is particularly well suited for use as an “off-the-shelf” CAR-T cell product in cancer therapy with allogeneic cell therapy products or for cellular immunosuppression approaches in regenerative approaches. The TCR absence on CAR-expressing T cells preferably reduces or negates the risk of graft versus host disease.

In the prior art, CAR fragments are preferably introduced into T cells and induced to be expressed in these cells, especially allogeneic T cells. This has the risk of side effects, such as a cytokine storm. The expression of CAR fragments incorporated into the CD247 gene in immune cells other than T cells, e.g., NK cells, is addressed in the prior art by an exogenous promoter, with the disadvantage of achieving an artificial expression rate of the CAR fragment incorporated into the CD247 gene, poor presentation of the CAR fragment on the cell surface, and rapid depletion of the CAR-expressing cells and low cell endurance, that may lead to relapse in patients. Expression of functional CAR fragments integrated into the CD247 gene and under regulation of the endogenous CD247 promoter, for example, has not been disclosed in the prior art.

In some embodiments, the invention therefore relates to a nucleic acid construct and genetically modified immune cells expressing the first and the third nuclei acid sequence regions of said construct, preferably a CAR-T cell product or CAR-NK cell product conferring human T cells or NK cells a high cytotoxic activity against defined antigens, such as those of solid or liquid tumors, while sparing non-pathogenic cells, for example inside the tissue surrounded by a tumor, such as breast, pancreatic, prostate, lung, colon or liver cells as well as hematological cells.

In preferred embodiments, essentially all other endogenous T cells, B cells, NK cells are likewise spared as the CAR-T cell or CAR-NK cell products of the invention show no or negligible activity against these cells.

The present invention enables the stimulation of cells involved in an anti-tumor immune response and thereby the local activation, support and/or strengthening of an anti-tumor immune response. The present invention enables an effective and therapeutically relevant dose of one or more chimeric antigen receptors endogenously regulated through integration of a nucleic acid construct encoding a CD3 zeta-deficient CAR fragment into an endogenous gene, preferably CD3 zeta coding gene, in transplanted CAR-T cells or CAR-NK cells, while avoiding/suppressing TCR expression. TCR expression in allogeneic CAR-T cells are at times associated with severe side effects, e.g. GvHD. The present invention provides a strategy and method that avoids adverse TCR expression, while disrupting CD3 zeta expression essential for a functional TCR complex formation. In a further aspect, the invention provides a strategy and a method for employing a CD3 zeta-deficient CAR transgene, for example, dependent on immune cell specificity, site, expression state (e.g. expression of a transgene only in the vicinity of a tumor) or timing (e.g. before or after intervention in a tumor cell), wherein CAR expression is preferably under the control of an endogenous promoter and an endogenous Poly-A signal, preferably a CD3 zeta/CD247 gene promoter. CAR expression strategies in the prior art typically provide CAR-T cells, wherein CAR expression is regulated under an exogenous constitutive active RNA-polymerase II promoter combined with other exogenous gene regulatory elements.

In a preferred embodiment, the CD3 zeta/CD247 locus is the target of construction integration, whereby a CAR fragment is transferred to the CD3 zeta/CD247 locus, preferably downstream of the CD3 zeta/CD247 promoter. In a further embodiment, the expression of the CAR transgene takes place under control of the endogenous CD3 zeta/CD247 promoter and poly-A signal, whereby the CAR transgene preferentially forms a fusion gene with the endogenous CD3 zeta/CD247. The preferential integration and fusion of the CAR transgene into the endogenous CD3 zeta/CD247 gene leads in particular to loss of the endogenous CD3 zeta/CD247 gene expression and formation of the natural CD3 zeta protein. In one embodiment, loss of CD3 zeta/CD247 expression also leads to the suppression or absence of expression of a functional TCR complex, resulting in TCR-negative CAR T cells.

In one embodiment, a fusion gene is formed by a CAR transgene preferably integrated in frame into the expression pattern of CD3 zeta/CD247 gene, whereby an open reading frame for a truncated CD3 zeta/CD247 protein is maintained downstream of the CAR transgene insertion site, preferably for a truncated CD3 zeta/CD247 protein with at least one intracellular ITAM domain. In one embodiment, a CAR-CD3 zeta fusion protein is expressed under control of the endogenous CD3 zeta promoter with simultaneous loss of endogenous CD3 zeta/CD247 expression (refer to Examples 2 and 3).

It was a surprising result that the natural immune response could indeed be mirrored or enhanced, whereby a functional CAR at the cell surface was induced by the integration of a CD3 zeta-deficient CAR fragment into the endogenous CD3 zeta/CD247 gene in CAR-T cell (refer to Examples 3 or 5) or CAR-NK cell (Example 4) products by the means of the invention.

The truncated CAR constructs, described herein, are efficiently expressed in primary and immortalized NK cells under regulation of the endogenous CD247 promoter and translated into the functional CAR-CD3 zeta fusion protein. In one embodiment, the CAR construct is expressed in NK cells intrinsic control of the endogenous CD247 promoter and presented functionally on the cell surface of said NK cells. From the prior art, it was not expected that endogenous expression of the CAR construct in immune cells other than T cells, particularly NK cells, is sufficient to recognize and target tumor cells and mediate tumor eradication. Surprisingly, the artificial CAR fragment is fully expressed with functional intron excision elements and protein cleavage sites leading to translation of an intact and functional fusion protein comprising the CAR fragment and CD3 zeta element.

The CAR-CD3 zeta fusion protein is particularly advantageously transported to the cell surface and anchored through the transmembrane domain within the membrane, wherein the CAR moiety comprising the antibody structure are functionally expressed at the cell surface also in immune cells that are not T cells, preferably NK cells. In one embodiment, the CAR-CD3 zeta fusion protein is transported to the cell membrane of immune cells, preferably NK cells. In one embodiment, the CAR-fragment of CAR-CD3 zeta fusion protein is expressed and functional at the cell surface of immune cells, preferably NK cells.

The functionality of the CAR, mediated by the presentation of the CAR at the cell surface, particularly NK cells, is characterized by an optimal binding of the CAR antibody domain to an antigen presented at the surface of a tumor cell. Said binding triggers a signaling transduction cascade via the CD3 zeta domain fused to the CAR fragment, mediating tumor cell destruction. In one embodiment, the CAR antibody domain of said CAR on the cell surface of the NK cell recognizes and binds to an antigen presented at the surface of a tumor cell, wherein said binding triggers a signaling transduction cascade for tumor cell destruction. Primary and immortal NK cells produced with the CD3 zeta/CD247 KI approach provide a surprising killing capacity that is preferably advantageous over prior art.

Particularly beneficial are the reduced cell exhaustion of CAR expressing cells, if any, and enhanced cell persistence of the CAR-presenting therapeutic cells, particularly NK cells. The enhanced cell persistence of said CAR-presenting NK cells is particularly beneficial for complete tumor eradication. A cytokine storm is not induced or minimized. Surprisingly, relapses of patients during or after treatment are minimized or completely avoided.

In one embodiment, in NK cells, the CD3 zeta deficient CAR transgene as provided by the invention can be expressed under control of the CD3 zeta/CD247 promoter but not under the TRAC promoter (refer to Example 4). The CD3 zeta protein is expressed in NK cells at sufficient levels (refer to Example 4 and 16) and is not essential for other NK cell functions, e.g., killing of tumor cells/missing-self recognition (refer to Example 4). The superior killing capacity of primary CD247 KI NK cells and immortalized CD247 KI NK cells are preferably advantageous over prior art CAR NK cells that overexpress the CAR from an ectopic locus and/or a safe harbor (e.g. hAAVS1) (refer to Example 16). Another advantageous aspect of the invention is therefore targeting the gene encoding the CD3 zeta chain for integrating the CAR fragment transgene of the invention, since the CD3 zeta/CD247 gene is also expressed in immune cells, in addition to T-cells, which are also applicable for effective and safe immunotherapy, including NK-cells (refer to Examples 4 and 12). The CD247 KI approach, as described herein, also allows reprogramming of different cell types for adoptive cell transfer. For example, TCR γ/δ T cells, Tregs and NK cell can be reprogrammed by CD247-KI approach (refer to Example 13 and 4). The invention provides means for genetically modifying the endogenous CD247 gene in various immune cells for expressing an antigen specific CAR, wherein said immune cells exhibit significantly less exhaustion after tumor encounter in vitro. In one embodiment, TCR γ/δ T cells can be genetically modified using the CD247 KI approach and used for adoptive T cell transfer for cancer therapy. The TCR complex component TRAC, as used in the prior art as integration site (WO2017/1809893), does not provide this advantage.

The combination of the CD3 zeta deficient CAR transgene integrated into the CD3 zeta/CD247 gene locus, with NK cells or other immune cells, such as Treg, is a preferred embodiment of the present invention.

It was surprising and is particularly advantageous, that by integrating the CAR transgene into the CD3 zeta/CD247 gene, in particular the region of the gene encoding for the cytoplasmic domain, the endogenous CD3 zeta expression is disrupted and no endogenous TCR is presented on the CAR T cell. It was not expected that the construct of the invention would directly influence expression of the intracellular human CD3 zeta chain. The present invention therefore also provides means to disrupt the endogenous TCR expression and to enable a safe immunotherapy with allogeneic CAR-T cells, thus making immunotherapy not only safer but also easier, faster and significantly cheaper. Furthermore, it was surprising that the targeted immune cells correctly splice the CAR transgene integrated in the CD3 zeta/CD247 gene without exonic sequences of the CD3 zeta chain, in order to form a functional CAR transcript complemented with an endogenous CD3 zeta domain.

In one embodiment, the insertion of a truncated CD3 zeta-deficient CAR transgene into the endogenous CD3-zeta/CD247 gene allows for a sufficient expression of the CAR (refer to Example 7). It was surprising that the level of expression of CAR in the present invention, which is lower than that of prior art CARs, leads to a sufficient and therapeutically relevant expression of the CAR in CAR-T cells or CAR-NK cells (refer to Example 8). It was not expected that this lower expression rate of the CAR, as a CAR-CD3 zeta fusion protein in the CAR-T cell and CAR-NK cell, which corresponds to the endogenous expression rate of CD3 zeta/CD247 in said cells, is particularly advantageous for efficacy in tumor eradication and safety of immunotherapy (refer to Example 3). Of particular advantage is that the mentioned CAR-T cells or CAR-NK cells show a reduced cell depletion and differentiation associated with the improved anti-tumor activity and expansion of the CAR-T cell or CAR-NK cell products.

The endogenous regulation of expression was surprisingly beneficial for the performance and endurance of CAR-T cells and CAR-NK cells of the invention described herein.

The present invention therefore provides means for the production of CD3 zeta promoter-dependent expression of a CD3 zeta deficient CAR in CAR-T cells or CAR-NK cells with a potent and antigen-specific ability to eliminate tumor cells in vitro (refer to Examples 8 and 9). Integration of a CD3-zeta-deficient CAR into other genomic loci than the CD3 zeta/CD247 gene lacks a cytotoxic effect. As a result, fully functional, TCR-negative CAR-T cells can be produced by a precisely positioned insertion of the CD3 zeta-deficient CAR transgene.

In some embodiments, the provision of a combination of transgenes encoding a tumor-specific CAR, an immune stimulating protein, e.g. cytokine or checkpoint inhibitor molecule, a CAR-regulating protein and/or a therapeutic protein in CAR-T cells or CAR-NK cells, also achieves a locally more effective and safer anti-tumor response.

In some embodiments, additional sequences can be placed within the construct, preferably in the first nucleic acid sequence, wherein these sequences encode one or more therapeutic proteins that are separated from the CAR fragment-encoding region by a sequence encoding a self-cleavage peptide, proteolytic cleaving site, and/or an IRES site.

The combination with additional therapeutic proteins, preferably expressed under the same promoter as the CAR fragment, leads to a unique local expression and secretion of such proteins, which induces a local anti-tumor response encompassing multiple arms of the immune response without inducing toxicity as frequently observed in the application of immunostimulatory molecules, e.g. cytokines, in tumor patients.

A further advantage of the invention is the cell type-dependent regulation of gene expression for the inserted CAR transgene. The CD3 zeta/CD247 gene is expressed in cells of the immune system, preferably in lymphocytes, such as T cells and NK cells. Other cell types, e.g. muscle cells, do not express CD3 zeta/CD247 or at a very low extent. Thus, the strategy and methods of the invention also allow that CAR transgenes, which are misguidedly transferred into non-target cells and inserted into their genome, remain non-expressed in these non-target cells or show an expression rate without any therapeutic or toxic relevance.

In one embodiment, the first nucleic acid sequence region encoding the CAR fragment comprises:
a nucleic acid sequence encoding an extracellular antigen-binding domain, said antigen-binding domain preferably comprising an antibody or antibody fragment,
a nucleic acid sequence encoding a transmembrane domain, and
a nucleic acid sequence encoding one or more intracellular co-stimulatory domains.

In one embodiment, the CAR antigen-binding domain binds to a cancer antigen. In some embodiments, the antigen binding domain binds to a cancer associated antigen selected from the group consisting of: CD19, APRIL/TNFSF13, Mic A/B, T cell associated antigens: CD3, CD5, CD7, Folate receptor alpha (FRa), ERBB2 (Her2/neu), EphA2, IL-13Ra2, epidermal growth factor receptor (EGFR), Mesothelin, TSHR, CD123, CD22, CD30, CD171, CS-1, CLL-1, CD33, EGFRvIII, GD2, GD3, BCMA, Tn Ag, prostate specific membrane antigen (PSMA), ROR1, FLT3, FAP, TAG72, CD38, CD44v6, CEA, EPCAM, B7H3, KIT, interleukin-11 receptor a (IL-11 Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-I receptor, CAIX, LMP2, gp100, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p⁵³ mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, and derivates thereof comprising post-translational modifications of the polypeptide, for example, glycosylations, ubiquitination, acetylations, phosphorylations and the like, as well as other modifications known in the art, both naturally occurring and non-naturally occurring.

In one embodiment, the CAR antigen-binding domain binds to a self- or alloantigen, wherein the self- or alloantigen is selected from a group of HLA haplotypes including HLA-A2, HLA-B7, HLA-DR as well as HLA associated antigens, such as Mic A, or Mic B.

In one embodiment, the CAR antigen-binding domain binds to a self-antigen associated with inflammation or an exogenously introduced antigen, wherein said exogenously introduced self-antigen can be selected from a group including MOG, Aquaporin 4, Gluten-tissue transglutaminase complex, adenovirus, or AAV.

In some embodiments, an immune cell, preferably NK cell or T cell, in which the first nucleic acid encoding the CAR fragment is expressed, is sensitized to a target antigen. In one embodiment, the antigen binding domain of the CAR fragment recognizes (preferably specifically binds to) CD19. Corresponding immune cells expressing said CAR fragment, preferably a CAR-T cell or CAR-NK cell product, exhibit a high cytotoxic activity against CD19 positive pathogenic cells.

In one embodiment, the antigen binding domain of the CAR fragment recognizes (preferably specifically binds to) HER2. Corresponding immune cells expressing said CAR fragment, preferably a CAR-T cell or CAR-NK cell product, exhibit a high cytotoxic activity against HER2 positive pathogenic cells.

In another aspect, the invention also relates to a cell product, such as an advanced therapy medicinal product, designed and manufactured for medical use, comprising next generation anti-CD19 CAR-transfected or transduced immune cells that can be used in the treatment of cancer with CD19 positive tumor cells (refer to Example 8).

Besides targeting CD19, the Zeta approach is transferable to other CAR targets (e.g. HER2, refer to Example 15). In one embodiment, the antigen binding domain of the CAR fragment recognizes (preferably specifically binds to) HER2. Corresponding immune cells expressing said CAR fragment, preferably a CAR-T cell or CAR-NK cell product, exhibit a high cytotoxic activity against HER2 positive pathogenic cells. In another embodiment, the cell product comprising next generation anti-HER2 CAR-transfected or transduced immune cells can be used in the treatment of cancer with HER2 positive tumor cells. The anti-HER2 CAR expressing immune cells, such as T cells or NK cells, display reduced exhaustion marker expression after in vitro expansion. The differentiation of anti-HER2 CAR T-cells is beneficially reduced.

The present invention therefore provides a specific guide RNA sequence for targeting the CD3 zeta/CD247 gene that can be also applied for NK cells, for example for producing CAR-NK cells targeting HER2. The guide RNA disclose herein most efficiently leads to a specific integration of the nucleic acid construct comprising the CAR fragment into CD3 zeta/CD247 gene at the preferred position, such as a CAR fragment targeting CD19 or HER2. Such specific guiding for exact integration is critical for a precise synthesis of the CAR/CD3 zeta fusion protein and functionality of the CAR fragment, such as surface presentation and targeting, and is a advantageous feature of the guide RNA disclose herein over guide RNAs in prior art.

Further embodiments of the first nucleic acid sequence region encoding the CAR fragment are listed below. In some embodiments, the nucleic acid region encoding the antigen binding domain of the CAR described herein, encodes an antigen binding domain coupled to a transmembrane domain by a hinge region.

In some embodiments, the transmembrane domain comprises a protein selected from the group consisting of the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, and CD154.

In some embodiments, the intracellular signaling domain comprises a costimulatory signaling domain comprising a functional signaling domain obtained from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, gp130, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a or derivates thereof with at least 80% amino acid sequence identity.

In one embodiment, the nucleic acid construct encodes a CD3 zeta deficient CAR fragment, comprising:
a CAR signal sequence, preferably according to SEQ ID NO 27 or a sequence with at least 80% sequence identity to SEQ ID NO 27;
an antigen-binding domain of a CAR that specifically binds to an antigen, more preferably CD19 according to SEQ ID NO 28 and SEQ ID NO 30 linked with a linker according to SEQ ID NO 29, or CD19Y according to SEQ ID NO 60 with a linker according to SEQ ID NO 29, or a sequence with at least 80% sequence identity to SEQ ID NO 28, 29, 60, and 39;
an immunoglobulin heavy chain constant region of a CAR, preferably according to SEQ ID NO 31-33, or a sequence with at least 80% sequence identity to SEQ ID NO 31-33; and/or
a CD28 signalling domain, preferably according to SEQ ID NO 34 and 35 or a sequence with at least 80% sequence identity to SEQ ID NO 34 and 35; wherein the CD28 signaling domain comprises a transmembrane domain, preferably according to SEQ ID NO 34, or a sequence with at least 80% sequence identity to SEQ ID NO 34.

In some embodiments, the nucleic acid construct encodes a CD3 zeta deficient CAR fragment further comprising
a cytoplasmic CD3 zeta signaling domain comprising a stop signal for protein translation, preferably according to SEQ ID NO 44, or a sequence with at least 80% sequence identity to SEQ ID NO 44, and
a poly-A signal, preferably according to SEQ ID NO 45, or a sequence with at least 80% sequence identity to SEQ ID NO 45, or
a truncated CD3 zeta signaling domain lacking a stop signal for protein translation and lacking a poly-A signal, preferably according to SEQ ID NO 41, or a sequence with at least 80% sequence identity to SEQ ID NO 41.

In one embodiment, a poly-A signal is known to a skilled person and can be an endogenous poly-A signal within a gene structure, a synthetic transcription terminator sequence or a poly-A sequence adapted from mammalian genes.

In some embodiments, the sequence variants with 70% or more sequence identity to the specific CAR sequences of SEQ ID 28-33 maintain CD19 binding with essentially the same or similar functional properties as VH and VL domains with the specific CAR sequences of SEQ ID NO 28-33 or SEQ ID NO 60, i.e. the CD19 binding is essentially the same or similar with respect to affinity, specificity and epitope binding mode. In some embodiments, the nucleic acid sequence encoding a CD3 zeta deficient CAR fragment is codon-optimized in order to improve expression of said CAR fragment. Such modifications enable sufficient surface expression on T cells or NK cells and still maintain proper antigen binding. High affinity and high avidity enable CAR-T cells or CAR-NK cells or CAR-Treg cells to i) recognize, ii) be activated against, and iii) kill tumor target cells with high, intermediate or low tumor-antigen surface expression.

In one embodiment, the antigen-binding domain of the CAR fragment specifically binds CD19. The anti-CD19 CAR-T cell and the anti-CD19 CAR-NK cell product described herein are characterized by beneficial properties. The anti-CD19 CAR as described herein has a high affinity and confers high specificity and avidity to T cells and/or NK cells. These properties enable CAR-T cells or CAR-NK cells to i) recognize, ii) be activated against, and iii) kill tumor target cells with high or low CD19 surface expression. For example, the number of CD19 antigens expressed on the surface of tumor cells can be quantified using an anti-CD19 antibody coupled to a fluorescent-dye in conjunction with Quantibrite beads (e.g. from BD). The preferred method applied to quantify CD19 antigens expressed on the surfaces of tumor cells is “fluorescence activated cell sorting/cell analysis” (FACS).

Anti-CD19 CAR-T cells without TCR expression, in particular in allogenic CAR cell products, will enable targeting of the tumor cells in the tumor microenvironment as shown in the examples described herein (refer to Example 11).

In one embodiment, the antigen-binding domain of the CAR fragment specifically binds CD19. The anti-HER2 CAR-T cell and the anti-HER2 CAR-NK cell product described herein are characterized by beneficial properties. The anti-HER2 CAR as described herein has a high affinity and confers high specificity and avidity to T cells and/or NK cells. These properties enable CAR-T cells or CAR-NK cells to i) recognize, ii) be activated against, and iii) kill tumor target cells with high or low HER2 surface expression. For example, the number of HER2 antigens expressed on the surface of tumor cells can be quantified using an anti-HER2 antibody coupled to a fluorescent-dye in conjunction with Quantibrite beads (e.g. from BD). The preferred method applied to quantify HER2 antigens expressed on the surfaces of tumor cells is “fluorescence activated cell sorting/cell analysis” (FACS).

Anti-HER2 CAR cells without TCR expression, in particular in allogenic CAR cell products, will enable targeting of the tumor cells in the tumor microenvironment as shown in the examples described herein (refer to Example 15).

The tumor antigen-specific CAR-T cells described herein are applicable in preferred embodiments for the treatment of patients with solid or liquid tumors that are not eligible for other therapies. More specifically, the embodiments of the invention relate to the treatment of the following patient populations:
i. patients with multidrug resistances,
ii. patients not eligible for allogeneic/hematopoietic stem cell transplantation,
iii. patients with co-morbidities that preclude further chemotherapies,
iv. patients suffering from solid and/or liquid cancers,
v. aged patients who do not tolerate chemotherapies,
vi. the CAR is applicable for salvage therapies even after progressive disease and multiple lines of other standard of care therapies have failed, and/or
vii. it is applicable even at low antigen density on target tumor cells, where antibodies can fail, and/or
viii. it is applicable as a monotherapy which is not the case for antibodies, and/or
ix. it is applicable in combination with one or more anti-cancer treatments, anti-cancer medicaments or transplantation.

As demonstrated in the examples below, in an in vitro co-culture system, anti-CD19 CAR-T cells or anti-CD19 CAR-NK cells become activated upon exposure to CD19-expressing human tumor cell lines (refer to Example 8 and 9). These T cells or NK cells then develop an effector phenotype with high level of a cytotoxic activity. Additionally, a cytotoxicity assay against selected target cell lines, CD19-negative cells, shows that selective cellular cytotoxicity is obtained only against cell lines positive for CD19 (refer to Example 8).

In one embodiment, the targeting sequence is configured for integrating said first nucleic acid sequence region in-frame with the endogenous CD3 zeta encoding sequence, preferably into an exon of an endogenous CD3 zeta gene.

In one embodiment, the targeting sequence is configured for integrating the construct in-frame into an exon of an endogenous CD3 zeta encoding gene.

In one embodiment, an insertion site of the CD3 zeta deficient CAR transgene is preferably downstream of the CD3 zeta/CD247 promoter, wherein the insertion can take place into an exon or into an intron of the CD3 zeta/CD247 gene, preferably an exon. In some embodiments, the exon of the CD3 zeta/CD247 gene for insertion of said CAR transgene can be exon 1, exon 2, exon 3, exon 4, exon 5, exon 6, exon 7 or exon 8, preferably exon 1, 2, 3, 4, 5, or 6, more preferably exon 2 or exon 3, more preferably exon 2, wherein the resulting CAR polypeptide includes at least one intracellular ITAM domain.

In one embodiment, the CAR transgene encoded by said first nucleic acid sequence region is not operably linked to an exogenous promoter.

In one embodiment, the third nucleic acid sequence region positioned upstream of the first sequence region, encodes a first self-cleavage peptide, protease cleavage site or an internal ribosomal entry site (IRES).

In one embodiment, the protein separation site of the third nucleic acid sequence encodes an internal ribosomal entry site (IRES).

Sequences downstream of a promoter may be transcribed but are not necessarily translated. An untranslated region downstream of a promoter and extending to the first start codon for translation is the 5′ untranslated region (5′ UTR). In one embodiment of the invention, the insertion site for the CAR transgene is therefore downstream of the 5′ UTR.

In some cases, a sequence segment upstream of the CAR fragment is transcribed together with the exogenous CAR transgene. In some embodiments, means are therefore provided to separate any sequence upstream of CAR polypeptide during or after translation.

In some embodiments, a nucleic acid sequence region encoding a protein separation site, for example a self-cleavage peptide, a protease cleavage site or an internal ribosomal entry site (IRES), preferably a self-cleavage peptide, is positioned upstream of the nucleic acid sequence region encoding the CAR fragment. The protein separation site is employed to separate any sequences upstream of the CAR, that are translated together with the CAR, that may be dysfunctional or unwanted in the CAR polypeptide.

In one embodiment, the CAR fragment encoding region is therefore separated from endogenous gene and/or mRNA sequence segments by a sequence encoding a self-cleavage peptide, proteolytic cleavage site, and/or an IRES site.

In one embodiment, a polypeptide cleavage site is selected from the group consisting of P2A, T2A, E2A and F2A.

In one embodiment, the proteolytic cleavage site is an amino acid recognition site for an intracellular proteolytic cleavage enzyme.

In one embodiment, the IRES site, for example enabling mRNA translation initiation at the ribosome in a cap-independent manner, is a viral IRES site, e.g. HCV-like IRES, picornavirus IRES or encephalomyocarditis virus IRES.

In some embodiments, the self-cleavage peptide is preferably a 2A peptide, more preferably selected from the group consisting of a foot-and-mouth disease virus (FMDV) 2A peptide, an equine rhinitis A virus (ERAV) 2A peptide, a Thosea asigna virus (TaV) 2A peptide, a porcine tescho virus-1 (PTV-1) 2A peptide, a Theilovirus 2A peptide, and an encephalomyocarditis virus 2A peptide.

In one embodiment, the nucleic acid construct is configured for expression of a chimeric antigen receptor (CAR), comprising the CAR fragment encoded by the first sequence region and an intracellular domain encoded by the endogenous CD3 zeta gene, under control of an endogenous CD3 zeta promoter.

Such a construct enables integration of the transgene, encoding a CD3 zeta deficient CAR fragment, into a location within the genome, such that the integrated nucleic acid sequence, i.e. the transgene, is under the control of an endogenous promoter and an endogenous poly-A signal by taking advantage of intracellular gene regulatory elements. In some embodiments, the endogenous promoter is a promoter of a TCR complex component.

The endogenous promoter is therefore intrinsically regulated and/or induced, e.g. by cellular signals, from the immune cell. In some embodiments, the endogenous promoter is active in a subset of immune cells, preferably NK cells or T cells or Treg cells. In some embodiments, the endogenous promoter is preferably a promoter of genes encoding proteins of the TCR complex, e.g. a CD3 zeta, CD3 epsilon, CD3 delta, CD3 gamma, preferably a promoter of a gene encoding a protein comprising at least one intracellular ITAM domain.

In one embodiment, expression of the first nucleic acid sequence region after integration is terminated by an endogenous CD3 zeta gene expression termination signal and/or use of the endogenous poly-A signal.

It is a particular advantage of the present invention that it employs not only endogenous promoter expression, but also endogenous termination signals. The endogenous gene regulation of CD3 zeta/CD247 is a rate-limiting step in TCR complex formation and is influenced by tumor microenvironment. In addition, due to the absence of exogenous termination signals, the construct of the invention is smaller and results in a particularly effective transfer and integration of the nucleic acid sequences.

A surprising effect of the CD3 zeta-deficient CAR construct, lacking both an exogenous promoter and expression termination signals including a poly-A signals, is that it exhibits an unexpectedly low toxicity to target cells compared to prior art CAR constructs, which would not have been predicted by a skilled person. In preferred embodiments this is particularly evident for non-viral transfer methods, such as those disclosed herein. This is advantageous for target cell survival, transgene integration rate, and function of the targeted cell (refer to Examples 3 and 6).

In one embodiment, an endogenous CD3 zeta/CD247 gene expression termination signal comprises a transcription terminator sequence, a poly-A signal, and a translation stop codon, and is known to a skilled person, who is capable of identifying a suitable fusion site and/or generating nucleic acid constructs configured for fusion, depending on the intended target site for fusion within an endogenous CD3 zeta/CD247 gene.

In some embodiments, the target site of the CD3 zeta gene, into which the first sequence region is integrated, is an exon and/or an intron, preferably exon 2, and is positioned upstream of an endogenous CD3 zeta sequence encoding an intracellular domain or fragment thereof, said intracellular domain or fragment thereof comprising one or more immunoreceptor tyrosine-based activation motifs (ITAMs).

In some embodiments, the target site of the CD3 zeta gene, into which the first sequence region is integrated, is exon 2, and is positioned upstream of an endogenous CD3 zeta sequence encoding an intracellular domain or fragment thereof, said intracellular domain or fragment thereof comprising one or more ITAMs.

In some embodiments, the target site of the CD3 zeta gene, into which the first sequence region is integrated, is an exon, and is positioned upstream of an endogenous CD3 zeta sequence encoding an intracellular domain or fragment thereof, said intracellular domain or fragment thereof comprising one or more ITAMs.

In some embodiments, the target site of the CD3 zeta gene, into which the first sequence region is integrated, is an intron, and is positioned upstream of an endogenous CD3 zeta sequence encoding an intracellular domain or fragment thereof, said intracellular domain or fragment thereof comprising one or more ITAMs.

In some embodiments, an ITAM-comprising CD3 protein domain for a CAR can be CD3 zeta, CD3 delta, CD3 gamma, CD3 epsilon, preferably CD3 zeta. The intracellular parts of CD3 zeta, CD3 delta, CD3 gamma, CD3 epsilon each carry at least one conserved motif known as an activation motif based on immune receptor tyrosine, termed “ITAM”, which is essential for the TCR signaling, in particular to generate an activation signal in T lymphocytes. The endogenous intracellular domain of CD3 zeta contains three ITAM motifs, although not all are necessarily essential. In a preferred embodiment, the CAR fusion protein comprises a CAR fragment fused to functional CD3 zeta fragment with at least one ITAM domain.

The CD3 zeta protein comprises an extracellular and a transmembrane domain, followed by an intracellular domain comprising three ITAMs. For its function as a signaling domain within a CAR, at least one ITAM is essential. In certain embodiments, when selecting the integration site for the construct, a site within the CD3 zeta/CD247 gene is selected which is upstream of at least one ITAM, more preferably upstream of all three ITAMs. A skilled person is capable of identifying a sequence encoding an ITAM domain and a suitable site for an in-frame integration.

The coding sequence for the ITAM-containing intracellular domain of CD3 zeta begins in exon 2. In one aspect of the invention, exon 2 is therefore particularly advantageous as an insertion site, thereby producing a CAR without an endogenous extracellular or transmembrane region of CD3 zeta. In further embodiments, the insertion site for the CAR transgene can also be located in exon 3, exon 4, exon 5 or exon 6.

In some embodiments, the first nucleic acid sequence region encoding a CAR fragment additionally comprises a sequence encoding a CAR-regulating, immune-stimulating and/or therapeutic protein, in-frame and separated from the CAR fragment-encoding region by a sequence encoding a second self-cleavage peptide, a promoter and/or an IRES site.

The construct can therefore lead to expression of additional beneficial proteins, e.g. cytokine and/or checkpoint inhibitor molecules with the aim to attract immune effector and helper cells, to induce immune activation, promote maturation of memory immune cells and/or suppress the development and persistence of suppressive and/or regulatory immune cells.

In some embodiments, the first nucleic acid sequence region encoding a CAR fragment additionally comprises at least one sequence encoding an immune-stimulating protein, e.g. chemokines, cytokines and/or checkpoint inhibitors. Cytokine, chemokines, and checkpoint inhibitors that stimulate the immune response are known to a skilled person and can be assessed and/or determined by established routine assays. In some embodiments, the immune-stimulating protein that is a cytokine is selected from the group consisting of IL2, IL12, IL15, IL18, IL-7, or constitutively active cytokine receptors, exogenously activated co-stimulatory or cytokine signaling promoting receptors. This strategy avoids potential adverse effects of systemic application of said immune stimulatory proteins.

Tumor-induced immunosuppression is largely mediated by the secretion of anti-inflammatory cytokines by immune cells and control proteins, e.g. immune checkpoint proteins such as PD-1, present in a tumor that have a regulatory phenotype (e.g. regulatory T cells and suppressor cells derived from monocytes). The invention therefore additionally provides advantageous means for modifying the tumor microenvironment, making it pro-inflammatory, promoting the activation of immune cells present in the tumor and the recruitment and activation of external immune cells, thereby facilitating broad activation of the immune system against the tumor and/or increasing the effectiveness of anti-tumor immunotherapeutic treatments.

In one embodiment, the construct encodes a checkpoint inhibitory protein, e.g. a PD-L1 and/or PD-1 inhibitor. Cancer cells utilize mechanisms that avoid regular immune system control. Checkpoint proteins have been shown to function by communicating to the immune system that a potentially cancerous cell is not to be destroyed.

In one embodiment, a CAR-regulating protein is encoded. In some cases, it may be desirable to provide a safety mechanism that allows selective deletion of the administered T cells, as the manipulated T cells can potentially spread and persist for years after administration. The invention provides additional means to optionally switch off the genetically modified cell product expressing the CAR, as described herein, i.e. for safe clinical use. Therefore, in some embodiments the construct encodes a suicide gene, capable of reducing the risk of toxicity and/or uncontrolled proliferation of T cells after administration to a subject. Suicide genes enable the selective deletion of transformed cells in vivo.

In some embodiments, additional sequences within the first nucleic acid sequence region are inserted in-frame and separated from the CAR fragment-encoding region by a sequence encoding a self-cleavage peptide, proteolytic cleavage site, and/or an IRES site.

In one embodiment, the first sequence region encoding the CAR fragment is linked to additional nucleic acid sequence region encoding, for example, a CAR-regulating protein, an immunostimulatory protein or a checkpoint inhibitor molecule, and is configured to encode a polycistronic mRNA.

In some embodiments, the targeting sequence of the second sequence region is configured for integration into a host genome by a gene editing technique, preferably CRISPR-Cas, zinc finger nucleases (ZFNs), integrases, site specific recombinases, meganucleases, homing endonucleases, or TALENs, more preferably a CRISPR-Cas9 derived from Streptococcus pyogenes.

In one embodiment, a gene editing technique is used to create a double strand break, preferably at a target site in the CD3 zeta allele, and the nucleic acid construct is incorporated into the target site by homology directed repair at the site of the double-strand break. A non-viral vector is preferably used for transferring the nucleic acid construct encoding the CAR fragment into the cell. In one embodiment, a delivery method is non-viral, preferably electroporation (refer Example 6). The gene transfer of the CRISPR-Cas9 machinery with the construct can be carried out by electroporation or virus-mediated, e.g. lentivirus, or transposon-controlled.

In some embodiments, the nucleic acid construct, preferably a linear DNA, or a vector, such as a plasmid vector, viral vector, or a transposon vector, enables an unusually high transfer rate into human T cells or NK cells.

In a further aspect of the invention, the invention relates to an isolated nucleic acid molecule, preferably in the form of a vector, such as a synthetic linear DNA or a circular plasmid vector, viral vector or a transposon vector, preferably a plasmid vector, selected from the group consisting of:
i. a nucleic acid molecule comprising a nucleotide sequence,
   which encodes a CD3 zeta deficient CAR polypeptide according to any embodiment described herein,
   which encodes an extracellular antigen-binding domain, a transmembrane domain, and an intracellular co-stimulatory domain, or
   wherein the extracellular antigen-binding domain is encoded by at least one sequence of SEQ ID NO 28, 29, 30, 31, 32, 33, 60 and/or
ii. a nucleic acid molecule comprising a nucleotide sequence,
   which encodes a targeting sequence comprising identical sequences to a recognition site of the endogenous DNA sequence into which integration is intended, also termed homology arms, according to any embodiment of the CAR fragment described herein, wherein the homology arm is according to at least one sequence of SEQ ID NO 25, 36, 39, 42, 43,
   and/or
iii. a nucleic acid molecule comprising a nucleotide sequence,
   which encodes or comprises a protein separation site according to any embodiment described herein, wherein the protein separation site is encoded by at least one sequence of SEQ ID NO 26, and/or
iv. a nucleic acid molecule which is complementary to a nucleotide sequence in accordance with i, ii and/or iii,
v. a nucleic acid molecule comprising a nucleotide sequence having sufficient sequence identity to be functionally analogous/equivalent to a nucleotide sequence according to i-iv, comprising preferably a sequence identity to a nucleotide sequence according to i-iv of at least 70%;
vi. a nucleic acid molecule which, as a consequence of the genetic code, is degenerate to a nucleotide sequence according to i-v; and/or
vii. a nucleic acid molecule according to a nucleotide sequence of i-vi which is modified by deletions, additions, substitutions, translocations, inversions and/or insertions and is functionally analogous/equivalent to a nucleotide sequence according to i-vi.

In one embodiment, a gene editing technique may be used to generate a genetically modified immune cell that comprises a chromosomal sequence edited on at least one allele of an endogenous gene, preferably two alleles of said gene, preferably an endogenous CD3 zeta/CD247 gene, more preferably two alleles of the CD3 zeta/CD247 gene.

In another aspect, the invention further relates to a method for producing a genetically modified cell, comprising delivering or transferring a nucleic acid construct as described herein.

Additionally, the signaling components of the CAR encoding nucleic acid construct may be exchanged, for example in a three-step cloning procedure, that allows for a modular composition, and tailor-made construction by a skilled person, of clinically applicable anti-tumor-antigen CARs, for example anti-CD19 CARs (refer to Examples 10 and 11) or anti-HER2 CAR (refer to Example 15).

In one embodiment, the CD247 KI approach is transferable to other targets (e.g. other cancer antigens) for CAR therapy. In one embodiment, the CD247-KI CAR expressing immune cell targets HER-2. The CD247-KI CAR expressing immune cells targeting HER-2 provide the significant benefits comparable to the TRAC-KI HER2-CAR T cells or CAR T cells overexpressing the CAR from the hAAVS1 safe harbor locus via an EF1alpha-promoter or CMV-promoter driven expression cassette (refer to Example 15). CD247-KI HER2-CAR T cells display reduced exhaustion after typical CAR-T expansion, reduced differentiation and massively reduced activation-induced cell death. CD247-KI HER2 CAR T cells remained viable while AAVS1-KI and TRAC-KI CAR T cells started to undergo apoptosis.

In some embodiments, the targeting sequence comprises an identical sequence to a recognition site of the endogenous DNA sequence into which integration is intended, preferably selected from the group consisting of a homology arm, a guide RNA target site, a restriction enzyme recognition site, a ZFN recognition site, a ZFN cleaving site, a TALEN DNA-binding site, a recombinase recognition site, an integrase site and/or a homing nuclease recognition site.

In certain embodiments, the targeting sequence, termed “homology arm”, comprises at least one sequence complementary to an endogenous genomic sequence of the target cell. In one embodiment, the targeting sequence of the second sequence region configured for integration into a host genome comprises a homology arm complementary to an endogenous integration site, described above, preferably complementary to a site within the CD3 zeta/CD247 gene.

In some embodiments, the nucleic acid construct comprises in 5′ to 3′ order: a first vector DNA sequence, a left homology arm, a nucleic acid sequence encoding a self-cleaving peptide, the nucleic acid sequence encoding the CD3 zeta-deficient CAR fragment, a right homology arm, and a second vector DNA sequence. In certain embodiments, the first and the second vector DNA sequence can also be a first and second viral sequence (refer to Example 1). In some embodiments, no vector sequence is required or used.

In some embodiments, the nucleic acid construct comprises or consists of, in 5′ to 3′ order: a left homology arm, a nucleic acid sequence encoding a self-cleaving peptide, the nucleic acid sequence encoding the CD3 zeta-deficient CAR fragment, and a right homology arm.

In certain embodiments, a gene editing system for introducing the CD3 zeta deficient CAR fragment into an immune cell comprises:
i. a nucleic acid sequence encoding a CD3 zeta deficient CAR fragment,
ii. a nucleic acid sequence encoding or comprising a protein separation site, and
iii. a nucleic acid sequence encoding a homologous recombination system suitable for targeted integration of the nucleic acid sequence at a target site within the genome of the cell,
whereby the homologous recombination system integrates the construct at a site within the CD3 zeta/CD247 gene of said cell.

In one embodiment, the immune cells are preferably co-electroporated with a guide RNA, e.g. a sgRNA, targeting the CD3 zeta/CD247 gene, wherein the second nucleic acid sequence region of the construct comprises:
i) a left homology arm upstream of the CAR fragment at the 5′ end of the construct complementary to a first target sequence within the CD3 zeta/CD247 gene and
ii) a right homology arm downstream of the CAR fragment at the 3′ end of the construct complementary to a second target region within the CD3 zeta/CD247 gene.

In one embodiment, NK cells are preferably co-electroporated with the guide RNA targeting the CD3 zeta/CD247 gene. Guide RNA sequence is critical for a targeted genetic disruption. The guide RNA disclosed herein (refer to Example 5) provides beneficial effects to the targeted genetic disruption by guiding a specific cleavage. Targeted disruption of CD3 zeta/CD247 gene leads to correct integration of the CAR fragment by gene fusion, and results in sufficient translation of the functional CD3 zeta/CAR fusion protein. Such functional CD3 zeta/CAR fusion protein is advantageous as it is efficiently transported to and anchored into the plasma membrane with the CAR domain being functionally presented at cell surface and CD3 zeta element intracellular for inducing cell signaling. The beneficial effects provided by the CD247-KI approach using the guide RNA disclosed herein comprises less CAR cell exhaustion, reduced differentiation, massively reduced activation-induced cell death, and lower risks for side effects of CAR therapy, particularly using primary and immortalized NK cells.

The present invention therefore provides guide RNA sequence for targeting the CD3 zeta/CD247 gene that can be also applied for NK cells, and for reducing the side effects of CAR therapy and CAR cell product exhaustion, by enabling spatial, temporal, and persevering tumor-specific effects, achieved preferably by systemic administration of the cells, but exerted in a tissue specific manner due to CAR expression under the appropriate conditions.

In some embodiments, a homology directed repair (HDR) template is a nucleic acid molecule, preferably a double strand DNA, also used in the homology directed repair gene editing based technique. In certain embodiments of the method for generating a genetically modified immune cell, the double strand DNA repair template is a homology directed repair (HDR) template.

In some embodiments, a homology directed repair (HDR) template is generated as described in Roth et al. (2018 Nature).

In one embodiment, the invention provides means to insert the transgene into a cell using the homology-directed repair (HDR) method. In one embodiment, the HDR dsDNA template together with CRISPR-Cas9 ribonucleoprotein (Cas9) complexed with the single guide RNA (sgRNA) is transferred into the cell by means of co-electroporation (refer to Examples 1 and 5) Genome editing through HDR is advantageous for the specific and efficient and viral vector free insertion of large transgenes into target cells. It is advantageous that the viability and cell function of the transfected cells is maintained and allows a high yield of genetically modified cells for clinical application (refer to Example 6).

A further aspect of the invention relates to a genetically modified human cell, comprising an exogenous nucleic acid sequence region encoding a chimeric antigen receptor (CAR) fragment, without a sequence encoding a functional intracellular domain of a CD3 zeta protein, integrated in-frame into an endogenous CD3 zeta gene.

In some embodiments, the genetically modified cell is a product of modifying a cell with the construct as described herein. Features disclosed with respect to the construct or the method described above are applicable to the genetically modified cell, and vice versa.

In some embodiments, the genetically modified cell expresses a chimeric antigen receptor (CAR) as an in-frame fusion protein comprising the CAR fragment and an intracellular domain of the endogenous CD3 zeta gene, said intracellular domain comprising one or more immunoreceptor tyrosine-based activation motifs (ITAMs).

As described above, CD3 zeta protein comprises an extracellular and a transmembrane domain followed by an intracellular domain comprising three ITAMs. For a functional CAR in genetically modified immune cell, at least one ITAM is required, so that for choosing the integration site for the fusion of the CAR transgene into the CD3 zeta/CD247 gene, a site within the CD3 zeta/CD247 gene is preferably selected which is upstream of at least one ITAM, more preferably three ITAMs. A skilled person is capable of identifying a sequence encoding an ITAM domain and a suitable site for an in-frame integration for CAR/CD3 zeta fusion.

In one embodiment of a genetically modified immune cell, at least the first nucleic acid sequence region encoding the CAR fragment and the endogenous CD3 zeta sequence are fused to enable a polycistronic mRNA comprising coding regions for the polypeptide sequences of the CAR fragment and the endogenous CD3 zeta, wherein an amino acid sequence comprising a protein separation site, e.g. a peptide cleavage site, is positioned upstream of the CAR polypeptide.

In one embodiment of a genetically modified immune cell, the construct can additionally comprise sequences, e.g. encoding CAR regulating, immune-stimulating and/or therapeutic proteins, as described above, fused to said CAR fragment and configured to encode a polycistronic mRNA, wherein an amino acid sequence comprising a protein separation site, e.g. a peptide cleavage site, is disposed between the CAR polypeptide and each additional polypeptide.

In one embodiment of the genetically modified immune cell, a first nucleic acid encoding a CD3 zeta deficient CAR fragment directly follows a third nucleic acid region, providing a protein separation site. The spliceosome of the cell is capable of correctly splicing a transcript consisting of endogenous CD3 zeta sequences fused to said CD3 zeta deficient CAR fragment and said protein separation site, so that an open reading frame for the exogenous CAR fragment followed by at least one endogenous CD3 zeta ITAMs is maintained for translation. Upstream of the CAR fragment, an exogenous protein separation site follows endogenous CD3 zeta sequences. The protein separation site, preferably a P2A peptide cleavage site, is suitable to separate the CAR fragment polypeptide fused with the CD3 zeta ITAM polypeptides from the unwanted endogenous CD3 zeta segments during or after translation.

In one embodiment of the genetically modified immune cell, said the fusion CAR polypeptide is functional and presented on the surface of said immune cell.

In one embodiment of the genetically modified immune cell, the chimeric antigen receptor (CAR)-CD3 zeta fusion gene is expressed under the control of an endogenous CD3 zeta promoter and an endogenous CD3 zeta expression termination signal.

In one embodiment of the genetically modified immune cell, the chimeric antigen receptor (CAR)-CD3 zeta fusion gene is expressed under the control of an endogenous CD3 zeta promoter and wherein expression of the chimeric antigen receptor (CAR) is terminated by an endogenous CD3 zeta gene expression termination signal and an endogenous poly-A signal.

In some embodiments of the genetically modified cell, the CAR transgene is fused to an endogenous CD3 zeta/CD247 gene, wherein the fusion site is downstream of the endogenous CD3 zeta/CD247 gene promoter, so that said fusion CAR is under control of the promoter and expression termination signal of the endogenous CD3 zeta/CD247 gene.

In some embodiments, the CAR transgene fused into the CD3 zeta/CD247 gene is or may be termed a CAR-CD3 zeta fusion gene and its corresponding transcript CAR-CD3 zeta fusion mRNA, wherein a polypeptide translated from a CAR-CD3 zeta fusion mRNA is or may be termed a CAR-CD3 zeta fusion protein, or fusion CAR.

The expert would not expect that the endogenous expression regulation of a CAR/CD3 zeta fusion provides such a beneficial effect on anti-tumor activity, endurance, expansion of the CAR cell product, especially as, in some embodiments, the expression of the CAR/CD3 zeta fusion protein is reduced compared to prior art CARs.

The control of gene expression under an endogenous promoter of a TCR-complex component offers the advantage of regulated gene expression as required by the cell to become active against a pathogenic cell, preferably a tumor cell, depending on tumor signals and signals that activate an immune cell.

In some embodiments, the endogenous expression and/or function of a TCR complex, preferably the expression and/or function of a TCR alpha and/or beta chain, or TCR alpha, beta, delta and/or gamma chain, is disturbed (perturbed, disrupted and/or inhibited) in the genetically modified cell.

In some embodiments of the genetically modified cell, the endogenous the expression and/or function of a TCR alpha and/or beta chain is disrupted

In some embodiments, integration of the construct disrupts or reduces expression of a protein selected from the group consisting of CD3 gamma chain, CD3 delta chain, CD3 epsilon chain, and CD3 zeta chain (refer to Examples 2 and 3).

In some embodiments, an immune cell is provided in which the fusion CAR is expressed at the surface of the cell, and wherein the expression of a functional TCR complex and presentation at the surface of the cell is reduced and/or absent.

In some embodiments, the genetically modified cell is an immune cell, preferably selected from the group consisting of a T lymphocyte, a T regulatory (Treg) cell, natural killer (NK) cell, a natural killer T (NKT) cell, cytokine-induced killer cell (CIK), an immortalized immune cell including NKT, NK-92 and YT cells, or a cell derived from peripheral human blood, human cord blood, bone marrow stem cells or induced pluripotent stem cells (iPSC), wherein the T lymphocyte is preferably a CD4 or CD8 T-cell, more preferably a cytotoxic T lymphocyte or a T helper cell. As shown in Example 13, the CD247-KI approach provides mean for reprogramming of different cell types for adoptive T cell transfer.

In some embodiments of the genetically modified cell, the cell is a T lymphocyte, wherein the T lymphocyte is preferably a CD4 or CD8 T-cell, more preferably a cytotoxic T lymphocyte or a T helper cell. In some embodiments of the genetically modified cell, the cell is a cytokine-induced killer cell (CIK). In some embodiments of the genetically modified cell, the cell is a T regulatory (Treg) cell. In some embodiments of the genetically modified cell, the cell is a natural killer (NK) cell. In some embodiments of the genetically modified cell, the cell is a natural killer T (NKT).

In some embodiments of the genetically modified cell, the cell is an immortalized immune cell including NKT, NK-92 and YT cells. In some embodiments of the genetically modified cell, the cell is a cell derived from peripheral human blood, bone marrow stem cells or human cord blood. In some embodiments of the genetically modified cell, the cell is a cell derived from induced pluripotent stem cells (iPSC).

In adoptive cell transfer, T-cell based cytotoxic reactions are used to attack cancer cells. T cells that have a natural or genetically engineered response to a patient's cancer are generated in vitro and then transferred back to the cancer patient. Such T cells can be stimulated to proliferate in vitro by high concentrations of IL-2 and/or other cytokines (such as IL-7 and IL-15), anti-CD3 and anti-CD28 coated microbeads. The present invention therefore encompasses, in some embodiments, adoptive cell transfer in combination with the administration of the CAR expressing cells described herein.

The expression of stimulatory cytokines enhances the activation of T cells only locally, preferably within or near the tumor, and subsequently leads to a memory effector cell phenotype, thereby prolonging the therapeutic effect of the treatment. A surprisingly reduced cytokine production after antigen-engagement by the CD3 zeta truncated CAR of the invention integrated into the CD3 zeta/CD247 gene is additionally advantageous compared to the prior art CARs inserted into the TRAC locus. Furthermore, in some embodiments, of particular advantage for the CAR transgene is a reduced CAR expression and CAR surface density, leading to reduced toxicity, lower risk for over-activation or on-target off-tumor toxicity such as cytokine release syndrome or ICANS. (refer to Example 10).

In embodiments of the genetically modified immune cell, the immune cell is a T cell, an NK cell or a Treg cell. As shown in Example 12, integration of the CD3 zeta truncated CAR into the CD3 zeta/CD247 gene can be used to generate CAR positive CD4+ regulatory T cells.

In one embodiment, the CAR expressing cells express more than one immune stimulating and/or immune suppression defeating gene and/or an inducible suicide gene allowing said cells to be destroyed. A suicide gene, as non-limiting embodiments, is one that codes for the thymidine kinase of an alpha herpesvirus (HHV1-3), the bacterial gene cytosine deaminase, which can convert 5-fluorocytosine into the highly toxic compound 5-fluorouracil, and inducible caspase-9 or caspase-8 or cytosine deaminase. Inducible caspase-9 can be activated by a specific chemical inducer of dimerization (CID). Suicide genes may also be polypeptides that are expressed on the cell surface and can make the cells sensitive to therapeutic monoclonal antibodies. The suicide gene expression may be inducible, for example by doxycyclin adapted to human cells.

A further aspect of the invention relates to a genetically modified cell for use as a medicament.

In some embodiments, the cell is for use in the treatment of a medical disorder associated with the presence of pathogenic cells expressing oncogenes, such as cancer cells, more preferably cancer stem cells of solid and hematologic malignancies.

In some embodiments, the cell is for use in the treatment of an autoimmune disease or a graft versus host disease.

As such, the CAR of this invention represents a remarkable and beneficial approach to the treatment of various diseases described herein, susceptible to targeted activity of a modified immune cell. The minimal (if any) adverse side effects due to the selectivity and specificity of the expression regulation also represent a beneficial and surprising aspect of the present invention.

Especially in patients who have developed resistance to other solid or liquid tumor treatments, the present invention represents a promising approach to the eradication of malignant tumors. As shown in Example 11, a CAR integration into CD3 zeta/CD247 abolishes allo-reactivity of CAR T cells similar to TRAC inserted CAR T cells.

In some embodiments it is advantageous, that the CAR construct in the present invention can be used in therapy of solid tumors and/or liquid tumors, i.e. leukaemia or lymphoma. In some embodiments, the genetically modified cell is used as a medicament, wherein the disorder to be treated with said immune cell is selected from a group of cancers as described herein, preferably consisting of prostate cancer, breast cancer, melanoma, colon carcinoma, acute lymphocytic leukemia (ALL), and Non-Hodgkin lymphoma (NHL).

The CD247 KI approach was tested in vivo using CD247 KI CD19 CAR T cells in a xenograft pre-B ALL mouse model in immunodeficient NRG mice. Significant leukaemia control, advantageous to the prior art, was demonstrated in a xenograft pre-B ALL mouse model with CD247-KI CD19-CAR T cells (refer to Example 14). This substantiates the benefit and high efficacy, safety, efficacy as well as functionality of the CD247 KI CAR cell strategy disclosed herein.

The various aspects of the invention are bound by the surprising and beneficial concept of local immune system stimulation in an anti-tumor immune response, either by the innate immune system or by combined immunotherapies. It was unexpected that a TCR deficient CAR-T cell would express and present a functional, transgenic CAR/CD3 zeta fusion protein, as shown in the examples 2 to 5, exhibiting such an effective anti-tumor effect. Furthermore, it was unexpected that a CAR-NK cell and a CAR-Treg cell would effectively express and present the transgenic CAR/CD3 zeta fusion protein, as shown in the examples.

The present invention enables a surprising and advantageous endogenous CAR expression rate beneficial for an anti-tumor effect via the CAR/CD3 zeta fusion protein in CAR-T cell or CAR-NK cell as described herein. The endogenously regulated CAR expression (Example 3) combined with improved cell expansion and endurance as described herein supports an anti-tumor immune response and leads to reduction in tumor size and/or growth, and shows a distinct reduction in and/or avoidance of the side effects produced by overexpression of CARs and TCR complex expression in an allogeneic condition (refer to Example 11). Side effects such as GvHD, ICANS (immune effector cell-associated neurotoxicity syndrome), cytokine release syndrome, immune response to recombinant proteins, tumor escape, nausea and vomiting, sores in the mouth or on the lips, diarrhea, drowsiness, allergic reactions, fever or chills, hives, itching, headache, coughing, shortness of breath, or swelling of the face, tongue, or throat, may be avoided by the approach described herein.

The present invention therefore provides means for reducing the side effects of CAR therapy and CAR cell product exhaustion, by enabling spatial, temporal, and persevering tumor-specific effects, achieved preferably by systemic administration of the cells, but exerted in a tissue specific manner due to CAR expression under the appropriate conditions.

As a further positive effect in the avoidance or reduction of side effects in CAR therapy, a significantly lower cytokine production is observed after antigen exposure by a CD3 zeta deficient CAR integrated in the CD247 gene, compared to a prior art CAR.

In one embodiment, the genetically modified cell is a CD4+ and/or CD8+ T cell, preferably a mixture of CD4+ and CD8+ T cells. These T cell populations, and preferably the composition comprising both CD4+ and CD8+ transformed cells, show particularly effective cytolytic activity against various solid or liquid tumors.

In one embodiment, the modified cell comprises CD4+ and CD8+ T cells, preferably in a ration of 1:10 to 10:1, more preferably in a ratio of 5:1 to 1:5, 2:1 to 1:2 or 1:1. Administration of anti-tumor-antigen-directed modified CAR-T cells expressing the CAR fragment described herein, for example anti-CD19 CAR fragment or anti-HER2 CAR fragment, at the ratios mentioned, preferably at a 1:1 CD4+/CD8+ ratio, lead to beneficial characteristics during treatment of the diseases mentioned herein, for example these ratios lead to improved therapeutic response and reduced toxicity (refer to Examples 8 and 11).

In one embodiment, the genetically modified cell carrying a nucleic acid construct encoding a CD3 zeta-deficient CAR fragment in another gene locus than CD3 zeta/CD247 provides no or not therapeutically relevant cytotoxicity levels.

In one embodiment, the genetically modified cell is allogeneic with respect to a patient, preferably an allogeneic T-cell, NK-cell or Treg.

In one embodiment, the genetically modified cell is an allogeneic NK-cell with respect to a patient.

In one embodiment, the genetically modified cell is an allogeneic Treg with respect to a patient.

In one embodiment, the genetically modified cell is an allogeneic TCR gamma/delta T-celll with respect to a patient.

In one embodiment, the genetically modified cell is autologous with respect to a patient, preferably an autologous NK-cell.

In one embodiment, the genetically modified cell is an autologous NK-cell with respect to a patient.

In one embodiment, the genetically modified cell is an autologous Treg with respect to a patient.

In one embodiment, the genetically modified cell is an autologous TCR gamma/delta T-celll with respect to a patient.

In some embodiments, cells can be obtained from a subject different from the intended patient, therefore allogeneic cells. As used herein, a cell is “allogeneic” with respect to a subject if it or one of its progenitor cells is derived from another subject of the same species. As used herein, a cell is “autologous” with respect to a subject if it or its progenitor cells originate from the same subject.

For the use of “off-the-shelf” allogeneic or autologous CAR-T cells or CAR-NK cells, preferably CAR-NK cells, the inventors developed a method of engineering a tumor-antigen expressing CAR-T cells or CAR-NK cells that exhibit fewer unwanted side effects, if any, than allogeneic CAR-expressing cells to date.

Allogeneic “off the shelf” CAR-T cell products, as described in the present invention, can be produced from cells of healthy donors in stock and are applicable to a large patient population, depending on the indication and/or tumor-specific antigen. The means provided by the invention enable to reduce the cost of individual doses of CAR-T cell products and failures in the production of CAR-T cells. In particular, the start of therapy is accelerated to the benefit of the patient (refer to Example 12).

The most serious risk associated with the use of allogeneic T-cell products is an often-fatal acute graft-versus-host disease (GvHD), whereby the donor's (CAR-modified) T-cells recognize and attack the patient's tissue as “foreign” through their endogenous TCR. Due to the beneficial properties of the cells of the invention these risks are reduced compared to known approaches.

Transplantation of autologous or allogeneic cells expressing the fusion CAR of the invention is feasible. For example, when reintroduced back to patients after autologous cell transplantation, the T cells modified with the CAR fragment of the invention as described herein may recognize and kill tumor cells (refer to Example 9).

Employing CAR-NK cells allows to reduce risks for a graft versus host disease in an allogeneic cell context. The CAR-NK cell product can be preferably used as allogeneic “off the shelf” product. The present invention enables a surprisingly safe and advantageous use of allogeneic immune cells, in particular of allogeneic CAR-T cells, by their beneficial loss of TCR presentation on the cell surface, as described above, and the intracellular, tumor-dependent regulated expression of the CAR/CD3 zeta fusion protein in the CAR-T cell or CAR-NK cell, as described here.

The invention further relates to a chimeric antigen receptor (CAR) polypeptide expressed from a genetically modified cell as described herein, comprising a chimeric antigen receptor (CAR) as an in-frame fusion protein comprising an exogenous CAR fragment without a sequence encoding a functional intracellular domain of a CD3 zeta protein, and an intracellular domain of the endogenous CD3 zeta/CD247 gene, said intracellular domain comprising one or more immunoreceptor tyrosine-based activation motifs (ITAMs).

In one embodiment, the CAR fragment comprises:
a CAR signal sequence, preferably according to SEQ ID NO 4, or a sequence with at least 80% sequence identity to SEQ ID NO 4, and
a single chain variable fragment, for example a murine single chain variable fragment specific to the human CD19 antigen, preferably according to SEQ ID NO 5 and 7 or according to SEQ ID NO 59, or a sequence with at least 80% sequence identity to SEQ ID NO 5 and 7, or to SEQ ID NO 59, and
an immunoglobulin heavy chain constant region or a spacer of a CAR, preferably according to SEQ ID NO 8-10, or a sequence with at least 80% sequence identity to SEQ ID NO 8-10; and
a CD28 signalling domain, preferably according to SEQ ID NO 11 and 12 or a sequence with at least 80% sequence identity to SEQ ID NO 11 and 12; wherein the CD28 signaling domain comprises a transmembrane domain, preferably according to SEQ ID NO 11, or a sequence with at least 80% sequence identity to SEQ ID NO 11.

In some embodiments, the construct may further comprise
a truncated CD3 zeta signaling domain including a stop signal for protein translation, preferably according to SEQ ID NO 21, or a sequence with at least 80% sequence identity to SEQ ID NO 21, or
a truncated CD3 zeta signaling domain lacking a stop signal for protein translation, preferably according to SEQ ID NO 18, or a sequence with at least 80% sequence identity to SEQ ID NO 18, or

In further embodiments, the invention relates to a CAR polypeptide that comprises one or more exogenous linker, an exogenous spacer, an exogenous transmembrane, and exogenous and endogenous signaling domains. In one embodiment, the CAR comprises an intracellular domain, which comprises an exogenous co-stimulatory domain and an endogenous signaling (activation) domain, wherein the endogenous signaling domain is preferably a CD3 zeta domain.

The exchange of exogenous signaling domains meets the demands for either a strong and rapid effector phase (CD28 co-stimulatory domain), or a long-lasting relapse control as secured by a T cell memory population (4-1 BB signaling domain). As demonstrated herein, the various signaling domains may be exchanged in multiple configurations, providing a CAR with flexibility with respect to its design without loss of the advantageous binding properties.

Due to the variants (by adding alternative components) employed as the linker, spacer, transmembrane and intracellular domains, it becomes apparent that the various components may be exchanged at required by the skilled person, and the tumor-antigen binding properties may be maintained, thereby maintaining the desired biological effects.

It is also possible to exchange the intracellular ITAM comprising protein domain as required, for example the CD3 chains epsilon, gamma and delta are known to a skilled person.

In some embodiments, the use of a “TCR complex specific” promoter may show a synergistic effect in combination with the CAR-T cell or CAR-NK cell activation signal, cell specificity, and cell expansion with respect to reduction of unwanted systemic side effects. The CAR-T cell or CAR-NK of the present invention migrates towards inflammatory, in particular tumor, tissue. The use of a promoter for the expression of the cytokine that is preferentially expressed under conditions of inflammation or of being present in tumor tissue further enhances the reduction in systemic expression in a synergistic manner, thereby providing surprising benefits (refer to Example 7).

The invention further relates to a pharmaceutical composition comprising a genetically modified cell as described herein and a pharmaceutically acceptable carrier.

Treatment with the genetically modified cell according to the invention can in some embodiments be combined with one or more anti-cancer treatments or medicaments, preferably selected from the group of antibody therapy, vaccines, oncolytic viral therapy, chemotherapy, radiation therapy, cytokine therapy, dendritic cell therapy, gene therapy, hormone therapy, laser light therapy, immune suppression or transplantation.

The invention relates further to methods of treatment of the medical conditions described herein, comprising typically the administration of a therapeutically effective amount of the CD3 zeta deficient CAR fragment complemented with an endogenous CD3 zeta domain, or immune cell expressing said CAR, to a patient in need of said treatment.

The invention further relates to a pharmaceutical composition comprising a genetically modified cell according the invention described herein and a pharmaceutically acceptable carrier. The composition described herein may be administered to a patient subcutaneously, intradermally, intratumorally, intranodally, intramedullary, intramuscularly, by intravenous or intralymphatic injection, or intraperitoneally.

All features described in the present specification may be employed to define any other embodiment or aspect of the invention, for example, features used to describe the cell may be used to describe the construct, and vice versa. Similarly, features used to describe the methods of the invention may be used to describe the cells or constructs, and vice versa.

### DETAILED DESCRIPTION OF THE INVENTION

An important function of the immune system is to recognize and eliminate tumors. Tumor antigens are either specifically expressed on tumor cells and not found on non-pathogenic cells or abnormally expressed, e.g. at least twice above the level found in non-pathogenic cells, such as CD19. Antigens, specifically found on tumor cells, may appear foreign to the immune system and their presence may cause the immune cells to attack the transformed tumor cells.

The response of the immune system to tumors is to destroy abnormal cells with the help of killer T cells, natural killer (NK) cell, sometimes with helper T cells. Tumor antigens are presented on MHC class I molecules in a similar way to viral antigens. This enables killer T cells (CD8+ T cells) to recognize the tumor cell as abnormal. MHC class II molecules presented tumor antigens stimulate T helper cells (CD4+ T cells). NK cells also kill tumor cells in a similar way, especially if the tumor cells have fewer MHC class I molecules on their surface than normal; this is a common phenomenon in tumors. Some tumor cells also release products that inhibit the immune response, for example by secreting the cytokine TGF-β, which suppresses the activity of macrophages and lymphocytes. Cytokine-induced killer cells (CIK) are a group of immune effector cells that exhibit a hybrid T- and NK-like phenotype.

The present invention, as described herein, therefore provides means for enabling and/or enhancing an anti-tumor immune response by the expression of a chimeric antigen receptor (CAR) directed against a tumor antigen, e.g. CD19, in a genetically modified immune cell, e.g. T lymphocytes, cytokine-induced killer cell (CIK), NK cell. The expression of said CAR is controlled by a specific endogenous promoter, while abandoning TCR complexes in said genetically modified immune cell, as described herein.

Immunotherapy in the context of the present invention is to be understood to comprise any therapeutic agent employing the immune system for cancer treatment. Immunotherapy takes advantage of the fact that cancer cells have subtly different molecules on their surface, which can be recognized by the immune system. Immunotherapy encompasses, without limitation, cellular and antibody therapy. Cellular therapies typically involve the administration of immune cells isolated from the blood or from a tumor of the patient. Immune cells directed towards the tumor to be treated are activated, cultured and returned to the patient where the immune cells attack the cancer. Cell types that can be used in this way are, without limitation, natural killer cells, lymphokine-activated killer cells, cytotoxic T cells, and dendritic cells. Dendritic cell therapy provokes anti-tumor responses by causing dendritic cells to present tumor antigens. Dendritic cells present antigens to lymphocytes, which activates them, priming them to kill other cells that present the antigen.

The present invention therefore provides a strategy for immune cell therapy that uses gene editing methods to preferably integrate one or more therapeutic transgenes under the control of at least one endogenous promoters and expression termination signals enabling cell-specific and endogenously controlled expression in therapeutic immune cells. The various aspects and embodiments of the invention, in addition to their advantages over the prior art, are described above.

Chimeric Antigen Receptors:

In various embodiments, genetically modified receptors, which redirect the cytotoxicity of immune effector cells are provided. These genetically engineered receptors are referred to herein as chimeric antigen receptors (CARs). CARs are molecules that combine antibody-based specificity for a desired antigen (e.g., CD19) with a T cell receptor-activating intracellular domain to generate a chimeric protein that exhibits a specific anti-gene cellular immune activity, e.g. against CD19. As used herein, the term, “chimeric,” describes being composed of parts of different proteins or DNAs from different origins.

According to the present invention, a CAR comprises an extracellular antigen-binding domain, comprising an antibody or antibody fragment that binds a target antigen, a transmembrane domain, and an intracellular domain. CARs are typically described as comprising an extracellular ectodomain (antigen-binding domain) derived from an antibody and an endodomain comprising signaling modules originally derived from T cell signaling proteins.

In a preferred embodiment, the ectodomain preferably comprises variable regions from the heavy and light chains of an immunoglobulin configured as a single-chain variable fragment (scFv). The scFv is preferably attached to a hinge region that provides flexibility and transduces signals through an anchoring transmembrane moiety to an intracellular signaling domain. The transmembrane domains originate preferably from either CD8a or CD28. In the first generation of CARs the signaling domain consists of the zeta chain of the TCR complex. The term “generation” refers to the structure of the intracellular signaling domains. Second generation CARs are equipped with a single costimulatory domain originated from CD28 or 4-1 BB. Third generation CARs already include two costimulatory domains, e.g. CD28, 4-1 BB, ICOS or OX40, CD3 zeta. Fourth generation CARs additionally deliver immune stimulatory proteins and/or checkpoint regulatory proteins. The fifth CAR generation incorporates control elements for the regulation of CAR expression, which allows to induce and/or completely switch off the CAR expression or CAR expressing cells, e.g. by suicide genes. The present invention preferably relates to a third, fourth or fifth generation CAR.

The methods of the present invention comprise the administration of cells designed to express a CAR. In some embodiments, the ligand or extracellular ligand binding domain is selected so that the cell expressing the CAR is targeted to a cancer cell or tumor. Any cancer antigen can be a tumor antigen and any tumor antigen can be a caner antigen. The extracellular antigen-binding domain of a CAR is usually derived from a monoclonal antibody (mAb) or from receptors or their ligands.

CARs contemplated herein, comprise an extracellular domain (also referred to as a binding domain or antigen-binding domain) that binds to a preferred target protein, a transmembrane domain, and an intracellular domain, or intracellular signaling domain. “Target protein” and “target antigen” are used interchangeably. In one embodiment, the preferred target protein is CD19. Engagement of the anti-CD19 antigen binding domain of the CAR with CD19 on the surface of a target cell results in clustering of the CAR and delivers an activation stimulus to the CAR-containing cell.

In various embodiments, a CAR comprises an extracellular binding domain that comprises a humanized CD19-specific binding domain; a transmembrane domain; one or more intracellular signaling domains. In particular embodiments, a CAR comprises an extracellular binding domain that comprises a humanized anti-CD19 antigen binding fragment thereof; one or more spacer domains; a transmembrane domain; one or more intracellular signaling domains. The “extracellular antigen-binding domain” or “extracellular binding domain” are used interchangeably and provide a CAR with the ability to specifically bind to the target antigen of interest, CD19. The binding domain may be derived either from a natural, synthetic, semisynthetic, or recombinant source. Preferred in some embodiments are scFv domains or VHH (variable heavy chain only) domain fragments.

“Specific binding” is to be understood as via one skilled in the art, whereby the skilled person is clearly aware of various experimental procedures that can be used to test binding and binding specificity. Methods for determining equilibrium association or equilibrium dissociation constants are known in the art. Some cross-reaction or background binding may be inevitable in many protein-protein interactions; this is not to detract from the “specificity” of the binding between CAR and epitope. By way of example, “specific binding” describes binding of an e.g. anti-CD19 antibody or antigen binding fragment thereof (or a CAR comprising the same) to CD19 at greater binding affinity than background binding. The term “directed against” is also applicable when considering the term “specificity” in understanding the interaction between antibody and epitope.

An “antigen (Ag)” refers to a compound, composition, or substance that can stimulate the production of antibodies or a T cell response in an animal. In particular embodiments, the target antigen is an epitope of a CD19 polypeptide. An “epitope” refers to the region of an antigen to which a binding agent binds. Epitopes can be formed both from contiguous amino acids or noncontiguous amino acids juxtaposed by tertiary folding of a protein. Any suitable antigen can be selected depending on the type of cancer.

“Single-chain Fv” or “scFv” antibody fragments comprise the VH and VL domains of an antibody, wherein these domains are present in a single polypeptide chain and in either orientation {e.g., VL-VH or VH-VL). Generally, the scFv polypeptide further comprises a polypeptide linker between the VH and VL domains which enables the scFv to form the desired structure for antigen binding. In preferred embodiments, a CAR contemplated herein comprises antigen-specific binding domain that is a scFv and may be a murine, human or humanized scFv. Single chain antibodies may be cloned from the V region genes of a hybridoma specific for a desired target.

In particular embodiments, the antigen-specific binding domain that is a humanized scFv that binds a human CD19 polypeptide. An illustrative example of a variable heavy chain that is suitable for constructing anti-CD19 CARs contemplated herein include, but are not limited to the amino acid sequence set forth in SEQ ID NO: 5-7. An illustrative example of a variable light chain that is suitable for constructing anti-CD19 CARs contemplated herein include, but is not limited to the amino acid sequence set forth in SEQ ID NO: 5-7.

Antibodies and Antibody Fragments:

The CAR comprises an extracellular antigen-binding domain, comprising for example an antibody or antibody fragment that binds CD19 polypeptide. Antibodies or antibody fragments of the invention therefore include, but are not limited to polyclonal, monoclonal, bispecific, human, humanized or chimeric antibodies, single chain fragments (scFv), single variable fragments (ssFv), single domain antibodies (such as VHH fragments from nanobodies), Fab fragments, F(ab′)2 fragments, fragments produced by a Fab expression library, anti-idiotypic antibodies and epitope-binding fragments or combinations thereof of any of the above, provided that they retain similar binding properties of the CAR described herein, preferably comprising the corresponding CDRs, or VH and VL regions as described herein. Also mini-antibodies and multivalent antibodies such as diabodies, triabodies, tetravalent antibodies and peptabodies can be used in a method of the invention. The immunoglobulin molecules of the invention can be of any class (i.e. IgG, IgE, IgM, IgD and IgA) or subclass of immunoglobulin molecules. Thus, the term antibody, as used herein, also includes antibodies and antibody fragments comprised by the CAR of the invention, either produced by the modification of whole antibodies or synthesized de novo using recombinant DNA methodologies.

As used herein, an “antibody” generally refers to a protein consisting of one or more polypeptides substantially encoded by immunoglobulin genes or fragments of immunoglobulin genes. Where the term “antibody” is used, the term “antibody fragment” may also be considered to be referred to. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. The basic immunoglobulin (antibody) structural unit is known to comprise a tetramer or dimer. Each tetramer is composed of two identical pairs of polypeptide chains, each pair having one “light” (L) (about 25 kD) and one “heavy” (H) chain (about 50-70 kD). The N-terminus of each chain defines a variable region of about 100 to 110 or more amino acids, primarily responsible for antigen recognition. The terms “variable light chain” and “variable heavy chain” refer to these variable regions of the light and heavy chains respectively. Optionally, the antibody or the immunological portion of the antibody, can be chemically conjugated to, or expressed as, a fusion protein with other proteins.

The CARs of the invention are intended to bind against mammalian, in particular human, protein targets. The use of protein names may correspond to either mouse or human versions of a protein.

Affinities of binding domain polypeptides and CAR proteins according to the present disclosure can be readily determined using conventional techniques, e.g., by competitive ELISA (enzyme-linked immunosorbent assay), or by binding association, or displacement assays using labeled ligands, or using a surface-plasmon resonance device such as the Biacore.

A variable region of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies.

Additional Components of the CAR

In certain embodiments, the CARs contemplated herein may comprise linker residues between the various domains, added for appropriate spacing and conformation of the molecule, for example a linker comprising an amino acid sequence that connects the VH and VL domains and provides a spacer function compatible with interaction of the two sub-binding domains so that the resulting polypeptide retains a specific binding affinity to the same target molecule as an antibody that comprises the same light and heavy chain variable regions. CARs contemplated herein, may comprise one, two, three, four, or five or more linkers. In particular embodiments, the length of a linker is about 1 to about 25 amino acids, about 5 to about 20 amino acids, or about 10 to about 20 amino acids, or any intervening length of amino acids. Illustrative examples of linkers include glycine polymers; glycine-serine polymers; glycine-alanine polymers; alanine-serine polymers; and other flexible linkers known in the art, such as the Whitlow linker. Glycine and glycine-serine polymers are relatively unstructured, and therefore may be able to serve as a neutral tether between domains of fusion proteins such as the CARs described herein. In particular embodiments, the binding domain of the CAR is followed by one or more “spacers” or “spacer polypeptides,” which refers to the region that moves the antigen binding domain away from the effector cell surface to enable proper cell/cell contact, antigen binding and activation. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region. In one embodiment, the spacer domain comprises the CH2 and CH3 domains of IgG1 or IgG4. In one embodiment the Fc-binding domain of such a spacer/hinge region is mutated in a manner that prevents binding of the CAR to Fc-receptors expressed on macrophages and other innate immune cells.

The binding domain of the CAR may in some embodiments be followed by one or more “hinge domains,” which play a role in positioning the antigen binding domain away from the effector cell surface to enable proper cell/cell contact, antigen binding and activation. A CAR may comprise one or more hinge domains between the binding domain and the transmembrane domain™. The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CARs described herein include the hinge region derived from the extracellular regions of type 1 membrane proteins such as CD8 alpha, CD4, CD28, PD1, CD 152, and CD7, which may be wild-type hinge regions from these molecules or may be altered. In another embodiment, the hinge domain comprises a PD1, CD 152, or CD8 alpha hinge region.

The “transmembrane domain” is the portion of the CAR that fuses the extracellular binding portion and intracellular signaling domain and anchors the CAR to the plasma membrane of the immune effector cell. The TM domain may be derived either from a natural, synthetic, semi-synthetic, or recombinant source. The TM domain may be derived from the alpha, beta or zeta chain of the T cell receptor, CD3s, O′3ζ, CD4, CD5, CD8 alpha, CD9, CD16, CD22, CD27, CD28, CD33, CD37, CD45, CD64, CD80, CD86, CD134, CD137, CD152, CD154, and PD1. In one embodiment, the CARs contemplated herein comprise a TM domain derived from CD8 alpha or CD28.

In particular embodiments, CARs contemplated herein comprise an intracellular signaling domain. An “intracellular signaling domain,” refers to the part of a CAR that participates in transducing the message of effective anti-CD19 CAR binding to a human CD19 polypeptide into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. The term “effector function” refers to a specialized function of an immune effector cell. Effector function of the T cell, for example, may be cytolytic activity or help or activity including the secretion of a cytokine. Thus, the term “intracellular signaling domain” refers to the portion of a protein which transduces the effector function signal and that directs the cell to perform a specialized function. CARs contemplated herein comprise one or more co-stimulatory signaling domains to enhance the efficacy, expansion and/or memory formation of T cells expressing CAR receptors. As used herein, the term, “co-stimulatory signaling domain” refers to an intracellular signaling domain of a co-stimulatory molecule. Co-stimulatory molecules are cell surface molecules other than antigen receptors or Fc receptors that provide a second signal required for efficient activation and function of T lymphocytes upon binding to an antigen. The costimulatory molecule is a cell surface molecule other than an antigen receptor or its ligand that contributes to an efficient immune response. Costimulatory molecules include MHC class I molecules, BTLA and Toll ligand receptors, OX40, CD27, CD28, CDS, ICAM-1, LFA-1 (CD11a/CD18), ICOS (CD278) and 4-1 BB (CD137). Including, but not limited to, further examples of such costimulatory molecules are CDS, ICAM-1, GITR, BAFFR, HVEM (LIGHTTR), SLAMF7, NKp80 (KLRF1), NKp44, NKp30, NKp46, CD160, CD19, CD4, CD8 alpha, CD8 beta, IL2Rbeta, IL2Rgamma, IL7Ralpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD 26), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a and ligands that specifically bind to CD83.

A costimulatory intracellular signaling domain can be the intracellular portion of a costimulatory molecule. In one embodiment, the CAR comprises an intracellular domain, which comprises a co-stimulatory domain and a signaling (activation) domain. The CAR construct may therefore include an intracellular signaling domain (CD3 zeta) of the native T cell receptor complex and one or more co-stimulatory domains that provide a second signal to stimulate full T cell activation. Co-stimulatory domains are thought to increase CAR-T cell cytokine production and facilitate T cell replication and T cell persistence. Co-stimulatory domains have also been shown to potentially prevent CAR-T cell exhaustion, increase T cell anti-tumor activity, and enhance survival of CAR-T cells in patients. As a non-limiting example, CAR constructs with the 4-1 BB co-stimulatory domain have been associated with gradual, sustained expansion and effector function, increased persistence, and enriched central memory cells (TCM) in the T cell subset composition in preclinical studies. 4-1 BB is a member of the tumor necrosis factor (TNF) superfamily, and it is an inducible glycoprotein receptor in vivo that is primarily expressed on antigen-activated CD4 and CD8 T cells. As a non-limiting example, CD28 is member of the immunoglobulin (Ig) superfamily. It is constitutively expressed on resting and activated CD4 and CD8 T cells and plays a critical role in T cell activation by stimulating the PI3K-AKT signal transduction pathway. In one embodiment, the intracellular domain comprises both 4-1 BB and CD28 co-stimulatory domains. Other co-stimulatory domains comprise ICOS and OX40 that can be combined with the CD3 zeta signaling (activation) domain.

In some non-limiting embodiments, an intracellular domain of a CAR may additionally comprise at least one co-stimulating signal domain. Such a co-stimulatory signal domain can result in increased activation of a T cell.

The polypeptide domains described herein may be useful in an anti-cancer CAR to mediate a desired function such as that of a signal peptide, antigen, binding domain, transmembrane domain, intracellular signal domain and/or co-stimulating domain. Potential desirable functions may comprise, but are not limited to, providing a signal peptide and/or transmembrane domain.

The cytokines described herein may relate to any mammalian cytokine corresponding to the cytokine named herein. Preferably, the cytokines relate to the human cytokines, or mouse cytokines. Cancer immunotherapy attempts to stimulate the immune system to reject and destroy tumors. Initially, immunotherapy treatments involved administration of cytokines such as “Interleukin”, as described herein.

Checkpoint inhibitors, also known as immune checkpoint modulators, are designed to lessen the effectiveness of checkpoint proteins. They may have a variety of mechanisms of action, but if effective, they enable the immune system to recognize other molecules on the surface of the cancer cells. The checkpoint inhibitor of the immune response is selected from the group consisting of: PD1, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGFR beta.

CAR-regulating proteins can also be proteins that induce or completely switch off a CAR expression or a cell expressing a CAR. In another embodiment, the CAR-T cells or CAR-NK cells additionally comprise one or more immune suppression defeating proteins and/or an inducible suicide gene that provokes CAR-T cell or CAR-NK cell death allowing their selective destruction. In some cases, it may be desirable to provide a safety mechanism that allows selective deletion of the administered genetically modified cells, as the manipulated cells can spread and persist for years after administration. Therefore, the method of the invention in some embodiments may include the transformation of preferably the T cells, NK cells or Tregs with a recombinant suicide gene. This recombinant suicide gene can be used to preferably reduce the risk of direct toxicity and/or uncontrolled proliferation of these T cells, NK cells or Tregs after administration to a subject. Suicide genes also enable the selective deletion of transformed cells in vivo. In particular, the suicide gene has the ability to convert a non-toxic prodrug into a cytotoxic drug or to express the toxic gene expression product. In other words, “suicide gene” is preferably a nucleic acid that codes for a product, whereby the product itself or in the presence of other compounds causes cell death.

TCR, CD3 and CD3 Zeta

A “T cell receptor” (TCR) is a heterodimer of the TCR alpha and TCR beta chains. The TCR complex is formed by TCR and CD3 gamma, CD3 delta, CD3 epsilon and CD3 zeta. The interruption or decreased expression of one or more of the chains T cell receptor alpha, T cell receptor beta, CD3 gamma, CD3 delta, CD3 epsilon or CD3 zeta can be used to reduce or prevent the formation of a functional T cell receptor (TCR) complex. By reducing or preventing the formation of a functional TCR complex, the T cell no longer mediates an immune response via its TCR complex. In an embodiment, a nucleic acid encoding a CAR is integrated at a site inside the cell's genome that interrupts or reduces the expression of a T cell receptor alpha chain, T cell receptor beta chain, CD3 gamma chain, CD3 delta chain, CD3 epsilon chain or CD3 zeta chain. While the reduction of one of the proteins of the TCR complex may be sufficient, it is understood that more than one component of the TCR complex can be reduced if desired.

Antigen-presenting cells activate the T cell receptor (TCR) and TCR-mediated signals are transmitted by the CD3 chains zeta, delta, epsilon, and gamma across the cell membrane. All CD3 chains contain immune receptor tyrosine-based activation motifs (ITAMs) in their cytoplasmic domain. CD3 zeta is part of the TCR-CD3 complex, which is present on the surface of T lymphocyte cells and plays an essential role in the adaptive immune response. CD3 zeta is encoded by the CD247 gene. Only endogenous CD3-zeta comprises three ITAMs and transmits an activation signal to the cell, e.g. a cell of the lymphatic line, such as a T-cell, after binding to the antigen. In some embodiments, a CAR comprises an intracellular CD3-zeta signal domain that can activate or stimulate a T-cell. The expression of the CD3 zeta/CD427 gene is the rate-limiting factor in the formation of the TCR complex and the CD247 expression is determining for the TCR complex assembly rate and function. It is understood that a CD3 zeta nucleic acid molecule refers to a polynucleotide encoding a CD3 zeta polypeptide.

Polypeptides

“Peptide” “polypeptide”, “polypeptide fragment” and “protein” are used interchangeably, unless specified to the contrary, and according to conventional meaning, i.e., as a sequence of amino acids. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring.

In various embodiments, the CAR polypeptides contemplated herein comprise a signal (or leader) sequence at the N-terminal end of the protein, which co-translationally or post-translationally directs transfer of the protein. Polypeptides can be prepared using any of a variety of well-known recombinant and/or synthetic techniques. Polypeptides contemplated herein specifically encompass the CARs of the present disclosure, or sequences that have deletions from, additions to, and/or substitutions of one or more amino acid of a CAR as disclosed herein.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. Similarly, an “isolated cell” refers to a cell that has been obtained from an in vivo tissue or organ and is substantially free of extracellular matrix.

Fusion Protein, Fusion Gene

“Fusion proteins” or “chimeric proteins” as used herein, comprise proteins formed by joining two or more nucleic acid segments that originally encoded separate proteins, polypeptides or protein fragments. “Fusion gene”, “gene fusion” or “fused gene” are according to the invention preferably joined nucleic acid sequence segments, wherein at least one nucleic acid sequence is also an exogenous nucleic acid sequence and at least one other nucleic acid sequence is also an endogenous nucleic acid sequence. In preferred embodiments, the exogenous nucleic acid is integrated into and fused to an endogenous nucleic acid sequence, e.g. an endogenous gene. In some embodiments, the integrated exogenous nucleic acid sequence to be fused may have several combined gene structures and/or polypeptide-encoding sequences separated by one or more protein separation sites.

The translation of a fusion gene results in a single or multiple polypeptides with functional properties derived from each of the original proteins or protein fragments. In a further embodiment, a fused gene may contain one or more nucleic acid sequences encoding a polypeptide, wherein the polypeptides may be present as individual polypeptides or proteins after translation, with separation being mediated by a protein separation site.

Chimera or chimeras refer to hybrid proteins made up of polypeptides with different functions or attributes. In some embodiments, fusion proteins combine whole peptides and therefore contain all functional domains of the original proteins. In certain embodiments, fusion proteins combine only parts of coding sequences and therefore only retain parts of the original functions of the genes from which they were formed.

A gene fusion can also cause regulatory changes that alter when, to what extent, and where these genes are expressed. In some embodiments, the rearrangement of different active sites and/or binding domains can additionally lead to the formation of proteins with new functions and/or specificities. A nucleic acid sequence encoding a CD3 zeta-deficient CAR fragment and a protein separation site may be fused within an endogenous gene, downstream of a promoter of an endogenous gene, within or at the junction site of an endogenous exon or within or at the junction site of an endogenous intron.

Nucleic Acids

As used herein, the terms “polynucleotide” or “nucleic acid molecule” refers to messenger RNA (mRNA), RNA, genomic RNA (gRNA), plus strand RNA (RNA(+)), minus strand RNA (RNA(−)), genomic DNA (gDNA), complementary DNA (cDNA) or recombinant DNA. Polynucleotides include single and double stranded polynucleotides. Preferably, polynucleotides of the invention include polynucleotides or variants having at least about 50%, 55%, 60%, 65%, 70%, 75%, 80%, 85%, 90%, 95%, 96%, 97%, 98%, 99% or 100%) sequence identity to any of the reference sequences described herein, typically where the variant maintains at least one biological activity of the reference sequence.

In various illustrative embodiments, the present invention contemplates, in part, polynucleotides comprising expression vectors, viral vectors, and transfer plasmids, and compositions, and cells comprising the same. Polynucleotides can be prepared, manipulated and/or expressed using any of a variety of well-established techniques known and available in the art. In order to express a desired polypeptide, a nucleotide sequence encoding the polypeptide, can be inserted into appropriate vector. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or MI 3 phage, and animal viruses. Examples of categories of animal viruses useful as vectors include, without limitation, retrovirus (including lentivirus), adenovirus, adeno-associated virus, herpesvirus {e.g., herpes simplex virus), poxvirus, baculovirus, papillomavirus, and papovavirus {e.g., SV40). Examples of expression vectors are pClneo vectors (Promega) for expression in mammalian cells; pLenti4V5-DEST™, pLenti6V5-DEST™, and pLenti6.2V5-GW/lacZ (Invitrogen) for lentivirus-mediated gene transfer and expression in mammalian cells. In particular embodiments, the coding sequences of the chimeric proteins disclosed herein can be ligated into such expression vectors for the expression of the chimeric protein in mammalian cells. The “control elements” or “regulatory sequences” present in an expression vector are those non-translated regions of the vector-origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

Vectors

In particular embodiments, a cell (e.g., an immune effector cell, such as a T cell) is transfected with a no-viral vector, a retroviral vector, e.g., a lentiviral vector, preferably a non-viral vector encoding a CAR. For example, an immune effector cell is transfected with a vector encoding a CAR that comprises a humanized anti-CD19 antibody or antigen binding fragment that binds a CD19 polypeptide, with a transmembrane and intracellular signaling domain, such that these transduced cells can elicit a CAR-mediated cytotoxic response.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In further embodiments of the invention, a gene editing technique, preferably CRISPR-Cas, mediated integration of the CD19 CAR encoding nucleic acid into a host genome may be employed. Appropriate vectors for CRISPR/Cas and TALEN-mediated insertion are known to a skilled person. In certain embodiments, the gene editing techniques include zinc finger nucleases (ZFNs), integrases, site specific recombinases, meganucleases, homing endonucleases, or TALENs, more preferably a CRISPR-Cas9 derived from Streptococcus pyogenes.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

In a preferred embodiment the invention therefore relates to a method for transfecting cells with an expression vector encoding a CAR. For example, in some embodiments, the vector comprises additional sequences, such as sequences that facilitate expression of the CAR, such a promoter, enhancer, poly-A signal, and/or one or more introns. In preferred embodiments, the CAR-coding sequence is flanked by transposon sequences, such that the presence of a transposase allows the coding sequence to integrate into the genome of the transfected cell.

In some embodiments, the genetically transformed cells are further transfected with a transposase that facilitates integration of a CAR coding sequence into the genome of the transfected cells. In some embodiments the transposase is provided as DNA expression vector. However, in preferred embodiments, the transposase is provided as an expressible RNA or a protein such that long-term expression of the transposase does not occur in the transgenic cells. For example, in some embodiments, the transposase is provided as an mRNA (e.g., an mRNA comprising a cap and poly-A tail). Any transposase system may be used in accordance with the embodiments of the present invention. However, in some embodiments, the transposase is salmonid-type Tel-like transposase (SB). For example, the transposase can be the so called “Sleeping beauty” transposase, see e.g., U.S. Pat. No. 6,489,458, incorporated herein by reference. In some embodiments, the transposase is an engineered enzyme with increased enzymatic activity. Some specific examples of transposases include, without limitation, SB 10, SB 11 or SB 100× transposase (see, e.g., Mates et al, 2009, Nat Genet. 41 (6):753-61, or U.S. Pat. No. 9,228,180). For example, a method can involve electroporation of cells with an mRNA encoding an SB 10, SB 11 or SB 100× transposase.

Gene Editing and Transfer

A gene editing technique can be used to interfere with the endogenous expression of any expressed structure on a genome, preferably the TCR complex component, CD5, CD7, CD52, MHC and/or a molecule involved in a control point gene. “Endogenous” as used herein is particularly used to define a gene expression that originates from within a system such as a host, organism, tissue, or cell whereas “exogenous” particularly defines a genetic sequence introduced from externally into a cell by one or more genetic, biochemical or other methods.

Gene editing is a type of genetic engineering in which DNA is inserted, deleted or replaced in the genome of a living organism by manipulated nucleases, or “molecular Scissors”. These nucleases generate site-specific double-strand breaks (DSBs) at desired sites in the genome. The induced double-strand breaks are repaired by non-homologous end compound (HEJ) or homologous recombination (HR), resulting in targeted mutations (“edits”). Five families of manipulated nucleases can be used for gene editing: (a) meganucleases, (b) zinc finger nuclei (ZFNs), (c) transcription activator-like effector-based nucleases (TALENs), (d) Mega-TALENs, and (e) the CRISPR-Cas system.

In certain embodiments, CRISPR-Cas9 is preferably used for gene editing. A “targeting sequence”, “target sequence” or “target site” mainly refers to a chromosomal or extrachromosomal nucleic acid sequence that defines a part of a nucleic acid to which a binding molecule binds and/or cleaves, provided there are sufficient conditions for binding and/or cleavage. In any of these gene editing approaches inducing double-strand breaks, the target sequence comprises an identical sequence to a recognition site of the endogenous DNA sequence into which an integration is intended, preferably selected from the group consisting of a homology arm, a guide RNA target site, a restriction enzyme recognition site, a ZFN recognition site, a ZFN cleaving site, a TALEN DNA-binding site, a recombinase recognition site, an integrase site and/or a homing nuclease recognition site Techniques using each of these artificial nucleases are well known among experts. For example, when cells are edited with a genome editing complex (e.g., a TALEN, CRISPR/Cas9, ZFN, Mega-TALENs, or meganucleases) to introduce a functional gene or to knock out a gene, nucleases (e.g., Fokl or Cas9) induce a double strand break at the site of modification. Upon induction of the double strand break, a protein, such as a DNA repair protein, e.g., phosphorylated (serl778) 53BP1 (p53BPI) or gH2AC can accumulate at the site of the double strand break and is indicative of a DNA damage response.

One or more of these techniques can be used alone or in any combination to disrupt endogenous expression of a TCR-complex component in the method of the invention. When the gene editing technique is used to disrupt the expression of a TCR complex component, the gene-editing technology can particularly target one or more of the following: (i) a CD3 zeta locus, (ii) a safe harbor locus, (iii) a TRAC locus; (ii) a locus controlling TCR-beta expression; (iii) a locus controlling TCR gamma expression; (iv) a locus that controls TCR delta expression; (v) a locus that controls the expression of a CD3 chain. CD3 chains are described above. The CD3 chain can be CD3 zeta, CD3 epsilon, CD3 delta or CD3 gamma. In certain embodiments, genome loci harboring genetic information for expressing at least one ITAM domain are preferably target for gene editing.

In some embodiments, a genetically modified cell or animal comprising an inactivated genomic sequence may be termed a “knock-out” or a “conditional knock-out.” Similarly, a genetically modified cell or animal comprising an integrated sequence may be termed a “knock-in” or a “conditional knock-in”.

Protein Separation Site

The term “protein separation site”, as used herein, comprise nucleic acid sequence elements that trigger separation of a bi-cistronic or a multi-cistronic expression of protein sequences in order to obtain distinctively expressed proteins. A protein separation site can, without limitation, refer to a self-cleavage peptide, an internal ribosome entry site (IRES), and/or a proteolytic cleavage site. In a preferred aspect of the invention, the nucleic acid construct can optionally be designed to include a protein separation site, preferably upstream of the nucleic acid sequences encoding a CAR or a CAR fragment. In particular embodiments, nucleic acid sequences considered herein, comprise one or more nucleic acid sequences regions of interest that encode one or more peptides and/or proteins. To achieve efficient translation of each of the numerous proteins, nucleic acid sequences regions can be separated by one or more IRES equivalents or nucleic acid sequences encoding for a self-cleaving peptide or a proteolytic cleavage site.

A “self-cleavage peptide” or “self-cleaving peptide”, e.g. P2A, can mediate ribosomal skipping during protein translation in a cell.

The term “internal ribosome entry site” or “IRES”, as used herein, refers to an element that promotes direct internal ribosome entry into the initiation codon, such as ATG, of a cistron (a protein coding region), resulting in RNA cap-independent translation of the gene. Examples of IRES generally used by professionals are those described in U.S. Pat. No. 6,692,736. Examples of “IRES” that are known to the scientific community include IRES that can be obtained from picornaviruses and IRES that can be obtained from viral or cellular mRNA sources, such as the immunoglobulin heavy chain binding protein (BiP), vascular endothelial growth factor (VEGF), fibroblast growth factor 2 (FGF-2) and insulin-like growth factor (IGFII), translational initiation factor elF4G and yeast transcription factors TFIID and HAP4, encephalomy-carditis virus (EMCV) commercially available from Novagen, and VEGF IRES IRES have also been detected in viral genomes of Picomaviridae, Dicistroviridae and Flaviviridae species as well as in HCV, Friend murine leukemia virus (FrMLV) and Moloney murine leukemia virus (MoMLV).

The term “proteolytic cleavage site” refers to the recognition nucleic acid sequence for an intracellular proteolytic cleavage enzyme breaking the peptide bonds between amino acids in proteins.

TCR Complex Component Promoter

The term “promoter” is used for a DNA sequence that initiates and regulates transcription from the DNA downstream of it through binding of proteins. The transcribed RNA may encode a protein. Promoters are preferably located near the transcription start sites of genes, upstream of the coding DNA (towards the 5′ region of the sense strand).

In some embodiments, it may be preferable to use a cell-type, cell lineage or tissue-specific expression control sequence to achieve cell-type, lineage or tissue-specific expression of a desired polynucleotide sequence (e.g. to express a particular nucleic acid encoding a peptide only in a subset of cell types, cell lines or tissues or during certain stages of development).

In aspects of the invention, a method is provided in which the regulation of an integrated transgene is placed under an endogenous promoter, whereby a nucleic acid construct of the transgene to be integrated is preferably promoter-deficient. In some embodiments, the nucleic acid construct of the transgene to be integrated encoding for a CAR or a CAR fragment, is preferably promoter-deficient. One such construct enables the integration of the transgene, encoding a CAR or a CAR fragment, into a location within the genome such that the integrated nucleic acid sequence, i.e. the transgene, is under the control of an endogenous promoter. In some embodiments, the endogenous promoter is a promoter of a TCR complex component. In some embodiments, the said endogenous promoter is preferably a promoter of genes encoding proteins of the TCR complex, e.g. a CD3 zeta, CD3 epsilon, CD3 delta, CD3 gamma, TCR alpha chain or TCR beta chain, preferably a gene encoding protein comprising at least one intracellular ITAM domain.

Expression Termination Signal

“Expression termination signal” comprises any structural elements at RNA and/or DNA level that specifies an end of transcription and/or translation, and/or a polyadenylation of the heterologous nucleic acid transcripts. The transcriptional stop is usually defined by a terminator, a nucleic acid sequence that defines the end of a transcription (e.g. of a gene) and initiates the release of the newly synthesized RNA. Terminators are located downstream of the gene to be transcribed and occur directly after 3′ regulatory elements, such as the poly-A signal. The term “poly-A site”, “poly-A signal” or “poly-A sequence”, as used herein, refers to a nucleic acid sequence that signals both, termination and polyadenylation, of the nascent RNA transcript by RNA polymerase II. Polyadenylation sequences can enhance mRNA stability by adding a poly-A tail at the 3′ end of the coding sequence, thus contributing to increased translation efficiency. Efficient polyadenylation of the recombinant transcript is preferable because transcripts without a poly-A tail are unstable and degrade rapidly. A “stop codon” is also a sequence of three consecutive nucleotides (termed triplet) within the messenger RNA that signals the termination of a protein translation process at the ribosome. In aspects of the invention, the CAR transgene preferably uses endogenous expression termination signals, including a poly-A signal, a stop codon for RNA translation into protein, and a transcriptional stop signal.

Integrated Sequence

An “integrated sequence” or “integrating sequence” particularly refers to a nucleic acid construct including coding and/or non-coding elements to be inserted into the genome of the transfected cell. In some embodiments, an edited chromosomal sequence may comprise an integrated sequence. The integrated sequence may code for an endogenous protein, an exogenous or heterologous protein, a wild-type protein, a modified protein, a fusion protein or similar. In some embodiments, the integrated sequence is also a transgene. In a specific aspect of the invention, different transgenes are integrated at the different integration sites. An “integration site” or “fusion site” as used herein is the exact nucleic acid position within a genome where a double-strand break is generated and a nucleic acid construct is incorporated into the genome by homology directed repair at the position of the double-strand break. An endogenous sequence can be interrupted by integrating an exogenous sequence, so that the exogenous sequence is under the control of the endogenous promoter. In some embodiments the integrated exogenous sequence and/or the integrated exogenous sequence would be expressed together with an endogenous sequence. An integrated protein coding sequence can also be placed under the control of an endogenous promoter and/or fused in-frame with an endogenous protein coding sequence. In addition, the integrated sequence can also serve as a control element. Accordingly, the integrated sequence may be endogenous or exogenous to the cell. The exogenous sequence may be a homologue of or related to an endogenous sequence. The exogenous sequence can also be a human sequence. An animal or cell with such an integrated sequence can be called a “knock-in”. In certain embodiments, a sequence can be integrated to change the expression pattern of a protein of interest. For example, a conditional knock-out system can be created. In aspects of the invention, an integrated sequence can be a nucleic acid sequence encoding a CAR, a CAR fragment, a protein separation site, a CAR regulatory protein and/or an immune stimulatory protein. In some embodiments, the nucleic acid construct integrated into the genome of the transfected cell comprise a CAR fragment and a protein separation site upstream of said CAR fragment.

“CD3 zeta KI” and “CD247 KI” and “zeta KI” refer to the CD247 gene comprising an exogenous nucleic sequence construct, e.g. the nucleic sequence construct described herein, integrated within the CD247 gene. In embodiments, the CAR fragment disclosed herein is integrated into CD3zeta/CD247 gene.

Nucleic Acids and Transfer Methods

Methods for introducing exogenous molecules, e.g. nucleic acid sequences, into cells are well known to experts and include lipid-mediated transfer (i.e. liposomes, including neutral and cationic lipids), electroporation, direct injection, cell fusion, particle bombardment, biopolymer nanoparticles, calcium phosphate co-precipitation, DEAE-dextran-mediated transfer and viral vector-mediated transfer. “Gene transfer”, “nucleic acid transfer”, gene sequence transfer”, “transgene transfer” are used interchangeably. In some embodiments, nucleic acid constructs are preferably transferred into cells by electroporation.

A “gene” refers to a DNA region that encodes a gene product including regions regulating the production of the gene product, regardless of whether these sequences are adjacent to coding and/or transcribed sequences. A gene comprises, but is not limited to, promoter sequences, terminators, translational regulatory sequences such as ribosome binding sites and internal ribosome entry sites, enhancers, silencers, insulators, boundary elements, replication origins, matrix attachment sites, and locus control regions.

“Gene expression” is particularly the conversion of the information encoded by a gene into a gene product. A gene product can be the direct transcription product of a gene (e.g., mRNA, tRNA, rRNA, antisense RNA, ribozyme, structural RNA or any other type of RNA) or a protein produced by translation of a mRNA. Gene products also include RNAs modified by processes such as capping, polyadenylation, methylation, and editing, as well as proteins modified by processes such as methylation, acetylation, phosphorylation, ubiquitination, ADP-ribosylation, myristoylation, and glycosylation.

Sequence Variation:

Sequence variants of the claimed nucleic acids, proteins, antibodies, antibody fragments and/or CARs, for example those defined by % sequence identity, that maintain similar binding properties of the invention are also included in the scope of the invention. Such variants, which show alternative sequences, but maintain essentially the same binding properties, such as target specificity, as the specific sequences provided are known as functional analogues, or as functionally analogous.

Sequence identity relates to the percentage of identical nucleotides or amino acids when carrying out a sequence alignment. The recitation “sequence identity” as used herein refers to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

It will be appreciated by those of ordinary skill in the art that, as a result of the degeneracy of the genetic code, there are many nucleotide sequences that encode a polypeptide as described herein. Some of these polynucleotides bear minimal homology or sequence identity to the nucleotide sequence of any native gene. Nonetheless, polynucleotides that vary due to differences in codon usage are specifically contemplated by the present invention. Deletions, substitutions and other changes in sequence that fall under the described sequence identity are also encompassed in the invention.

Protein sequence modifications, which may occur through substitutions, are also included within the scope of the invention. Substitutions as defined herein are modifications made to the amino acid sequence of the protein, whereby one or more amino acids are replaced with the same number of (different) amino acids, producing a protein which contains a different amino acid sequence than the primary protein. Substitutions may be carried out that preferably do not significantly alter the function of the protein. Like additions, substitutions may be natural or artificial. It is well known in the art that amino acid substitutions may be made without significantly altering the protein's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the protein. Frequently, many amino acids may be substituted by conservative amino acids without deleteriously affecting the protein's function.

In general, the non-polar amino acids Gly, Ala, Val, lie and Leu; the non-polar aromatic amino acids Phe, Trp and Tyr; the neutral polar amino acids Ser, Thr, Cys, Gin, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups.

Substitution variants have at least one amino acid residue in the antibody molecule removed and a different residue inserted in its place. The sites of greatest interest for substitutional mutagenesis include the hypervariable regions, but framework alterations are also contemplated. If such substitutions result in a change in biological activity, then more substantial changes, denominated “exemplary substitutions” in the table immediately below, or as further described below in reference to amino acid classes, may be introduced and the products screened.

Potential Amino Acid Substitutions:

| | Preferred | |
|---|---|---|
| Original | conservative | |
| residue | substitutions | Examples of exemplary substitutions |
| Ala (A) | Val | Val; Leu; Ile |
| Asg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn, Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Conservative amino acid substitutions are not limited to naturally occurring amino acids, but also include synthetic amino acids. Commonly used synthetic amino acids are omega amino acids of various chain lengths and cyclohexyl alanine which are neutral non-polar analogues; citrulline and methionine sulfoxide which are neutral non-polar analogues, phenylglycine which is an aromatic neutral analog; cysteic acid which is a negatively charged analog and ornithine which is a positively charged amino acid analogue. Like the naturally occurring amino acids, this list is not exhaustive, but merely exemplary of the substitutions that are well known in the art.

Genetically Modified Cells and Immune Cells

The present invention contemplates, in particular embodiments, cells genetically modified to express the CARs contemplated herein, for use in the treatment of medical conditions. As used herein, the term “genetically engineered” or “genetically modified” refers to the addition of extra genetic material in the form of DNA or RNA into the total genetic material in a cell. Genetic modifications can be carried out selectively or not at a specific location in the genome of a cell. In an embodiment, the genetic change is site-specific. In an embodiment, the genetic modification is not site-specific. The terms, “genetically modified cells”, “genetically modified immune cells”, “modified cells,” and, “redirected cells,” are used interchangeably. As used herein, the term “gene therapy” refers to the introduction-permanently or transiently of extra genetic material in the form of DNA or RNA into the total genetic material in a cell that restores, corrects, or modifies expression of a gene, or for the purpose of expressing a therapeutic polypeptide, e.g., a CAR. In particular embodiments, the CARs contemplated herein are introduced and expressed in immune effector cells so as to redirect their specificity to a target antigen of interest, e.g., a CD19 polypeptide.

An “immune cell” or “immune effector cell” is any cell of the immune system that has one or more effector functions (e.g., cytotoxic cell killing activity, secretion of cytokines, induction of ADCC and/or CDC). An immune effector cell can be also differentiated from iPSCs (induced pluripotent stem cells) or derived from peripheral human blood and/or human cord blood.

Immune effector cells of the invention can be autologous/autogeneic (“self”) or non-autologous (“non-self,” e.g., allogeneic, syngeneic or xenogeneic). “Autologous”, as used herein, refers to cells from the same subject, and represent a preferred embodiment of the invention. “Allogeneic,” as used herein, refers to cells of the same species that differ genetically to the cell in comparison.

“Syngeneic,” as used herein, refers to cells of a different subject that are genetically identical to the cell in comparison. “Xenogeneic,” as used herein, refers to cells of a different species to the cell in comparison. In preferred embodiments, the cells of the invention are autologous or allogeneic. Illustrative immune effector cells used with the CARs contemplated herein include T lymphocytes. The terms “T cell” or “T lymphocyte” are art-recognized and are intended to include thymocytes, immature T lymphocytes, mature T lymphocytes, resting T lymphocytes, cytokine-induced killer cells (CIK cells) or activated T lymphocytes. Cytokine-induced killer (CIK) cells are typically CD3- and CD56-positive, non-major histocompatibility complex (MHC)—restricted, natural killer (NK)-like T lymphocytes. A T cell can be a T helper (Th; CD4+ T cell) cell, for example a T helper (Th) cell, such as a TH1, TH2, TH3, TH17, TH9 or TFH cell. The T cell can be a cytotoxic T cell (CTL; CD8+ T cell), CD4+CD8+ T cell, CD4 CD8 T cell, or a regulatory T cell (reg) or any other subset of T cells. The T cell can be a naive, effector, memory, effector storage, central storage, memory stem T cell. The T cell can be an umbilical cord blood cell. The T cell can be a peripheral lymphocyte.

The T-cell may be expanded from PBMNCs. The T-cell may be autologous with respect to an individual to whom it is to be administered. The T-cell may be allogeneic with respect to an individual to whom it is to be administered. The T-cell may be partially HLA incompatible with an individual to whom it is to be administered.

“Immortalized”, as used herein, refers to immortalized cells as a population of cells that would not normally reproduce for an unlimited number of cell cycles. Due to a mutation, immortal cells escape from a normal cellular senescence and instead keep undergoing the cell proliferation. The mutation can occur spontaneously or induced by UV light, genetic manipulation, or entry by a virus, toxin or bacteria into the cell. The cells can therefore be cultivated in vitro over a longer period of time for experimental, therapeutic or medical purposes. Said immortalized immune cells include K13, αβ T cells, γδ T cells, NK cells, NKT cells, NK-92 and YT cells, stem cells, or stem cell-derived cells including cells of the aforementioned immune system, preferably NKT cells, NK-92 cells, YT cells, and NK cells. An immortalized T cell line may retains its lytic function.

Other illustrative populations of T cells suitable for use in particular embodiments include naive T cells and memory T cells and stem cell-like memory cells TSCM).

For example, when reintroduced back to patients after autologous cell transplantation, the T cells modified with the CAR of the invention as described herein may recognize and kill tumor cells. CIK cells may have enhanced cytotoxic activity compared to other T cells, and therefore represent a preferred embodiment of an immune cell of the present invention.

As would be understood by the skilled person, other cells may also be used as immune effector cells with the CARs as described herein. In particular, immune effector cells also include NK cells, NKT cells, neutrophils, and macrophages. Immune effector cells also include progenitors of effector cells wherein such progenitor cells can be induced to differentiate into an immune effector cells in vivo or in vitro. Progenitors can be iPSCs that become immune effector cells under defined culture conditions. The present invention provides methods for making the immune effector cells which express the CAR contemplated herein. In one embodiment, the method comprises transfecting or transducing immune effector cells isolated from an individual such that the immune effector cells express one or more CAR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAR. In this regard, the immune effector cells may be cultured before and/or after being genetically modified (i.e., transduced or transfected to express a CAR contemplated herein).

In particular embodiments, prior to in vitro manipulation or genetic modification of the immune effector cells described herein, the source of cells is obtained from a subject. In particular embodiments, the CAR-modified immune effector cells comprise T cells. T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation, antibody-conjugated bead-based methods such as MACS™ separation (Miltenyi). In one embodiment, cells from the circulating blood of an individual are obtained by apheresis. The apheresis product typically contains lymphocytes, including T cells, monocytes, granulocyte, B cells, other nucleated white blood cells, red blood cells, and platelets. In one embodiment, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. As would be appreciated by those of ordinary skill in the art, a washing step may be accomplished by methods known to those in the art, such as by using a semiautomated flow through centrifuge. For example, the Cobe 2991 cell processor, the Baxter CytoMate, or the like. After washing, the cells may be resuspended in a variety of biocompatible buffers or other saline solution with or without buffer. In certain embodiments, the undesirable components of the apheresis sample may be removed in the cell directly resuspended culture media.

In certain embodiments, T cells are isolated from peripheral blood mononuclear cells (PBMCs) by lysing the red blood cells and depleting the monocytes, for example, by centrifugation through a PERCOLL™ gradient. A specific subpopulation of T cells can be further isolated by positive or negative selection techniques. One method for use herein is cell sorting and/or selection via negative magnetic immunoadherence or flow cytometry that uses a cocktail of monoclonal antibodies directed to cell surface markers present on the cells negatively selected.

PBMC may be directly genetically modified to express CARs using methods contemplated herein. In certain embodiments, after isolation of PBMC, T lymphocytes are further isolated and in certain embodiments, both cytotoxic and helper T lymphocytes can be sorted into naive, memory, and effector T cell subpopulations either before or after genetic modification and/or expansion. CD8+ cells can be obtained by using standard methods. In some embodiments, CD8+ cells are further sorted into naive, central memory, and effector cells by identifying cell surface antigens that are associated with each of those types of CD8+ cells.

In some embodiments, the immune cell of the present invention, for example the T cells described herein, can be obtained from inducible pluripotent stem cells (iPSCs) using methods known to a skilled person. Accepted approaches for producing CAR-T cells rely on the genetic modification and expansion of mature circulating T cells. Such processes utilize autologous T cells and reduce risk of graft-versus-host (GvHD) disease from allogeneic T cells through endogenous TCR expression as well as rejection through MHC incompatibility. As an alternative, direct in vitro differentiation of engineered T cells from pluripotent stem cells, such as inducible pluripotent stem cells, provides an essentially unlimited source of cells that can be genetically modified to express the CAR of the present invention. In some embodiments, a so-called master iPSC line can be maintained, which represents a renewable source for consistently and repeatedly manufacturing homogeneous cell products. In some embodiments, the transformation of a master iPSC cell line with the CAR encoding nucleic acid is contemplated, prior to expansion and differentiation to the desired immune cell, preferably T cell or NK cell. T lymphocytes can for example be generated from iPSCs, such that iPSCs could be modified with the CAR or the CD3 zeta-deficient CAR fragment encoding nucleic acids and subsequently expanded and differentiated to T cells for administration to the patient. Differentiation to the appropriate immune cell, such a T cell, could also be conducted from the iPSCs before transformation with the CAR or the CD3 zeta-deficient CAR encoding nucleic acids and expansion prior to administration. All possible combinations of iPSC expansion, genetic modification and expansion to provide suitable numbers of cells for administration are contemplated in the invention.

The immune effector cells, such as T cells, NK cells, CIK cells or Tregs, can be genetically modified following isolation using known methods, or the immune effector cells can be activated and expanded (or differentiated in the case of progenitors) in vitro prior to being genetically modified. In a particular embodiment, the immune effector cells, such as T cells, NK cells, CIK cells or Tregs, are genetically modified with the CD3 zeta-deficient CAR fragment contemplated herein (e.g., transfected with a nucleic acid and pre-complexed Cas9 enzyme or transduced with a viral vector comprising a nucleic acid encoding a CD3 zeta-deficient CAR fragment) and then are activated and expanded in vitro. In various embodiments, T cells can be activated and expanded before or after genetic modification to express a CAR or a CD3 zeta-deficient CAR fragment, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In a further embodiment, a mixture of, e.g., one, two, three, four, five or more, different expression vectors can be used in genetically modifying a donor population of immune effector cells wherein each vector encodes a different chimeric antigen receptor protein as contemplated herein. The resulting modified immune effector cells forms a mixed population of modified cells, with a proportion of the modified cells expressing more than one different CAR protein.

In one embodiment, the invention provides a method of storing genetically modified murine, human or humanized CAR protein expressing immune effector cells which target a CD19 protein, comprising cryopreserving the immune effector cells such that the cells remain viable upon thawing. A fraction of the immune effector cells expressing the CAR proteins can be cryopreserved by methods known in the art to provide a permanent source of such cells for the future treatment of patients afflicted with the B cell, T cell, NK cell, dendritic cell (DC), cytotoxic induced killer cell (CIK) related condition. When needed, the cryopreserved transformed immune effector cells can be thawed, grown and expanded for more such cells.

Upstream/Downstream

“Upstream” and “downstream” as used herein, are employed in the context of the 5′→3′ direction of a nucleic acid strand. Upstream refers to nucleotides towards the 5′ end and downstream towards the 3′ end of any given nucleic acid.

In-Frame, Out-of-Frame

A fusion product is defined as ‘in-frame’ when an open reading frame remains intact in the 3′-region downstream of the nucleic acid integration site, regardless of the number of inserted or deleted amino acids at the fusion junction point. A shift in the open reading frame of a protein coding gene sequence refers to “out-of-frame”.

Compositions and Formulations

The compositions contemplated herein may comprise one or more polypeptides, polynucleotides, vectors comprising same, genetically modified immune effector cells, etc., as contemplated herein. Compositions include, but are not limited to, pharmaceutical compositions.

A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions of the invention may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals.

Plasmids, nucleic acids, nucleic acids complexed with a Cas enzyme, AAV vectors, lentiviral vectors and/or other compositions, agents, drugs, biologics (proteins) can be incorporated into pharmaceutical compositions, e.g., pharmaceutically acceptable carriers or excipients. Such pharmaceutical compositions are particularly useful for administration and delivery to a subject in vivo or ex vivo.

In particular embodiments, compositions of the present invention comprise an amount of CAR-expressing immune effector cells contemplated herein. As used herein, the term “amount” refers to “an amount effective” or “an effective amount” of a genetically modified therapeutic cell, e.g., T cell, NK cell, CIK cell, or Treg cell, to achieve a beneficial or desired prophylactic or therapeutic result, including clinical results.

A “prophylactically effective amount” refers to an amount of a genetically modified therapeutic cell effective to achieve the desired prophylactic result. Typically but not necessarily, since a prophylactic dose is used in subjects prior to or at an earlier stage of disease, the prophylactically effective amount is less than the therapeutically effective amount. The term prophylactic does not necessarily refer to a complete prohibition or prevention of a particular medical disorder. The tern prophylactic also refers to the reduction of risk of a certain medical disorder occurring or worsening in its symptoms.

A “therapeutically effective amount” of a genetically modified immune cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced or transfected therapeutic cells are outweighed by the therapeutically beneficial effects. The term “therapeutically effective amount” includes an amount that is effective to “treat” a subject {e.g., a patient). When a therapeutic amount is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells or CIK cells or NK cells or Treg cells described herein may be administered at a dosage of 10² to 10¹⁰ cells/kg body weight, preferably 10⁵ to 10⁷ cells/kg body weight, including all integer values within those ranges. The number of cells will depend upon the ultimate use for which the composition is intended as will the type of cells included therein. For uses provided herein, the cells are generally in a volume of a liter or less, can be 500 mL or less, even 250 mL or 100 mL or less. Hence the density of the desired cells is typically greater than 10⁶ cells/ml and generally is greater than 10⁷ cells/mL, generally 10⁸ cells/mL or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present invention, particularly since all the infused cells will be redirected to a particular target antigen, lower numbers of cells may be administered. CAR expressing cell compositions may be administered multiple times at dosages within these ranges. The cells may be allogeneic, syngeneic, xenogeneic, or autologous to the patient undergoing therapy.

Generally, compositions comprising the cells activated and expanded as described herein may be utilized in the treatment and prevention of diseases that arise in individuals who are immunocompromised. In particular, compositions comprising the CAR-modified T cells, CAR-modified NK cells, CAR-modified CIK cells or CAR-modified Treg cells contemplated herein are used in the treatment of cancer, preferably solid or liquid, more preferably solid malignancies, more preferably rectal cancer, lung cancer, breast cancer, liver cancer, pancreatic cancer, stomach cancer, and ovarian cancer, more preferably metastatic tumor cells positive for CD19. The CAR-modified T cells, CAR-modified NK cells, CAR-modified CIK cells or CAR-modified Treg cells of the present invention may be administered either alone, or as a pharmaceutical composition in combination with carriers, diluents, excipients, and/or with other components such as interleukins or other immune response stimulating cytokines e.g. IL-15 and/or checkpoint inhibitory molecules, e.g. PD1 polypeptide or a PD1-antibody, or cell populations.

In particular embodiments, pharmaceutical compositions contemplated herein comprise an amount of genetically modified T cells, in combination with one or more pharmaceutically or physiologically acceptable carriers, diluents or excipients. In particular embodiments, pharmaceutical compositions contemplated herein comprise an amount of genetically modified NK cells, genetically modified CIK cells or genetically modified Treg cells in combination with one or more pharmaceutically or physiologically acceptable carriers, diluents or excipients.

Pharmaceutical compositions of the present invention comprising a CAR-expressing immune effector cell population, such as T cells or NK cells or CIK cells or Treg cells, may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for parenteral administration, e.g., intravascular (intravenous or intraarterial), intraperitoneal or intramuscular administration.

The liquid pharmaceutical compositions, whether they are solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In a particular embodiment, compositions contemplated herein comprise an effective amount of CAR-expressing immune effector cells, alone or in combination with one or more therapeutic agents. Thus, the CAR-expressing immune effector cell compositions may be administered alone or in combination with other known cancer treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated include cytokines, growth factors, steroids, NSAIDs, DMARDs, anti-inflammatories, chemotherapeutics, radiotherapeutics, therapeutic antibodies, or other active and ancillary agents.

A combined immunotherapy encompasses simultaneous treatment, co-treatment orjoint treatment, and includes the administration of genetically modified immune cells expressing a nucleic acid construct encoding a CAR or a CD3 zeta-deficient CAR fragment combined with immunotherapies, such as checkpoint inhibitors and/or immune stimulatory cytokines, whereby treatment may occur within minutes of each other, in the same hour, on the same day, in the same week or in the same month as one another. A combination medicament, comprising one or more of said genetically modified immune cells with another immunotherapeutic, may also be used in order to co-administer the various components in a single administration or dosage. A “medicament” particularly refers to a drug including an immune-therapeutic cell or genetically modified cell used to cure, treat, or prevent a disease.

As used herein, the term “tumor microenvironment” relates to the cellular environment in which any given tumor exists, including the tumor stroma, surrounding blood vessels, immune cells, fibroblasts, other cells, signaling molecules, and the ECM.

Therapeutic Methods

The genetically modified immune effector cells contemplated herein provide improved methods of adoptive immunotherapy for use in the treatment of medical disorders associated with the presence of pathogenic cells expressing CD19 that include, but are not limited to, immunoregulatory conditions and/or hematological and/or solid malignancies.

As used herein, “medical disorders associated with the presence of pathogenic cells expressing CD19 refer to medical conditions, such as a cancer or an autoimmune disease, in which the cells involved in pathophysiology of the disease demonstrate expression of CD19, and preferably presentation of CD19 on the cell surface. The expression of CD19 can be determined by various methods known to a skilled person, for example by isolating cells from a patient and assessing these by PCR using primers directed CD19 transcripts, immuno-staining with anti CD19 antibodies, or by analysis by flow cytometry. Such pathogenic cells may typically be pathogenic mature B cells and/or memory B cells and/or pathogenic T cells and/or T follicular helper cells and/or tumor stem cells and/or solid tumor cells and/or liquid tumors and/or metastatic cancer cells and/or NK cells and/or CIK cells, and/or Treg cells, and/or dendritic cells.

“Cancer”, as used herein, is a disease characterized by the uncontrolled growth of abnormal cells. Cancer refers to any type of cancerous growth or carcinogenic process, metastatic tissue or malignant transformed cells, tissues or organs, regardless of histopathological type or invasive stage. Cancer cells can spread locally or to other parts of the body via the bloodstream and lymphatic system. Cancer cells spreading to other parts to the body are termed “metastatic cells” or “metastatic tumor cells”. The terms “tumor” and “cancer”, used herein, are utilized interchangeably, e.g. both terms include solid and liquid, e.g. general or circulating tumors, premalignant and malignant cancers and tumors. Examples of liquid cancers include, but not limited to, acute lymphocytic leukemia (ALL), chronic lymphocytic leukemia (CLL), acute myeloid Leukemia (acute myelogenous leukemia (AML), chronic bone marrow cancer, chronic myelogenous leukemia (CML), Hodgkin lymphoma, non-Hodgkin lymphoma, and myeloma. Examples of solid tumors are liver, lung, breast, lymphatic system, digestive organs (e.g. colon), urogenital organs (e.g. kidney, urothelial cells), prostate and throat, malignant organ systems including tumors such as sarcomas, adenocarcinomas and cancer. Examples for breast cancer that can be treated with the are ductal carcinoma in situ (DCIS), lobular carcinoma in situ (LCIS), invasive ductal carcinoma (IDC), invasive ductal carcinoma including tubular, medullary, mucinous, papillary, and cribriform carcinomas, invasive lobular carcinoma (ILC), inflammatory breast cancer, male breast cancer, Paget's Disease of the nipple, phyllodes tumors of the breast, recurrent and/or metastatic breast cancer. Adenocarcinoma includes most malignant tumors such as colon cancer, rectal cancer, renal cell cancer, liver cancer, non-small cell lung cancer, small intestine cancer and esophageal cancer. In certain forms the cancer is a melanoma, e.g. an advanced stage melanoma. Metastatic lesions of the cancer can also be treated or prevented with the methods and compositions of the invention. Examples of other types of cancer that can be treated are bone cancer, pancreatic cancer, skin cancer, head and neck cancer, skin or intraocular melanoma, uterine cancer, ovarian cancer, rectal cancer, anal cancer, stomach cancer, testicular cancer, faropius duct cancer, Endometrial cancer, cervical cancer, vaginal cancer, vulvar cancer, Hodgkin's disease, non-Hodgkin's lymphoma, esophageal cancer, small intestine cancer, endocrine cancer, thyroid cancer, parathyroid cancer, adrenal cancer, soft tissue sarcoma, urethral cancer, penile cancer, acute myeloid leukemia, chronic myelocytic leukemia, acute lymphoblastic leukemia, chronic or acute leukemia including chronic lymphatic leukemia, solid tumor in childhood, lymphatic lymphoma, bladder cancer, kidney or ureter cancer, renal pelvis cancer, neoplasm of the central nervous system (CNS) primary CNS lymphoma, tumor angiogenesis, spinal axis tumor, brainstem glioma, pituitary adenoma, Kaposi's sarcoma, epidermoid carcinoma, squamous cell carcinoma, asbestos-induced T cell lymphoma Including a combination of environmental cancer and cancer including things Treatment of metastatic cancer, e.g. metastatic cancer that expresses PD-L1 (lwai et al. (2005) Int. Immunol. 17: 133-144) can be performed with the inhibitory molecules described in this invention.

“Cancer-associated antigen” or “tumor antigen” is expressed interchangeably on the surface of cancer cells, either completely or as a fragment (e.g. MHC/peptide), and the drug targets cancer cells preferentially. Molecules (usually proteins, carbohydrates or lipids) that are useful for. Tumor antigen refers to an antigen that is commonly found in a specific hyperproliferative disease. In one aspect, the antigen of hyperproliferative disease of the present invention is a primary or metastatic melanoma, thymoma, lymphoma, sarcoma, lung cancer, liver cancer, non-Hodgkin's lymphoma, Hodgkin's lymphoma, Leukemia, uterine cancer, cervical cancer, bladder cancer, derived from cancers such as kidney and breast cancer, prostate cancer, ovarian cancer, adenocarcinoma such as pancreatic cancer and the like. In certain embodiments, the tumor antigen is a marker expressed on both normal and cancer cells, such as a marker of cell lineage, like CD19 on B cells. In certain embodiments, the tumor antigen is overexpressed in cancer cells compared to normal cells, e.g. 1-fold overexpression, 2-fold overexpression, 3-fold overexpression or more overexpression compared to normal cells. Molecules of the cell surface. In certain forms, a tumor antigen is a cell surface molecule that is inadequately synthesised in cancer cells, e.g. a molecule that contains deletions, additions or mutations compared to a molecule expressed in normal cells. In certain embodiments, the tumor antigen is expressed exclusively, completely or as a fragment (e.g. MHC/peptide) on the cell surface of cancer cells and is not synthesised or expressed on the surface of normal cells. In certain embodiments, a CAR of the invention comprises a CAR containing an antigen binding domain (e.g. an antibody or antibody fragment) which binds to an MHC-presenting peptide. Normally, peptides derived from endogenous proteins fill the pockets of major histocompatibility complex (MHC) class I molecules and are recognized by the T cell receptor (TCR) on CD8+T lymphocytes. Class I MHC complexes are constitutively expressed by all nucleated cells. In cancer, virus- and/or tumor-specific peptides/MHC complexes represent a unique class of cell surface targets for immunotherapy. TCR-like antibodies have been described which target peptides of virus or tumor antigens associated with human leukocyte antigen (HLA) —Al or HLA-A2 (see e.g. Sastry et al., J Virol. 2011 85 (5): 1935-1942; Sergeeva et al, Blood, 2011 117 (16): 4262-4272; Verma et al., J Immunol 2010 184 (4): 2156-2165; Willemsen et al., Gene Ther 2001 8 (21): 1601-1608; Dao et al., Sci Transl Med 2013 5 (176): 176ra33; Tassev et al., Cancer Gene Ther 2012 19 (2): 84-100). For example, TCR-like antibodies can be identified by searching a library such as a human scFv phage display library.

In particular embodiments, compositions comprising CAR-modified T cells contemplated herein are used in the treatment of cancer, including but not limited to solid or liquid/hematological malignancies, such as, for example, rectal cancer, lung cancer, breast cancer, liver cancer, pancreatic cancer, stomach cancer, ovarian cancer, and metastatic tumor cells positive for CD19, or hematological malignancies such as, leukemia and lymphoma or myeoloproliferative disorders/neoplasms with or without a leukemic tumor cell dissemination, preferably solid tumors and multiple myeloma.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” or “prophylactic” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

In one embodiment, a method of treating a cancer related condition in a subject in need thereof comprises administering an effective amount, e.g., therapeutically effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

Sequences

Preferred Amino Acid Sequences of the Invention:

| | Homology- | | |
|---|---|---|---|
| Seq_ | Directed DNA | Info | |
| ID | Repair (HDR) | HDR-Template | |
| NO | Template | Part | Amino acid sequence |
| 1 | CD247- | Full Sequence | aqsfglldpklcylldgilfiyrviltalflrATNFSLLKQAGDVEENPG |
| | exon2_ | | PMALPVTALLLPLALLLHAARPDIQMTQTTSSLSASLGDRVTISCRASQ |
| | CD19CAR | | DISKYLNWYQQKPDGTVKLLIYHTSRLHSGVPSRFSGSGSGTDYSLTISN |
| | (truncated) | | LEQEDIATYFCQQGNTLPYTFGGGTKLEITGGGGGGGGSGGGGSEV |
| | | | KLQESGPGLVAPSQSLSVTCTVSGVSLPDYGVSWIRQPPRKGLEWLGV |
| | | | IWGSETTYYNSALKSRLTIIKDNSKSQVFLKMNSLQTDDTAIYYCAKHY |
| | | | YYGGSYAMDYWGQGTSVTVSSESKYGPPCPPCPGQPREPQVYTLPPSRD |
| | | | ELTKNQVSLTCLVKGFYPSDIAVEWESNGQPENNYKTTPPVLDSDGSF |
| | | | FLYSKLTVDKSRWQQGNVFSCSVMHEALHNAYTQKSLSLSPGKKDP |
| | | | KFWVLVVVGGVLACYSLLVTVAFIIFWVRSKRSRLLHSDYMNMTPRRP |
| | | | GPTRKHYQPYAPPRDFAAYRSrvk |
| 2 | | Left Homology | aqsfglldpklcylldgilfiyrviltalflr |
| | | Arm | |
| 3 | | P2A | ATNFSLLKQAGDVEENPGP |
| 4 | | Membrane | MALPVTALLLPLALLLHAARP |
| | | Export Signal | |
| | | (Leader) | |
| 5 | | FMC63VL (Light | DIQMTQTTSSLSASLGDRVTISCRASQDISKYLNWYQQKPDGTVKLLIYH |
| | | Chain, Variable | TSRLHSGVPSRFSGSGSGTDYSLTISNLEQEDIATYFCQQGNTLPYTFG |
| | | Part) | GGTKLEIT |
| 6 | | (G4S)3 Linker | GGGGSGGGGSGGGGS |
| 7 | | FMC63VH | EVKLQESGPGLVAPSQSLSVTCTVSGVSLPDYGVSWIRQPPRKGLEWL |
| | | (Heavy Chain, | GVIWGSETTYYNSALKSRLTIIKDNSKSQVFLKMNSLQTDDTAIYYCAKH |
| | | Variable Part) | YYYGGSYAMDYWGQGTSVTVSS |
| 8 | | IgG4 Hinge | ESKYGPPCPPCP |
| 9 | | IgG1 CH3 | GQPREPQVYTLPPSRDELTKNQVSLTCLVKGFYPSDIAVEWESNGQPE |
| | | Domain (Hinge) | NNYKTTPPVLDSDGSFFLYSKLTVDKSRWQQGNVFSCSVMHEALHNAY |
| | | | TQKSLSLSPGK |
| 10 | | Linker | KDPK |
| 11 | | CD28 | FWVLVVVGGVLACYSLLVTVAFIIFWV |
| | | Transmembrane | |
| | | Domain | |
| 12 | | CD28 | RSKRSRLLHSDYMNMTPRRPGPTRKHYQPYAPPRDFAAYRS |
| | | Intracellular | |
| | | Domain | |
| 13 | | Right Homology | rvk |
| | | Arm | |
| 14 | CD247- | Full Sequence | aqsfglldpklcylldgilfiyrviltalflrATNFSLLKQAGDVEEN |
| | exon2_ | | PGPMALPVTALLLPLALLLHAARPDIQMTQTTSSLSASLGDRVTISCRA |
| | CD19CAR | | SQDISKYLNWYQQKPDGTVKLLIYHTSRLHSGVPSRFSGSGSGTDYSLTI |
| | (full) | | SNLEQEDIATYFCQQGNTLPYTFGGGTKLEITGGGGSGGGGSGGGGS |
| | | | EVKLQESGPGLVAPSQSLSVTCTVSGVSLPDYGVSWIRQPPRKGLEWL |
| | | | GVIWGSETTYYNSALKSRLTIIKDNSKSQVFLKMNSLQTDDTAIYYCAK |
| | | | HYYYGGSYAMDYWGQGTSVTVSSESKYGPPCPPCPGQPREPQVYTLPP |
| | | | SRDELTKNQVSLTCLVKGFYPSDIAVEWESNGQPENNYKTTPPVLDSD |
| | | | GSFFLYSKLTVDKSRWQQGNVFSCSVMHEALHNAYTQKSLSLSPGKK |
| | | | DPKFWVLVVVGGVLACYSLLVTVAFIIFWVRSKRSRLLHSDYMNMTP |
| | | | RRPGPTRKHYQPYAPPRDFAAYRSRVKFSRSADAPAYQQGQNQLYNE |
| | | | LNLGRREEYDVLDKRRGRDPEMGGKPRRKNPQEGLYNELQKDKMAE |
| | | | AYSEIGMKGERRRGKGHDGLYQGLSTATKDTYDALHMQALPPR* |
| 15 | CD247- | Full Sequence | fsrsadapayATNFSLLKQAGDVEENPGPMALPVTALLLPLALLLHAARPDIQ |
| | exon3_ | | MTQTTSSLSASLGDRVTISCRASQDISKYLNWYQQKPDGTVKLLIYHTS |
| | CD19CAR | | RLHSGVPSRFSGSGSGTDYSLTISNLEQEDIATYFCQQGNTLPYTFGGG |
| | (truncated) | | TKLEITGGGGSGGGGSGGGGSEVKLQESGPGLVAPSQSLSVTCTVSG |
| | | | VSLPDYGVSWIRQPPRKGLEWLGVIWGSETTYYNSALKSRLTIIKDNSKS |
| | | | QVFLKMNSLQTDDTAIYYCAKHYYYGGSYAMDYWGQGTSVTVSSESKY |
| | | | GPPCPPCPGQPREPQVYTLPPSRDELTKNQVSLTCLVKGFYPSDIAVE |
| | | | WESNGQPENNYKTTPPVLDSDGSFFLYSKLTVDKSRWQQGNVFSCSV |
| | | | MHEALHNAYTQKSLSLSPGKKDPKFWVLVVVGGVLACYSLLVTVAFIIF |
| | | | WVRSKRSRLLHSDYMNMTPRRPGPTRKHYQPYAPPRDFAAYRSRVKF |
| | | | SRSAdapayqqgqnqlyn |
| 16 | | Left Homology | fsrsadapay |
| | | Arm | |
| 17 | | CAR body | SEQ ID NO 3-SEQ ID 12 |
| 18 | | truncated CD3- | RVKFSRSA |
| | | zeta | |
| 19 | | Right Homology | dapayqqgqnqlyn |
| | | Arm with Silent | |
| | | Mutations to | |
| | | Prevent Cas9 | |
| | | Re-Cutting | |
| 20 | CD247- | Full Sequence | fsrsadapayATNFSLLKQAGDVEENPGPMALPVTALLLPLALLLHAARPDIQ |
| | exon3_ | | MTQTTSSLSASLGDRVTISCRASQDISKYLNWYQQKPDGTVKLLIYHTS |
| | CD19CAR | | RLHSGVPSRFSGSGSGTDYSLTISNLEQEDIATYFCQQGNTLPYTFGGG |
| | (full) | | TKLEITGGGGSGGGGSGGGGSEVKLQESGPGLVAPSQSLSVTCTVSG |
| | | | VSLPDYGVSWIRQPPRKGLEWLGVIWGSETTYYNSALKSRLTIIKDNSKS |
| | | | QVFLKMNSLQTDDTAIYYCAKHYYYGGSYAMDYWGQGTSVTVSSESKY |
| | | | GPPCPPCPGQPREPQVYTLPPSRDELTKNQVSLTCLVKGFYPSDIAVE |
| | | | WESNGQPENNYKTTPPVLDSDGSFFLYSKLTVDKSRWQQGNVFSCSV |
| | | | MHEALHNAYTQKSLSLSPGKKDPKFWVLVVVGGVLACYSLLVTVAFIIF |
| | | | WVRSKRSRLLHSDYMNMTPRRPGPTRKHYQPYAPPRDFAAYRSRVKF |
| | | | SRSADAPAYQQGQNQLYNELNLGRREEYDVLDKRRGRDPEMGGKPR |
| | | | RKNPQEGLYNELQKDKMAEAYSEIGMKGERRRGKGHDGLYQGLSTAT |
| | | | KDTYDALHMQALPPR* |
| 21 | CD3-zeta | | RVKFSRSADAPAYQQGQNQLYNELNLGRREEYDVLDKRRGRDPEMGG |
| | Intracellular | | KPRRKNPQEGLYNELQKDKMAEAYSEIGMKGERRRGKGHDGLYQGLS |
| | Domain + STOP | | TATKDTYDALHMQALPPR* |
| 22 | eGFP | | MVSKGEELFTGVVPILVELDGDVNGHKFSVSGEGEGDATYGKLTLKFIC |
| | | | TTGKLPVPWPTLVTTLTYGVQCFSRYPDHMKQHDFFKSAMPEGYVQER |
| | | | TIFFKDDGNYKTRAEVKFEGDTLVNRIELKGIDFKEDGNILGHKLEYNYN |
| | | | SHNVYIMADKQKNGIKVNFKIRHNIEDGSVQLADHYQQNTPIGDGPVLLP |
| | | | DNHYLSTQSALSKDPNEKRDHMVLLEFVTAAGITLGMDELYK* |
| 23 | TRAC-Charité | Left homology | iqnpdpavyqlrdskssdksvclft |
| | | arm | |

Preferred Nucleic Acid Sequences of the Invention:

| | Homology- | Info | |
|---|---|---|---|
| SEQ | Directed DNA | HDR- | |
| ID | Repair (HDR) | Template | |
| NO | Template | Part | Nucleic acid sequence 5′-3′ |
| 24 | CD247- | Full Sequence | GGCAGGAGCTCTGTGCACAGAGAACAGCCTCATCTGCTCGCCTTGTTTC |
| | exon2_CD19CAR | | CACCTCCCCTCCCATTGCCCCAGGTTCTTTGGCCCCACAGCGGCCACAT |
| | (truncated) | | CTGCCGTTGGTGCCAATAGGTTTTCCAGGAGCTGGTTGAGGTGGGAGG |
| | | | GAGGGAGAGGGTTGTGATCAGGCTGAGGCATGGGGATTGGATATAGTC |
| | | | TCCGTGTCATGATTTATTTGGTCAGTCAGTCCTAGTGCCACCCTGGGGTA |
| | | | ATGGGGATGTGTTCTCGTCACCTTGGGCCTGGCTGACCAGCTTTATCTC |
| | | | TTGGCACAGAGGCACAGAGCTTTGGCCTGCTGGATCCCAAACTCTGCTA |
| | | | CCTGCTGGATGGAATCCTCTTCATCTATCGTGTCATTCTCACTGCCTTGT |
| | | | TCCTGAGAGCCACCAACTTCTCTCTTTTGAAGCAGGCCGGAGATGTGGA |
| | | | AGAAAATCCTGGGCCCATGGCTCTTCCTGTGACTGCCCTTCTGCTGCCC |
| | | | CTGGCTCTCCTGCTCCATGCTGCCCGGCCAGACATCCAGATGACACAGA |
| | | | CAACCAGCAGCCTCAGCGCCAGCCTGGGGGACAGAGTGACCATTAGCT |
| | | | GCCGGGCCTCTCAGGACATCAGCAAATACCTGAACTGGTACCAGCAGAA |
| | | | ACCAGATGGCACTGTCAAGCTGCTGATTTACCACACCTCCAGGCTCCAC |
| | | | AGCGGCGTGCCCAGTCGCTTCAGCGGCAGTGGGAGCGGGACAGATTAT |
| | | | TCCCTCACAATCTCCAACCTGGAGCAGGAAGATATTGCCACATACTTCTG |
| | | | CCAGCAAGGCAACACCCTGCCATACACATTTGGAGGCGGCACCAAATTG |
| | | | GAGATCACCGGCGGTGGTGGATCTGGAGGAGGGGGCAGCGGAGGTGG |
| | | | CGGCTCTGAGGTGAAACTGCAGGAGAGTGGCCCTGGCCTGGTGGCTCC |
| | | | CAGCCAGAGCCTTTCTGTCACCTGCACCGTGTCTGGGGTGTCCCTGCCT |
| | | | GACTATGGAGTCTCCTGGATCCGGCAGCCTCCAAGAAAAGGACTGGAAT |
| | | | GGCTGGGCGTCATCTGGGGAAGTGAGACCACCTACTATAATTCAGCCCT |
| | | | CAAGTCCCGGCTCACCATCATTAAGGACAACTCCAAATCCCAGGTGTTC |
| | | | CTGAAGATGAATTCTCTCCAGACTGATGACACAGCCATCTACTACTGTGC |
| | | | CAAGCATTATTATTACGGGGGGTCCTATGCCATGGACTACTGGGGCCAG |
| | | | GGGACCAGTGTCACTGTTTCTTCTGAAAGCAAGTATGGCCCCCCATGCC |
| | | | CTCCCTGCCCCGGACAGCCGCGGGAGCCCCAGGTCTACACCCTCCCAC |
| | | | CCTCCAGAGATGAGCTGACCAAGAACCAGGTTTCACTGACATGCCTGGT |
| | | | GAAGGGCTTCTACCCCTCTGACATTGCTGTGGAGTGGGAGAGCAATGG |
| | | | GCAGCCAGAGAACAACTACAAGACCACACCTCCTGTCCTGGACAGCGAC |
| | | | GGCTCCTTCTTCCTCTACAGTAAGCTCACTGTGGACAAGAGCCGCTGGC |
| | | | AGCAGGGCAATGTCTTCTCCTGCAGCGTGATGCACGAGGCCCTGCACA |
| | | | ATGCCTATACCCAGAAGTCACTCTCTCTGAGCCCTGGAAAGAAGGATCC |
| | | | CAAGTTTTGGGTCCTCGTGGTGGTCGGGGGTGTGCTGGCCTGCTACAG |
| | | | CTTGCTGGTCACAGTGGCCTTCATCATCTTCTGGGTGCGCTCCAAGAGG |
| | | | AGCCGGCTGCTTCACAGTGATTACATGAACATGACCCCCAGGAGGCCAG |
| | | | GACCCACCAGGAAGCACTACCAGCCCTACGCTCCCCCGCGGGACTTTG |
| | | | CTGCTTACCGCAGCAGAGTGAAGGTGGGTACCACTGGGCTTTGGGAGG |
| | | | AGGGCACGGGGTCCCCCACTTGATGGATGTTCAGAGGGGCCTTGGTCT |
| | | | TGGAAGGTCTCAAGCTCGGGTGGTGCCTGGGGCTTGGTATCCAGGAGC |
| | | | AAAGCAAGGACCAGCCAAGTGTGTGCCTTGAGTGGGCTGAGGAGGAGG |
| | | | TGGCAGTGTCTGGCTGAGATGGACAGGGTAGGAGGGAGAGCCTGGTGC |
| | | | TAGGCACCTCCATGACAAGCCGTACAAATGTGTGCACATCAGAGTGTCC |
| | | | CAGGGAAGGCGATGCTACTGGTGACAAAGGGGCTTACACTCAGGCAGA |
| | | | GGTCCTTCTTTCCAAGTGTGAATGAAGGCCATGTTAGCCTTTCTCTTGAA |
| | | | AAGGCCCTTTCCTCATCTGTAACTGGGGA |
| 25 | | Left Homology | GGCAGGAGCTCTGTGCACAGAGAACAGCCTCATCTGCTCGCCTTGTTTC |
| | | Arm | CACCTCCCCTCCCATTGCCCCAGGTTCTTTGGCCCCACAGCGGCCACAT |
| | | | CTGCCGTTGGTGCCAATAGGTTTTCCAGGAGCTGGTTGAGGTGGGAGG |
| | | | GAGGGAGAGGGTTGTGATCAGGCTGAGGCATGGGGATTGGATATAGTC |
| | | | TCCGTGTCATGATTTATTTGGTCAGTCAGTCCTAGTGCCACCCTGGGGTA |
| | | | ATGGGGATGTGTTCTCGTCACCTTGGGCCTGGCTGACCAGCTTTATCTC |
| | | | TTGGCACAGAGGCACAGAGCTTTGGCCTGCTGGATCCCAAACTCTGCTA |
| | | | CCTGCTGGATGGAATCCTCTTCATCTATCGTGTCATTCTCACTGCCTTGT |
| | | | TCCTGAGA |
| 26 | | P2A | GCCACCAACTTCTCTCTTTTGAAGCAGGCCGGAGATGTGGAAGAAAATC |
| | | | CTGGGCCC |
| 27 | | Membrane | ATGGCTCTTCCTGTGACTGCCCTTCTGCTGCCCCTGGCTCTCCTGCTCC |
| | | Export Signal | ATGCTGCCCGGCCA |
| | | (Leader) | |
| 28 | | FMC63VL | GACATCCAGATGACACAGACAACCAGCAGCCTCAGCGCCAGCCTGGGG |
| | | (Light Chain, | GACAGAGTGACCATTAGCTGCCGGGCCTCTCAGGACATCAGCAAATACC |
| | | Variable Part) | TGAACTGGTACCAGCAGAAACCAGATGGCACTGTCAAGCTGCTGATTTA |
| | | | CCACACCTCCAGGCTCCACAGCGGCGTGCCCAGTCGCTTCAGCGGCAG |
| | | | TGGGAGCGGGACAGATTATTCCCTCACAATCTCCAACCTGGAGCAGGAA |
| | | | GATATTGCCACATACTTCTGCCAGCAAGGCAACACCCTGCCATACACATT |
| | | | TGGAGGCGGCACCAAATTGGAGATCACC |
| 29 | | (G4S)3 Linker | GGCGGTGGTGGATCTGGAGGAGGGGGCAGCGGAGGTGGCGGCTCT |
| 30 | | FMC63VH | GAGGTGAAACTGCAGGAGAGTGGCCCTGGCCTGGTGGCTCCCAGCCAG |
| | | (Heavy Chain, | AGCCTTTCTGTCACCTGCACCGTGTCTGGGGTGTCCCTGCCTGACTATG |
| | | Variable Part) | GAGTCTCCTGGATCCGGCAGCCTCCAAGAAAAGGACTGGAATGGCTGG |
| | | | GCGTCATCTGGGGAAGTGAGACCACCTACTATAATTCAGCCCTCAAGTC |
| | | | CCGGCTCACCATCATTAAGGACAACTCCAAATCCCAGGTGTTCCTGAAG |
| | | | ATGAATTCTCTCCAGACTGATGACACAGCCATCTACTACTGTGCCAAGCA |
| | | | TTATTATTACGGCGGGTCCTATGCCATGGACTACTGGGGCCAGGGGACC |
| | | | AGTGTCACTGTTTCTTCT |
| 31 | | IgG4 Hinge | GAAAGCAAGTATGGCCCCCCATGCCCTCCCTGCCCC |
| 32 | | IgG1 CH3 | GGACAGCCGCGGGAGCCCCAGGTCTACACCCTCCCACCCTCCAGAGAT |
| | | Domain | GAGCTGACCAAGAACCAGGTTTCACTGACATGCCTGGTGAAGGGCTTCT |
| | | (Hinge) | ACCCCTCTGACATTGCTGTGGAGTGGGAGAGCAATGGGCAGCCAGAGA |
| | | | ACAACTACAAGACCACACCTCCTGTCCTGGACAGCGACGGCTCCTTCTT |
| | | | CCTCTACAGTAAGCTCACTGTGGACAAGAGCCGCTGGCAGCAGGGCAA |
| | | | TGTCTTCTCCTGCAGCGTGATGCACGAGGCCCTGCACAATGCCTATACC |
| | | | CAGAAGTCACTCTCTCTGAGCCCTGGAAAG |
| 33 | | Linker | AAGGATCCCAAG |
| 34 | | CD28 | TTTTGGGTCCTCGTGGTGGTCGGGGGTGTGCTGGCCTGCTACAGCTTG |
| | | Transmembrane | CTGGTCACAGTGGCCTTCATCATCTTCTGGGTG |
| | | Domain | |
| 35 | | CD28 | CGCTCCAAGAGGAGCCGGCTGCTTCACAGTGATTACATGAACATGACCC |
| | | Intracellular | CCAGGAGGCCAGGACCCACCAGGAAGCACTACCAGCCCTACGCTCCCC |
| | | Domain | CGCGGGACTTTGCTGCTTACCGCAGC |
| 36 | | Right | AGAGTGAAGGTGGGTACCACTGGGCTTTGGGAGGAGGGCACGGGGTC |
| | | Homology | CCCCACTTGATGGATGTTCAGAGGGGCCTTGGTCTTGGAAGGTCTCAAG |
| | | Arm | CTCGGGTGGTGCCTGGGGCTTGGTATCCAGGAGCAAAGCAAGGACCAG |
| | | | CCAAGTGTGTGCCTTGAGTGGGCTGAGGAGGAGGTGGCAGTGTCTGGC |
| | | | TGAGATGGACAGGGTAGGAGGGAGAGCCTGGTGCTAGGCACCTCCATG |
| | | | ACAAGCCGTACAAATGTGTGCACATCAGAGTGTCCCAGGGAAGGCGATG |
| | | | CTACTGGTGACAAAGGGGCTTACACTCAGGCAGAGGTCCTTCTTTCCAA |
| | | | GTGTGAATGAAGGCCATGTTAGCCTTTCTCTTGAAAAGGCCCTTTCCTCA |
| | | | TCTGTAACTGGGGA |
| 37 | CD247- | Full Sequence | GGCAGGAGCTCTGTGCACAGAGAACAGCCTCATCTGCTCGCCTTGTTTC |
| | exon2_ | | CACCTCCCCTCCCATTGCCCCAGGTTCTTTGGCCCCACAGCGGCCACAT |
| | CD19CAR | | CTGCCGTTGGTGCCAATAGGTTTTCCAGGAGCTGGTTGAGGTGGGAGG |
| | (full) | | GAGGGAGAGGGTTGTGATCAGGCTGAGGCATGGGGATTGGATATAGTC |
| | | | TCCGTGTCATGATTTATTTGGTCAGTCAGTCCTAGTGCCACCCTGGGGTA |
| | | | ATGGGGATGTGTTCTCGTCACCTTGGGCCTGGCTGACCAGCTTTATCTC |
| | | | TTGGCACAGAGGCACAGAGCTTTGGCCTGCTGGATCCCAAACTCTGCTA |
| | | | CCTGCTGGATGGAATCCTCTTCATCTATCGTGTCATTCTCACTGCCTTGT |
| | | | TCCTGAGAGCCACCAACTTCTCTCTTTTGAAGCAGGCCGGAGATGTGGA |
| | | | AGAAAATCCTGGGCCCATGGCTCTTCCTGTGACTGCCCTTCTGCTGCCC |
| | | | CTGGCTCTCCTGCTCCATGCTGCCCGGCCAGACATCCAGATGACACAGA |
| | | | CAACCAGCAGCCTCAGCGCCAGCCTGGGGGACAGAGTGACCATTAGCT |
| | | | GCCGGGCCTCTCAGGACATCAGCAAATACCTGAACTGGTACCAGCAGAA |
| | | | ACCAGATGGCACTGTCAAGCTGCTGATTTACCACACCTCCAGGCTCCAC |
| | | | AGCGGCGTGCCCAGTCGCTTCAGCGGCAGTGGGAGCGGGACAGATTAT |
| | | | TCCCTCACAATCTCCAACCTGGAGCAGGAAGATATTGCCACATACTTCTG |
| | | | CCAGCAAGGCAACACCCTGCCATACACATTTGGAGGCGGCACCAAATTG |
| | | | GAGATCACCGGCGGTGGTGGATCTGGAGGAGGGGGCAGCGGAGGTGG |
| | | | CGGCTCTGAGGTGAAACTGCAGGAGAGTGGCCCTGGCCTGGTGGCTCC |
| | | | CAGCCAGAGCCTTTCTGTCACCTGCACCGTGTCTGGGGTGTCCCTGCCT |
| | | | GACTATGGAGTCTCCTGGATCCGGCAGCCTCCAAGAAAAGGACTGGAAT |
| | | | GGCTGGGCGTCATCTGGGGAAGTGAGACCACCTACTATAATTCAGCCCT |
| | | | CAAGTCCCGGCTCACCATCATTAAGGACAACTCCAAATCCCAGGTGTTC |
| | | | CTGAAGATGAATTCTCTCCAGACTGATGACACAGCCATCTACTACTGTGC |
| | | | CAAGCATTATTATTACGGGGGGTCCTATGCCATGGACTACTGGGGCCAG |
| | | | GGGACCAGTGTCACTGTTTCTTCTGAAAGCAAGTATGGCCCCCCATGCC |
| | | | CTCCCTGCCCCGGACAGCCGCGGGAGCCCCAGGTCTACACCCTCCCAC |
| | | | CCTCCAGAGATGAGCTGACCAAGAACCAGGTTTCACTGACATGCCTGGT |
| | | | GAAGGGCTTCTACCCCTCTGACATTGCTGTGGAGTGGGAGAGCAATGG |
| | | | GCAGCCAGAGAACAACTACAAGACCACACCTCCTGTCCTGGACAGCGAC |
| | | | GGCTCCTTCTTCCTCTACAGTAAGCTCACTGTGGACAAGAGCCGCTGGC |
| | | | AGCAGGGCAATGTCTTCTCCTGCAGCGTGATGCACGAGGCCCTGCACA |
| | | | ATGCCTATACCCAGAAGTCACTCTCTCTGAGCCCTGGAAAGAAGGATCC |
| | | | CAAGTTTTGGGTCCTCGTGGTGGTCGGGGGTGTGCTGGCCTGCTACAG |
| | | | CTTGCTGGTCACAGTGGCCTTCATCATCTTCTGGGTGCGCTCCAAGAGG |
| | | | AGCCGGCTGCTTCACAGTGATTACATGAACATGACCCCCAGGAGGCCAG |
| | | | GACCCACCAGGAAGCACTACCAGCCCTACGCTCCCCCGCGGGACTTTG |
| | | | CTGCTTACCGCAGCAGGGTCAAATTTTCTAGATCTGCAGATGCGCCGGC |
| | | | CTATCAACAAGGCCAGAACCAGCTCTATAACGAGCTCAATCTAGGACGA |
| | | | AGAGAGGAGTACGATGTTTTGGACAAGAGACGTGGCCGGGACCCTGAG |
| | | | ATGGGGGGAAAGCCGAGAAGGAAGAACCCTCAGGAAGGCCTGTACAAT |
| | | | GAACTGCAGAAAGATAAGATGGCGGAGGCCTACAGTGAGATTGGGATG |
| | | | AAAGGCGAGCGCCGGAGGGGCAAGGGGCACGATGGCCTTTACCAGGG |
| | | | TCTCAGTACAGCCACCAAGGACACCTACGACGCCCTTCACATGCAGGCC |
| | | | CTGCCCCCTCGCTAATAAGAATTCTAACTAGAGCTCGCTGATCAGCCTC |
| | | | GACTGTGCCTTCTAGTTGCCAGCCATCTGTTGTTTGCCCCTCCCCCGTG |
| | | | CCTTCCTTGACCCTGGAAGGTGCCACTCCCACTGTCCTTTCCTAATAAAA |
| | | | TGAGGAAATTGCATCGCATTGTCTGAGTAGGTGTCATTCTATTCTGGGG |
| | | | GGTGGGGTGGGGCAGGACAGCAAGGGGGAGGATTGGGAAGAGAATAG |
| | | | CAGGCATGCTGGGGAGCGGCCGCAGAGTGAAGGTGGGTACCACTGGG |
| | | | CTTTGGGAGGAGGGCACGGGGTCCCCCACTTGATGGATGTTCAGAGGG |
| | | | GCCTTGGTCTTGGAAGGTCTCAAGCTCGGGTGGTGCCTGGGGCTTGGT |
| | | | ATCCAGGAGCAAAGCAAGGACCAGCCAAGTGTGTGCCTTGAGTGGGCT |
| | | | GAGGAGGAGGTGGCAGTGTCTGGCTGAGATGGACAGGGTAGGAGGGA |
| | | | GAGCCTGGTGCTAGGCACCTCCATGACAAGCCGTACAAATGTGTGCACA |
| | | | TCAGAGTGTCCCAGGGAAGGCGATGCTACTGGTGACAAAGGGGCTTAC |
| | | | ACTCAGGCAGAGGTCCTTCTTTCCAAGTGTGAATGAAGGCCATGTTAGC |
| | | | CTTTCTCTTGAAAAGGCCCTTTCCTCATCTGTAACTGGGGA |
| 38 | CD247- | Full Sequence | ATTGTCCTCAGAGTCCCCGGAGGAAGGGATCCTAGAGAATACTCATGTC |
| | exon3_ | | CCCGGGGGCGCAGACCAAGAACCTTATACCTCTGCGCACGCGCGAGGG |
| | CD19CAR | | CGCTAGCCCGGGAAGAATAAACTCCAGCGGGTCCTGGGCCAGCGTGAT |
| | (truncated) | | GAATGCACGTGTCACAGGGGGGAGGAAAAAGGACAACTGGGCTCAGAA |
| | | | AAAGGGGCTGCCACGCCCCGGCCCCTGCAAGGGCTCAGCCCCAGGAG |
| | | | GCAGGGCAGCATTGTTAGTTGCCAAGGAGCGGAGTAGGGCTGTCCCTC |
| | | | GGGTCGCCGCGCGCCCTGGCACCCCGGCTCCCCTGCGCGCCCTCGCG |
| | | | GAGCAGTAACGCGCTTTGCTTTCTGTGTTGCAGTTCAGCAGGAGCGCAG |
| | | | ACGCCCCCGCGTACGCCACCAACTTCTCTCTTTTGAAGCAGGCCGGAGA |
| | | | TGTGGAAGAAAATCCTGGGCCCATGGCTCTTCCTGTGACTGCCCTTCTG |
| | | | CTGCCCCTGGCTCTCCTGCTCCATGCTGCCCGGCCAGACATCCAGATGA |
| | | | CACAGACAACCAGCAGCCTCAGCGCCAGCCTGGGGGACAGAGTGACCA |
| | | | TTAGCTGCCGGGCCTCTCAGGACATCAGCAAATACCTGAACTGGTACCA |
| | | | GCAGAAACCAGATGGCACTGTCAAGCTGCTGATTTACCACACCTCCAGG |
| | | | CTCCACAGCGGCGTGCCCAGTCGCTTCAGCGGCAGTGGGAGCGGGAC |
| | | | AGATTATTCCCTCACAATCTCCAACCTGGAGCAGGAAGATATTGCCACAT |
| | | | ACTTCTGCCAGCAAGGCAACACCCTGCCATACACATTTGGAGGCGGCAC |
| | | | CAAATTGGAGATCACCGGCGGTGGTGGATCTGGAGGAGGGGGCAGCG |
| | | | GAGGTGGCGGCTCTGAGGTGAAACTGCAGGAGAGTGGCCCTGGCCTG |
| | | | GTGGCTCCCAGCCAGAGCCTTTCTGTCACCTGCACCGTGTCTGGGGTGT |
| | | | CCCTGCCTGACTATGGAGTCTCCTGGATCCGGCAGCCTCCAAGAAAAGG |
| | | | ACTGGAATGGCTGGGCGTCATCTGGGGAAGTGAGACCACCTACTATAAT |
| | | | TCAGCCCTCAAGTCCCGGCTCACCATCATTAAGGACAACTCCAAATCCC |
| | | | AGGTGTTCCTGAAGATGAATTCTCTCCAGACTGATGACACAGCCATCTAC |
| | | | TACTGTGCCAAGCATTATTATTACGGGGGGTCCTATGCCATGGACTACTG |
| | | | GGGCCAGGGGACCAGTGTCACTGTTTCTTCTGAAAGCAAGTATGGCCCC |
| | | | CCATGCCCTCCCTGCCCCGGACAGCCGCGGGAGCCCCAGGTCTACACC |
| | | | CTCCCACCCTCCAGAGATGAGCTGACCAAGAACCAGGTTTCACTGACAT |
| | | | GCCTGGTGAAGGGCTTCTACCCCTCTGACATTGCTGTGGAGTGGGAGA |
| | | | GCAATGGGCAGCCAGAGAACAACTACAAGACCACACCTCCTGTCCTGGA |
| | | | CAGCGACGGCTCCTTCTTCCTCTACAGTAAGCTCACTGTGGACAAGAGC |
| | | | CGCTGGCAGCAGGGCAATGTCTTCTCCTGCAGCGTGATGCACGAGGCC |
| | | | CTGCACAATGCCTATACCCAGAAGTCACTCTCTCTGAGCCCTGGAAAGA |
| | | | AGGATCCCAAGTTTTGGGTCCTCGTGGTGGTCGGGGGTGTGCTGGCCT |
| | | | GCTACAGCTTGCTGGTCACAGTGGCCTTCATCATCTTCTGGGTGCGCTC |
| | | | CAAGAGGAGCCGGCTGCTTCACAGTGATTACATGAACATGACCCCCAGG |
| | | | AGGCCAGGACCCACCAGGAAGCACTACCAGCCCTACGCTCCCCCGCGG |
| | | | GACTTTGCTGCTTACCGCAGCAGGGTCAAATTTTCTAGATCTGCAGATGC |
| | | | GCCGGCCTATCAACAAGGCCAGAACCAGCTCTATAACGTAAGTCAGCCT |
| | | | CGCCGTAGACCCTCCGGAAAGGAAGGGCAGCCTCCTCCTCGGACCTAG |
| | | | GGAACGCGCCCGCCAGAGTCTTGGGTTTAGGTTTGGTCCCTAGCTATCA |
| | | | CCTCTGCGACCCGGCTGCAACTTGTTTAACCTCAGTTTTCTAACCCACAA |
| | | | AACAGGTGTAGTAATTGTACCTGTCTCAAAGGGCGCCATAAAGATTACAT |
| | | | GCTTTAATGTCTTGATGCATGGAACGAGCTTAGTCTAGTGCCTGCCACTA |
| | | | GTGAGCCCCCAAGCCATCAGCAATTTGCAAAGGCTAGAGGGGGGTGGA |
| | | | GAAGGAATGTATTAGAATCTTCCCAGAGGAGGGGAAAGCGATTTCACAC |
| | | | TCCCTGTGCAATGGGACTG |
| 39 | | Left Homology | ATTGTCCTCAGAGTCCCCGGAGGAAGGGATCCTAGAGAATACTCATGTC |
| | | Arm | CCCGGGGGCGCAGACCAAGAACCTTATACCTCTGCGCACGCGCGAGGG |
| | | | CGCTAGCCCGGGAAGAATAAACTCCAGCGGGTCCTGGGCCAGCGTGAT |
| | | | GAATGCACGTGTCACAGGCGGGAGGAAAAAGGACAACTGGGCTCAGAA |
| | | | AAAGGGGCTGCCACGCCCCGGCCCCTGCAAGGGCTCAGCCCCAGGAG |
| | | | GCAGGGCAGCATTGTTAGTTGCCAAGGAGCGGAGTAGGGCTGTCCCTC |
| | | | GGGTCGCCGCGCGCCCTGGCACCCCGGCTCCCCTGCGCGCCCTCGCG |
| | | | GAGCAGTAACGCGCTTTGCTTTCTGTGTTGCAGTTCAGCAGGAGCGCAG |
| | | | ACGCCCCCGCGTAC |
| 40 | | CAR body | SEQ ID NO 26-35 |
| 41 | | truncated | AGGGTCAAATTTTCTAGATCTGCA |
| | | CD3-zeta | |
| 42 | | Right | GATGCGCCGGCCTATCAACAAGGCCAGAACCAGCTCTATAACGTAAGTC |
| | | Homology | AGCCTCGCCGTAGACCCTCCGGAAAGGAAGGGCAGCCTCCTCCTCGGA |
| | | Arm with | CCTAGGGAACGCGCCCGCCAGAGTCTTGGGTTTAGGTTTGGTCCCTAG |
| | | Silent | CTATCACCTCTGCGACCCGGCTGCAACTTGTTTAACCTCAGTTTTCTAAC |
| | | Mutations to | CCACAAAACAGGTGTAGTAATTGTACCTGTCTCAAAGGGCGCCATAAAG |
| | | Prevent Cas9 | ATTACATGCTTTAATGTCTTGATGCATGGAACGAGCTTAGTCTAGTGCCT |
| | | Re-Cutting | GCCACTAGTGAGCCCCCAAGCCATCAGCAATTTGCAAAGGCTAGAGGG |
| | | | GGGTGGAGAAGGAATGTATTAGAATCTTCCCAGAGGAGGGGAAAGCGA |
| | | | TTTCACACTCCCTGTGCAATGGGACTG |
| 43 | CD247-exon3_ | Right | CAAGGCCAGAACCAGCTCTATAACGTAAGTCAGCCTCGCCGTAGACCCT |
| | | Homology | CCGGAAAGGAAGGGCAGCCTCCTCCTCGGACCTAGGGAACGCGCCCG |
| | | Arm | CCAGAGTCTTGGGTTTAGGTTTGGTCCCTAGCTATCACCTCTGCGACCC |
| | | | GGCTGCAACTTGTTTAACCTCAGTTTTCTAACCCACAAAACAGGTGTAGT |
| | | | AATTGTACCTGTCTCAAAGGGCGCCATAAAGATTACATGCTTTAATGTCT |
| | | | TGATGCATGGAACGAGCTTAGTCTAGTGCCTGCCACTAGTGAGCCCCCA |
| | | | AGCCATCAGCAATTTGCAAAGGCTAGAGGGGGGTGGAGAAGGAATGTAT |
| | | | TAGAATCTTCCCAGAGGAGGGGAAAGCGATTTCACACTCCCTGTGCAAT |
| | | | GGGACTG |
| 44 | CD3-zeta | | AGGGTCAAATTTTCTAGATCTGCAGATGCGCCGGCCTATCAACAAGGCC |
| | Intracellular | | AGAACCAGCTCTATAACGAGCTCAATCTAGGACGAAGAGAGGAGTACGA |
| | Domain + | | TGTTTTGGACAAGAGACGTGGCCGGGACCCTGAGATGGGGGGAAAGCC |
| | STOP | | GAGAAGGAAGAACCCTCAGGAAGGCCTGTACAATGAACTGCAGAAAGAT |
| | | | AAGATGGCGGAGGCCTACAGTGAGATTGGGATGAAAGGCGAGCGCCGG |
| | | | AGGGGCAAGGGGCACGATGGCCTTTACCAGGGTCTCAGTACAGCCACC |
| | | | AAGGACACCTACGACGCCCTTCACATGCAGGCCCTGCCCCCTCGCTAA |
| 45 | bGH Poly-A | | TAAGAATTCTAACTAGAGCTCGCTGATCAGCCTCGACTGTGCCTTCTAGT |
| | Signal | | TGCCAGCCATCTGTTGTTTGCCCCTCCCCCGTGCCTTCCTTGACCCTGG |
| | (Extended) | | AAGGTGCCACTCCCACTGTCCTTTCCTAATAAAATGAGGAAATTGCATCG |
| | | | CATTGTCTGAGTAGGTGTCATTCTATTCTGGGGGGTGGGGTGGGGCAG |
| | | | GACAGCAAGGGGGAGGATTGGGAAGAGAATAGCAGGCATGCTGGGGA |
| | | | GCGGCCGC |
| 46 | eGFP | | ATGGTATCCAAGGGTGAAGAATTGTTCACTGGCGTAGTTCCTATCTTGGT |
| | | | GGAGCTCGACGGAGATGTCAATGGACACAAGTTTTCAGTCAGTGGCGAA |
| | | | GGTGAGGGTGATGCAACTTACGGGAAGCTTACACTGAAATTCATTTGTA |
| | | | CTACTGGCAAGTTGCCGGTGCCATGGCCCACGCTGGTAACGACCCTTAC |
| | | | CTACGGTGTCCAATGTTTTTCTCGATACCCCGATCATATGAAACAGCATG |
| | | | ACTTTTTTAAATCTGCTATGCCCGAGGGATACGTACAGGAAAGGACTATA |
| | | | TTTTTTAAGGACGATGGTAACTATAAGACCCGCGCCGAAGTCAAGTTTGA |
| | | | GGGAGATACTCTTGTTAATCGAATCGAGCTCAAGGGGATAGATTTTAAG |
| | | | GAGGATGGCAACATTCTCGGCCACAAGCTGGAGTACAATTACAATAGTC |
| | | | ACAATGTTTACATAATGGCTGATAAGCAAAAAAACGGCATCAAAGTTAAT |
| | | | TTTAAAATTAGGCATAATATAGAAGACGGTTCAGTTCAACTCGCCGACCA |
| | | | CTACCAGCAGAATACCCCTATTGGCGATGGGCCGGTTCTCCTGCCTGAC |
| | | | AACCATTACCTCTCAACACAGTCTGCTTTGAGTAAAGACCCTAACGAGAA |
| | | | GCGAGATCATATGGTCTTGCTCGAGTTCGTAACGGCCGCCGGTATAACC |
| | | | CTGGGCATGGATGAGCTCTACAAATAA |
| 47 | TRAC-Charité | Left Homology | CCTATTAAATAAAAGAATAAGCAGTATTATTAAGTAGCCCTGCATTTCAGG |
| | | Arm | TTTCCTTGAGTGGCAGGCCAGGCCTGGCCGTGAACGTTCACTGAAATCA |
| | | | TGGCCTCTTGGCCAAGATTGATAGCTTGTGCCTGTCCCTGAGTCCCAGT |
| | | | CCATCACGAGCAGCTGGTTTCTAAGATGCTATTTCCCGTATAAAGCATGA |
| | | | GACCGTGACTTGCCAGCCCCACAGAGCCCCGCCCTTGTCCATCACTGG |
| | | | CATCTGGACTCCAGCCTGGGTTGGGGCAAAGAGGGAAATGAGATCATGT |
| | | | CCTAACCCTGATCCTCTTGTCCCACAGATATCCAGAACCCTGACCCTGC |
| | | | CGTGTACCAGCTGAGAGACTCTAAATCCAGTGACAAGTCTGTCTGCCTA |
| | | | TTCACC |
| 48 | | Right | ATTTTGATTCTCAAACAAATGTGTCACAAAGTAAGGATTCTGATGTGTATA |
| | | Homology | TCACAGACAAAACTGTGCTAGACATGAGGTCTATGGACTTCAAGAGCAA |
| | | Arm | CAGTGCTGTGGCCTGGAGCAACAAATCTGACTTTGCATGTGCAAACGCC |
| | | | TTCAACAACAGCATTATTCCAGAAGACACCTTCTTCCCCAGCCCAGGTAA |
| | | | GGGCAGCTTTGGTGCCTTCGCAGGCTGTTTCCTTGCTTCAGGAATGGCC |
| | | | AGGTTCTGCCCAGAGCTCTGGTCAATGATGTCTAAAACTCCTCTGATTGG |
| | | | TGGTCTCGGCCTTATCCATTGCCACCAAAACCCTCTTTTTACTAAGAAAC |
| | | | AGTGAGCCTTGTTCTGGCAGTCCAGAGAATGACACGGGAAAAAAGCAGA |
| | | | TG |
| 49 | AAVS1_EF1a- | Left Homology | ACCGTTTTTCTGGACAACCCCAAAGTACCCCGTCTCCCTGGCTTTAGCC |
| | LTR_CD19CAR | Arm | ACCTCTCCATCCTCTTGCTTTCTTTGCCTGGACACCCCGTTCTCCTGTGG |
| | | | ATTCGGGTCACCTCTCACTCCTTTCATTTGGGCAGCTCCCCTACCCCCCT |
| | | | TACCTCTCTAGTCTGTGCTAGCTCTTCCAGCCCCCTGTCATGGCATCTTC |
| | | | CAGGGGTCCGAGAGCTCAGCTAGTCTTCTTCCTCCAACCCGGGCCCCTA |
| | | | TGTCCACTTCAGGACAGCATGTTTGCTGCCTCCAGGGATCCTGTGTCCC |
| | | | CGAGCTGGGACCACCTTATATTCCCAGGGCCGGTTAATGTGGCTCTGGT |
| | | | TCTGGGTACTTTTATCTGTCCCCTCCACCCCACAGTGGGGCCACTAGGG |
| | | | ACAG |
| 50 | | EF1a-LTR | GTTTATTACAGGGACAGCAGAGATCCAGTTTGGGGATCAATTGCATGAA |
| | | Promotor | GAATCTGCTTAGGGTTAGGCGTTTTGCGCTGCTTCGCGAGGATCTGCGA |
| | | | TCGCTCCGGTGCCCGTCAGTGGGCAGAGCGCACATCGCCCACAGTCCC |
| | | | CGAGAAGTTGGGGGGAGGGGTCGGCAATTGAACCGGTGCCTAGAGAAG |
| | | | GTGGCGCGGGGTAAACTGGGAAAGTGATGTCGTGTACTGGCTCCGCCT |
| | | | TTTTCCCGAGGGTGGGGGAGAACCGTATATAAGTGCAGTAGTCGCCGTG |
| | | | AACGTTCTTTTTCGCAACGGGTTTGCCGCCAGAACACAGCTGAAGCTTC |
| | | | GAGGGGCTCGCATCTCTCCTTCACGCGCCCGCCGCCCTACCTGAGGCC |
| | | | GCCATCCACGCCGGTTGAGTCGCGTTCTGCCGCCTCCCGCCTGTGGTG |
| | | | CCTCCTGAACTGCGTCCGCCGTCTAGGTAAGTTTAAAGCTCAGGTCGAG |
| | | | ACCGGGCCTTTGTCCGGCGCTCCCTTGGAGCCTACCTAGACTCAGCCG |
| | | | GCTCTCCACGCTTTGCCTGACCCTGCTTGCTCAACTCTACGTCTTTGTTT |
| | | | CGTTTTCTGTTCTGCGCCGTTACAGATCCAAGCTGTGACCGGCGCCTAC |
| | | | GGCTAGCGCCGCCACC |
| 51 | | Right | GATTGGTGACAGAAAAGCCCCATCCTTAGGCCTCCTCCTTCCTAGTCTC |
| | | Homology | CTGATATTGGGTCTAACCCCCACCTCCTGTTAGGCAGATTCCTTATCTGG |
| | | Arm | TGACACACCCCCATTTCCTGGAGCCATCTCTCTCCTTGCCAGAACCTCTA |
| | | | AGGTTTGCTTACGATGGAGCCAGAGAGGATCCTGGGAGGGAGAGCTTG |
| | | | GCAGGGGGTGGGAGGGAAGGGGGGGATGCGTGACCTGCCCGGTTCTC |
| | | | AGTGGCCACCCTGCGCTACCCTCTCCCAGAACCTGAGCTGCTCTGACG |
| | | | CGGCCGTCTGGTGCGTTTCACTGATCCTGGTGCTGCAGCTTCCTTACAC |
| | | | TTCCCAAGAGGAGAAGCAGTTTGGAAAAACAAAATCAGAATAAGTTGGTC |
| | | | CTGAGTTCT |

Other sequences as used in specific embodiments and presented in the Examples share the nucleic acid and amino acid sequences as presented below. A full Sequence for a particular construct can be assembled according to the Sequence IDs given.

| | Preferred amino acid | Preferred nucleic acid |
|---|---|---|
| | sequences | sequences |
| CD247-exon2_CD19CAR(full) | SEQ ID NO 2-12, 21 | SEQ ID NO 25-35, 44, 45, 36 |
| CD247-exon2_CD19CAR(short/ | SEQ ID NO 1 (complete) | SEQ ID NO 24 (complete) |
| truncated) | or: SEQ ID NO 2-13 | or SEQ ID NO 25-36 |
| | (individual components) | (individual components) |
| CD247-exon2_eGFP | SEQ ID NO 2, 3, 22 | SEQ ID NO 25, 26, 46, 45, 36 |
| CD247-exon3_CD19CAR(full) | SEQ ID NO 16, 3-12, 21 | SEQ ID NO 39, 26-35, |
| | | 44, 45, 43 |
| CD247-exon3_CD19CAR(short/ | SEQ ID NO 15 (complete) | SEQ ID NO 38 (complete) or: |
| truncated) | or: SEQ ID NO 16, 3-12, | SEQ ID NO 39, 26-35, 41, 42 |
| | 18-19 (individual components) | (individual components) |
| CD247-exon3_eGFP | SEQ ID NO 16, 3, 22 | SEQ ID NO 39, 26, 46, 45, 43 |
| TRAC-Charité_CD19CAR(full) | SEQ ID NO 23, 3-12, 21 | SEQ ID NO 47, 26-35, |
| | | 44, 45, 48 |
| TRAC-Charité_eGFP | SEQ ID 23, 3, 22 | SEQ ID NO 47, 26, 46, 45, 48 |
| AAVS1_EF1a- | SEQ ID NO 4-12, 21 | SEQ ID NO 49, 50, 27-35, |
| LTR_CD19CAR(full) | | 44, 45, 51 |
| CD247-exon2_CD19CAR- | SEQ ID NO 59 | SEQ ID NO 60 |
| Y(truncated) (FMC63.28) | | |
| AAVS1_CMV_CD19CAR-Y | SEQ ID NO 61 | SEQ ID NO 62 |
| (FMC63.28.z) | | |
| AAVS1_CMV_CD19CAR-K | SEQ ID NO 63 | SEQ ID NO 64 |
| (FMC63.CD8a.BB.z) | | |

Full sequences of SEQ ID NO 59 to SEQ ID NO 64 are disclosed in the sequence listing accompanying the present application.

In specific embodiments, CAR nucleic acid sequence constructs of the prior art used in the Examples for comparison purposes, i.e. nucleic acid constructs encoding the TRAC-CD19CAR(full) or TRAC-eGFP polypeptide published by Roth et al. 2018 have been designed according to PMID: 29995861 and the respective SEQ IDs for the CD19CAR and eGFP listed above.

Preferred sgRNA Nucleic Acid Sequences of the Invention:

| Location & | | | | | | |
|---|---|---|---|---|---|---|
| corresponding | | | Ctrl: TRAC | Ctrl: TRAC | | B2M- |
| donor template | CD247 | CD247 | Exon 1- | Exon 1- | Ctrl: | specific |
| names: | Exon 2 | Exon 3 | Marson Lab | Charité | AAVS1 | RNP |
| SpCas9 sgRNAs | CD247 | CD247 | TRAC (Roth | TRAC sg 1 | AAVS1 (Mali | B2M KO sg: |
| | sg2.1: | sg1: | et al 2018): | GGGAAUC | et al 2013) | GGCCACG |
| | gaUggaaUc | gacgccccc | AGAGUCU | AAAAUCGG | GGGGCCA | GAGCGAG |
| | CUCUUcaUc | g | CUCAGCU | UGAAU | CUAGGGA | ACAUCU |
| | Ua | cgUaccagc | GGUACA | (SEQ ID NO | CAGGAU | (SEQ ID NO |
| | (SEQ ID NO | a (SEQ ID | (SEQ ID NO | 55) | (SEQ ID NO | 65) |
| | 52) | NO 53) | 54) | | 56) | |
| | CD247 | | | | | |
| | sg2.2: | | | | | |
| | UGGUACC | | | | | |
| | CACCUUCA | | | | | |
| | CUCUC | | | | | |
| | (SEQ ID NO | | | | | |
| | 57) | | | | | |
| | CD247 | | | | | |
| | sg2.4: | | | | | |
| | UgccUUgU | | | | | |
| | UccUgagag | | | | | |
| | Uga | | | | | |
| | (SEQ ID NO | | | | | |
| | 58) | | | | | |

sgRNA sequences and respective locations/donor templates. [sg = single guide RNA, AAVS1 = Adeno-associated virus integration site 1]]

### FIGURES

The invention is demonstrated by way of the example through the figures disclosed herein. The figures provided represent particular, non-limiting embodiments and are not intended to limit the scope of the invention.

### SHORT DESCRIPTION OF THE FIGURES

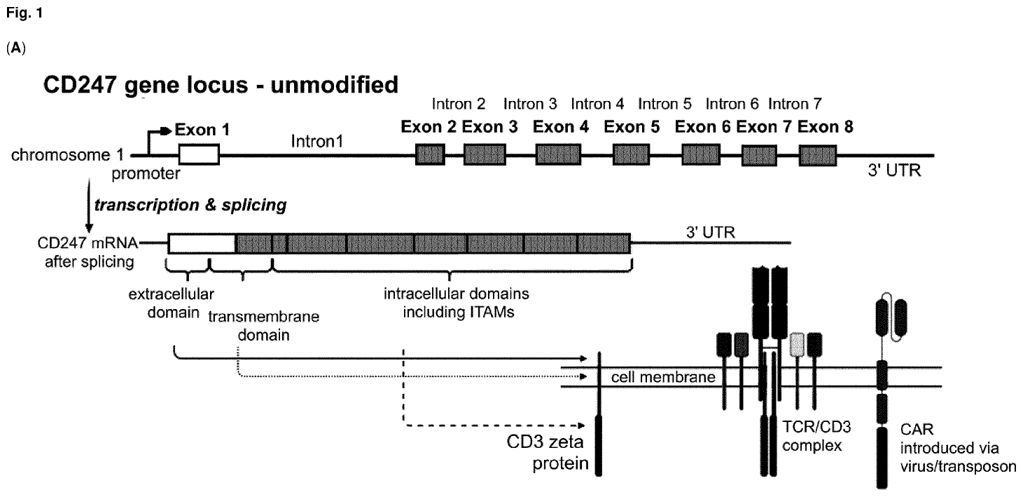

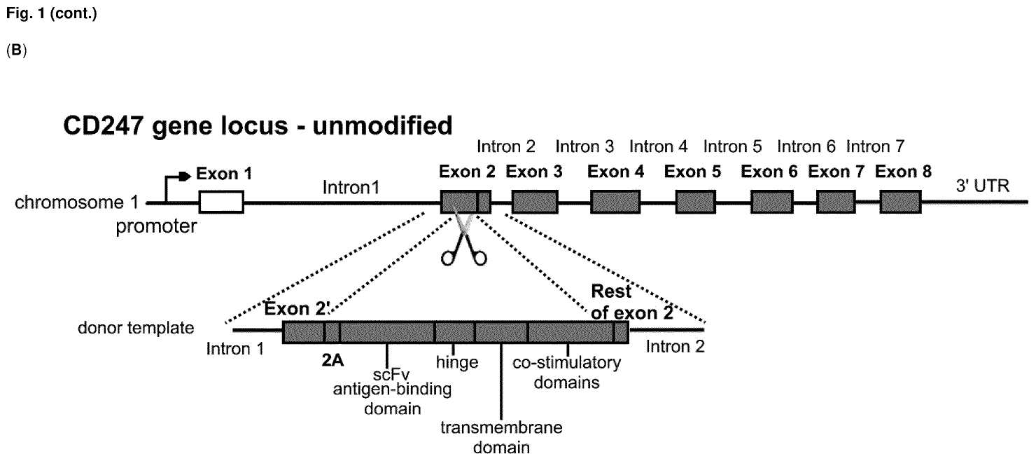

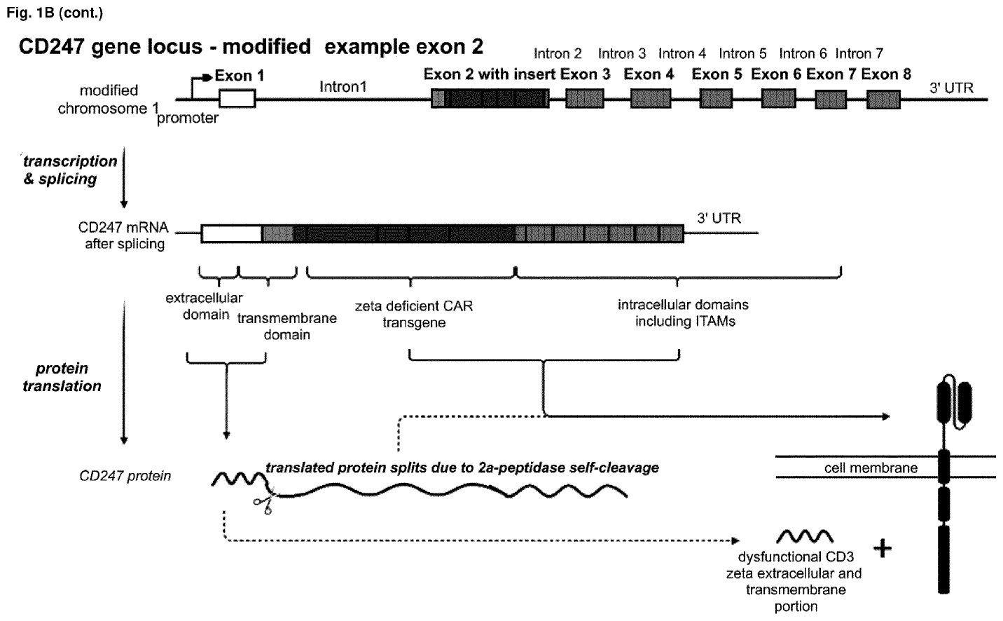

FIG. 1: New strategy fuses zeta-deficient CAR transgenes to endogenous CD3 zeta on genetic level

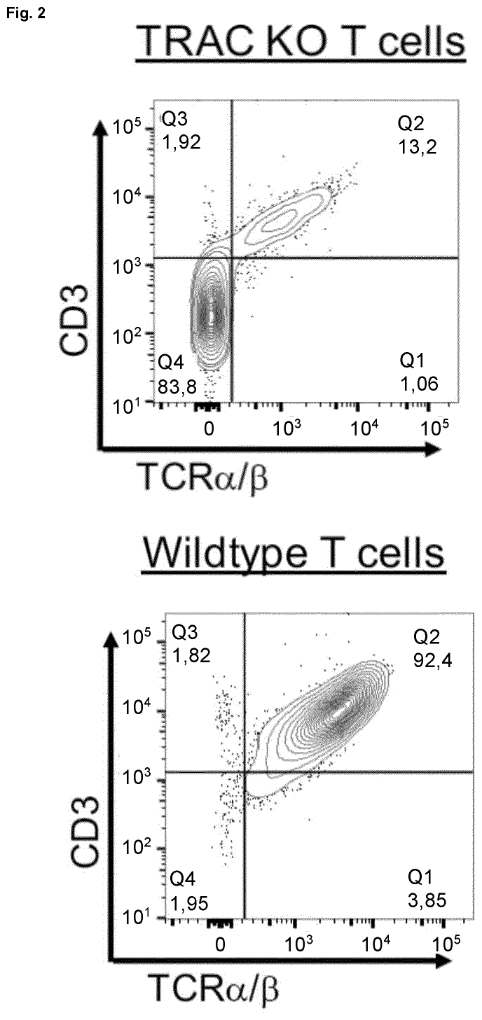

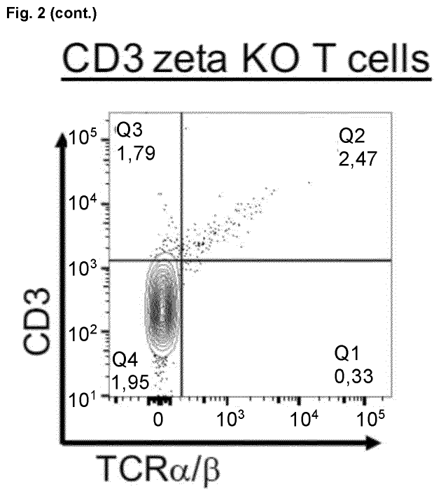

FIG. 2: Knock-out of TRAC or CD247 prevent formation of TCR-CD3 complex on the surface of primary human T cells

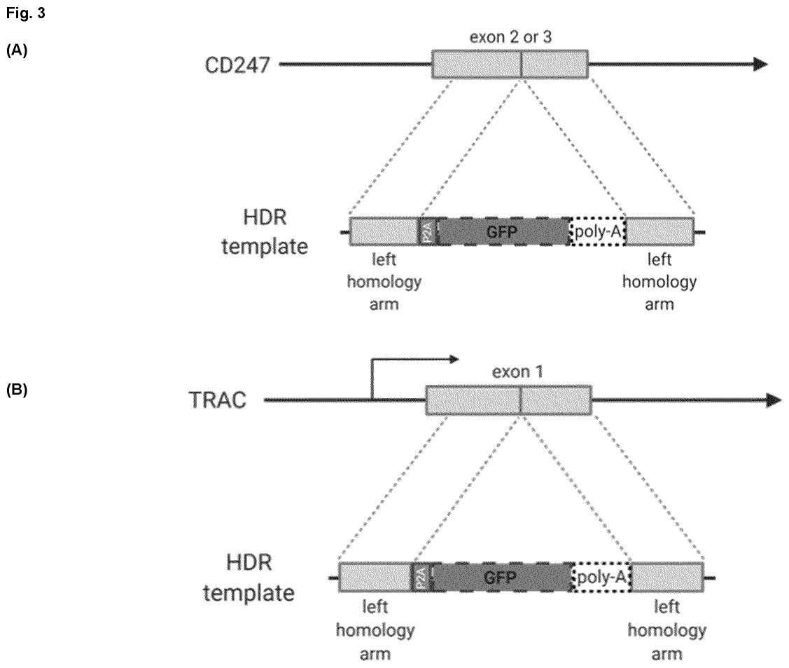

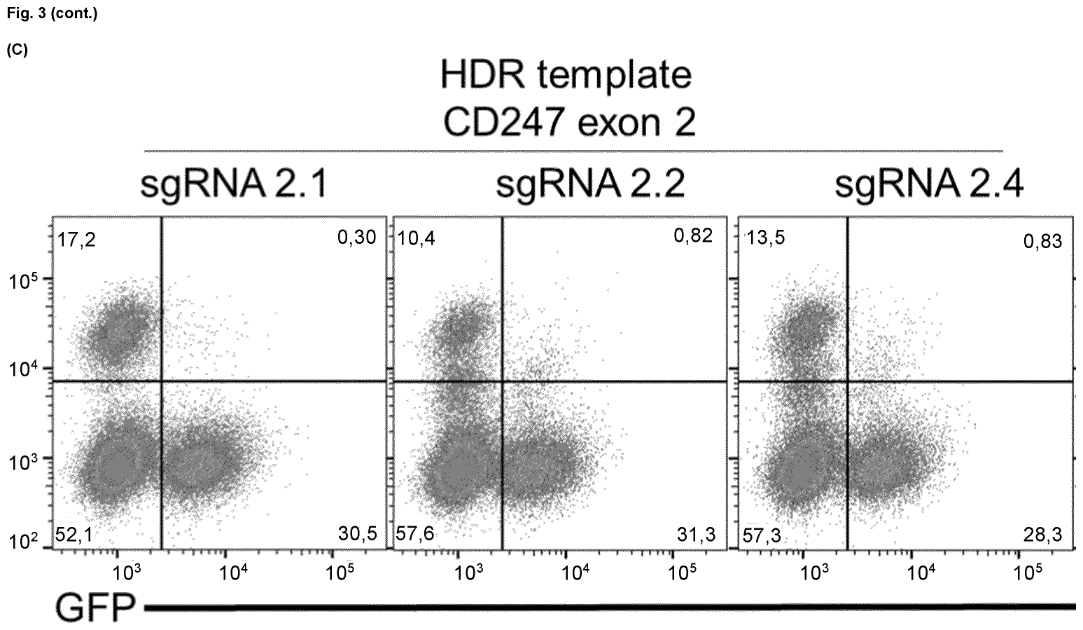

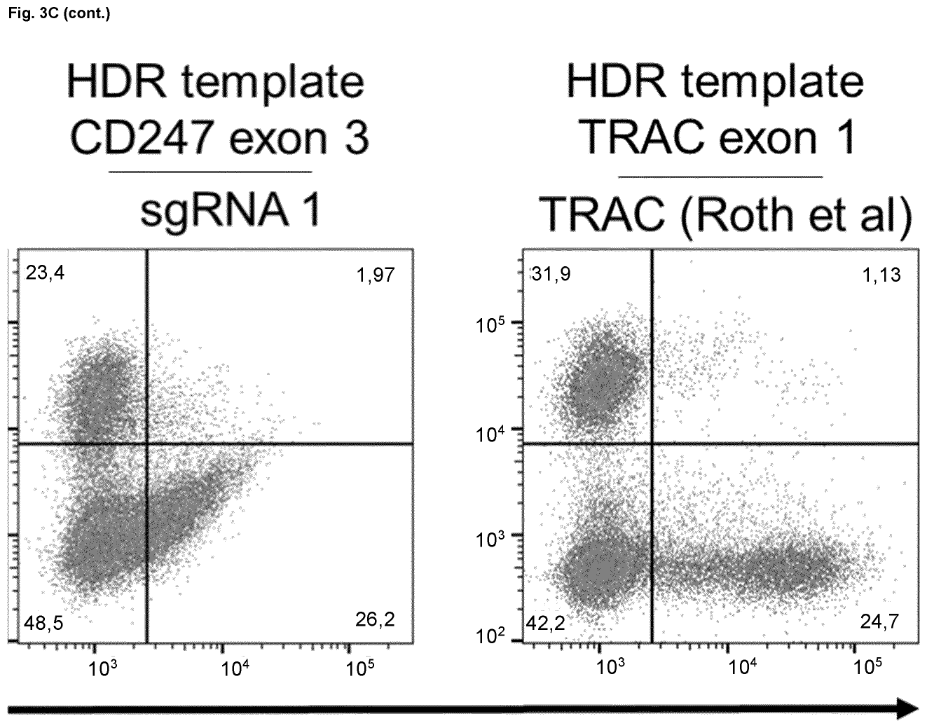

FIG. 3: In-frame insertion of GFP into different T cell receptor complex genes leads to loss of CD3-TCR complex surface expression

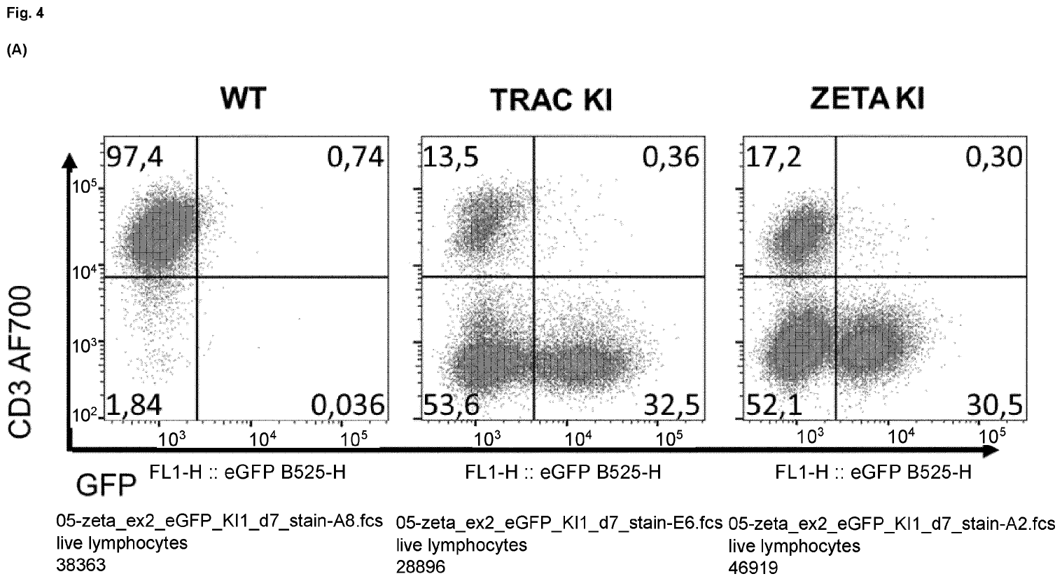

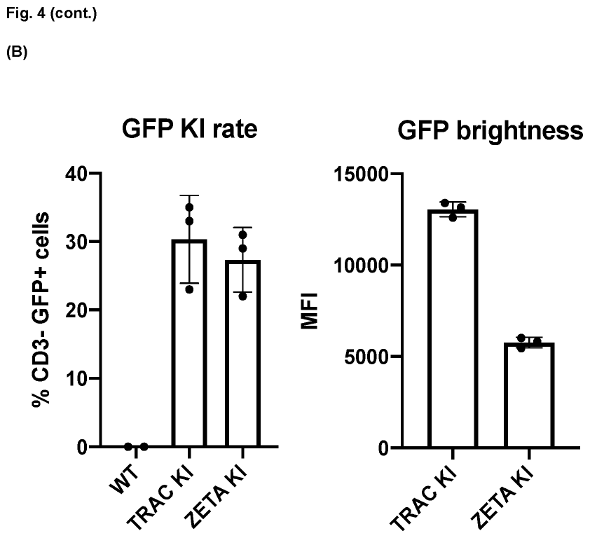

FIG. 4: GFP intensity is lower when expressed under control of the CD247 promotor compared to the TRAC promotor

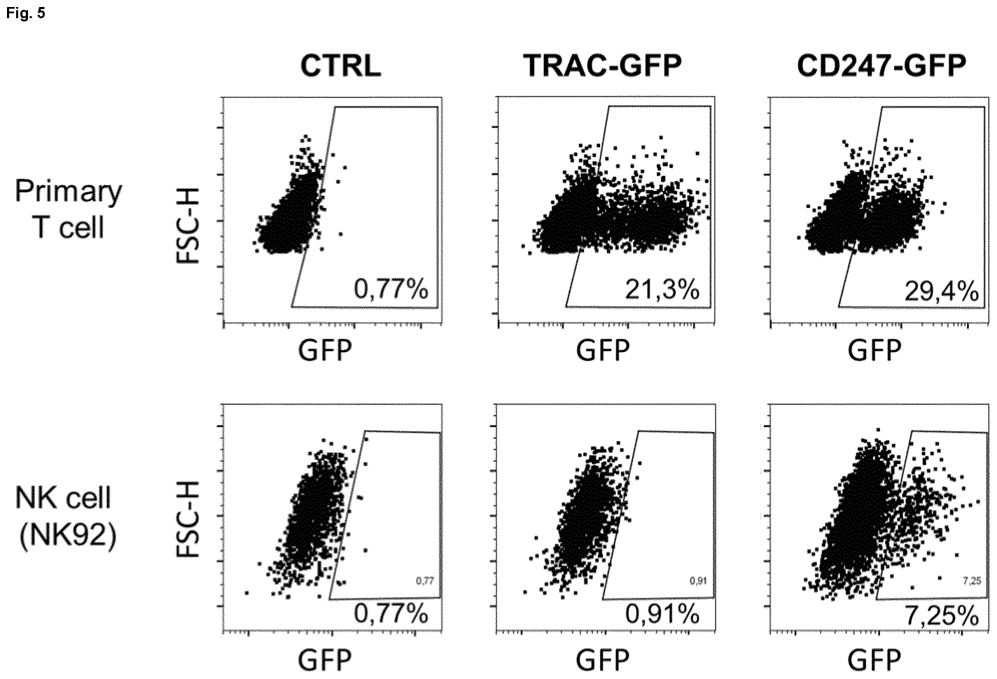

FIG. 5: In NK cells, transgenes can be expressed under control of CD247 but not TRAC

FIG. 6: Insertion of complete and zeta-deficient CAR transgenes into CD247 gene induce CAR protein surface expression

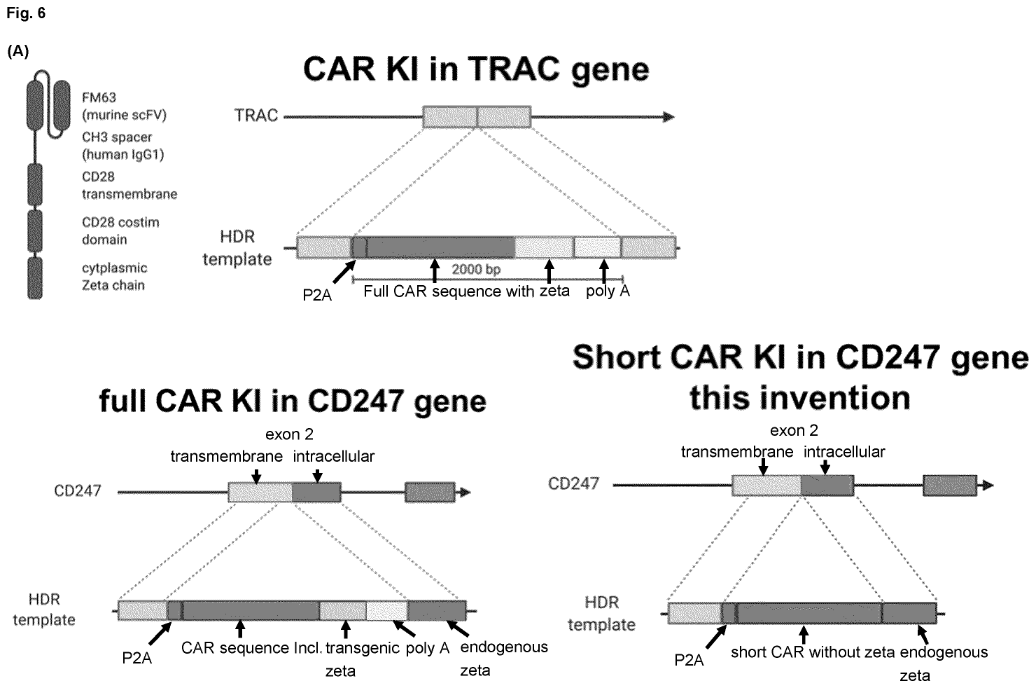

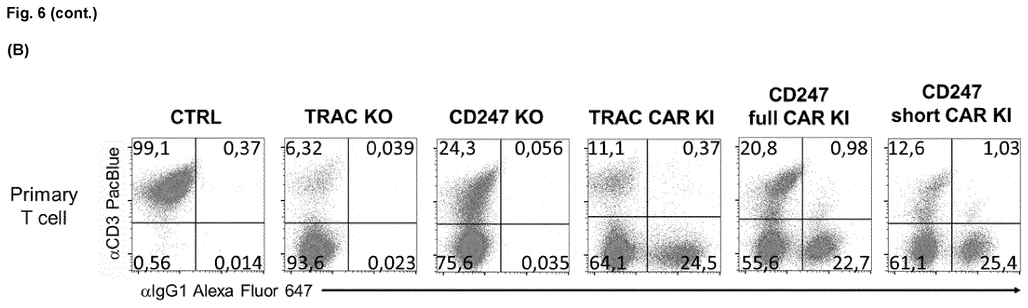

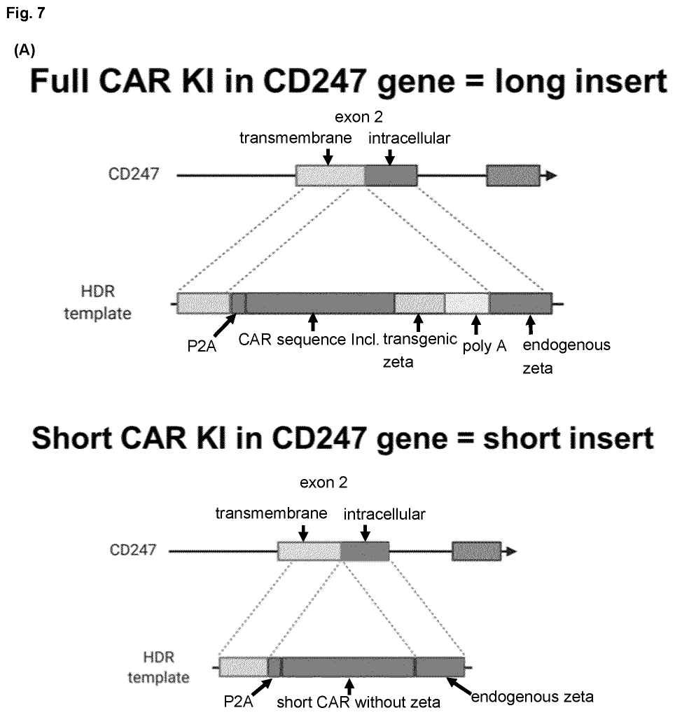

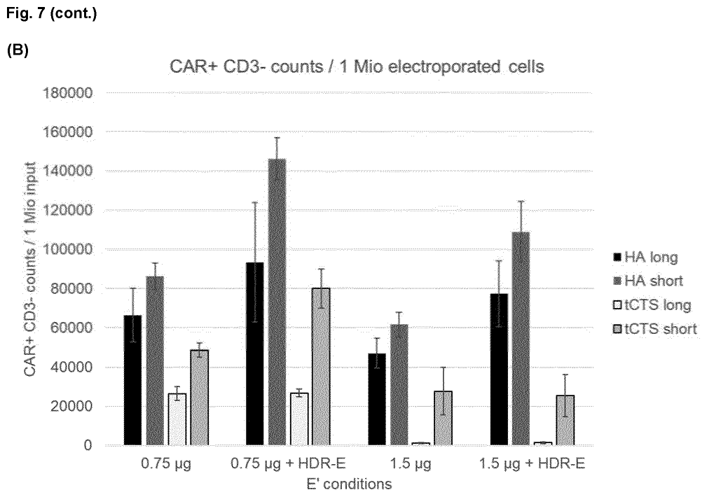

FIG. 7: Introducing shorter insert size tends to increase absolute CAR T cell generation after non-viral gene insertion

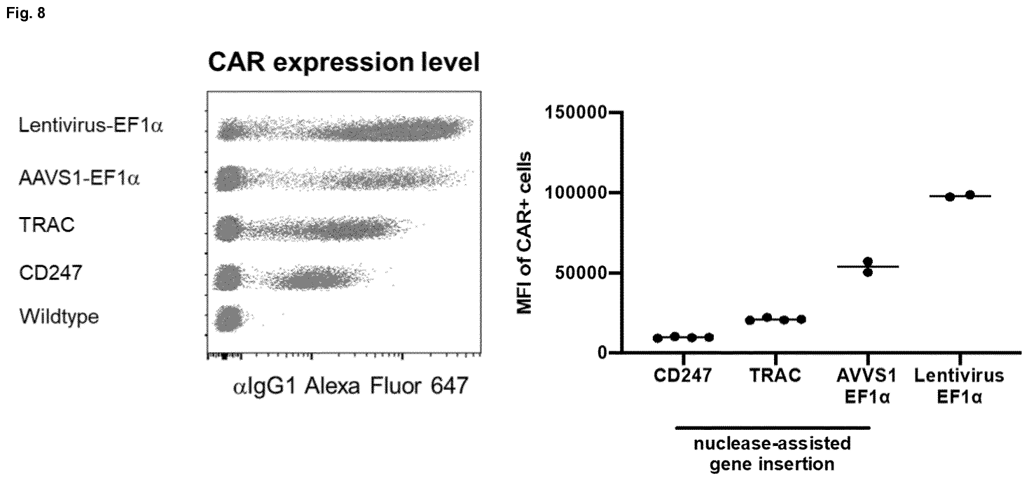

FIG. 8: CAR expression level is regulated most strictly under control of the CD247 gene

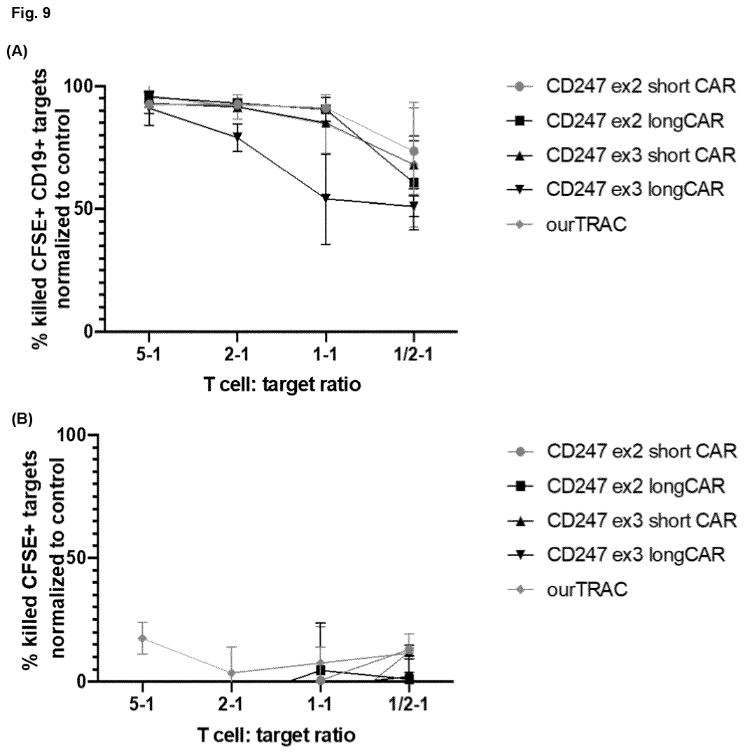

FIG. 9: Short CAR CD247 ki CAR T cell lyse CD19+ target cells in antigen-specific and dose-dependent fashion (as well as at a comparable rate to TRAC/CD247-integrated full CARs)

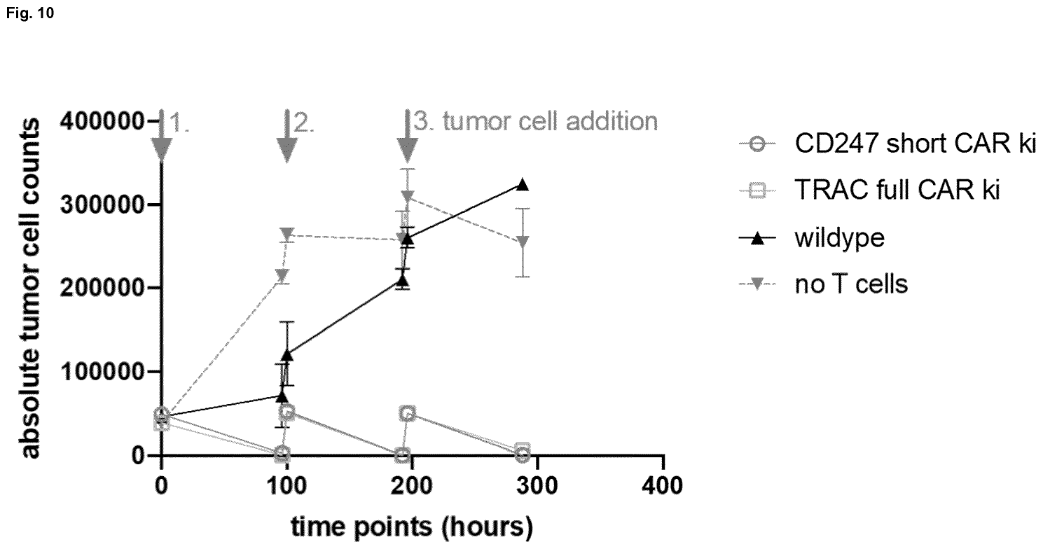

FIG. 10: CD247 short CAR KI T cells perform comparable to full CAR knock-in in serial tumor challenge

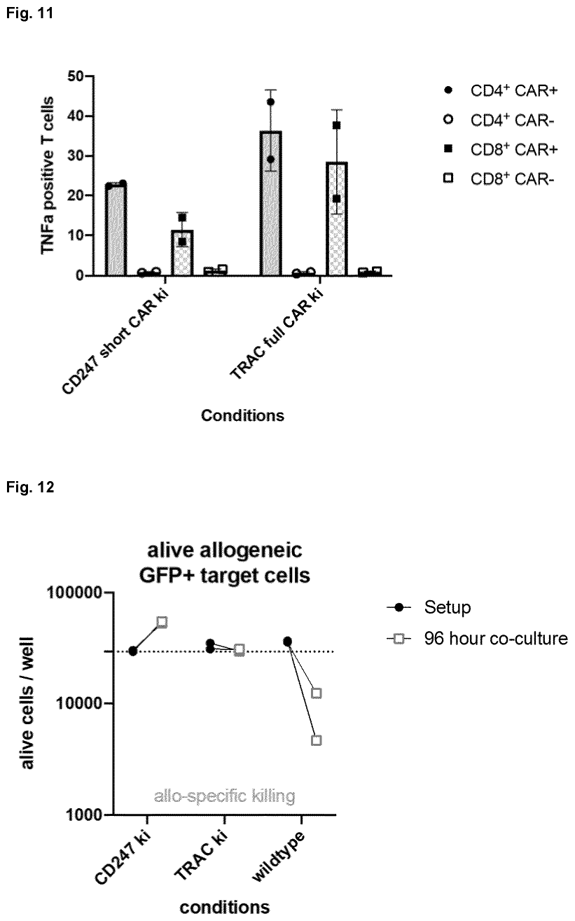

FIG. 11: Reduced cytokine production after antigen-engagement by truncated CARs integrated in the CD247 gene vs full CAR in the TRAC locus

FIG. 12: CD247 KI abolishes allo-reactivity of CAR T comparable to TRAC KI

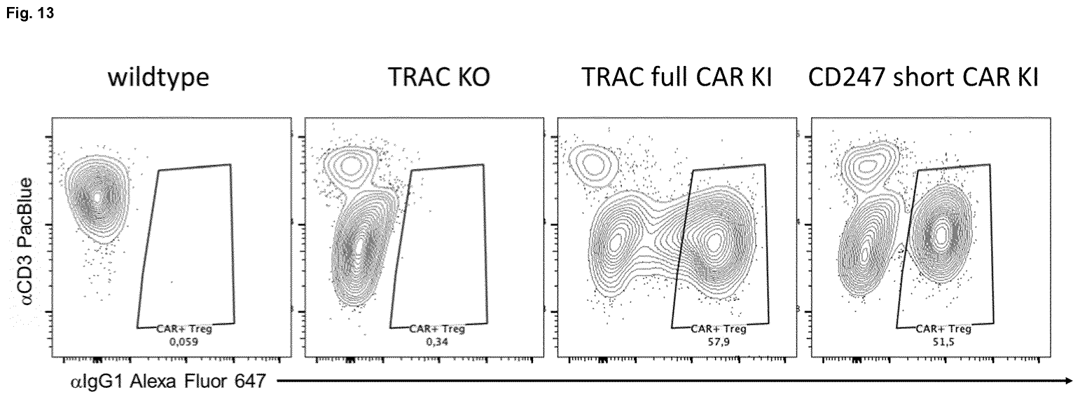

FIG. 13: Integration of truncated CARs into CD247 gene can be used to generate CAR+ regulatory T cells

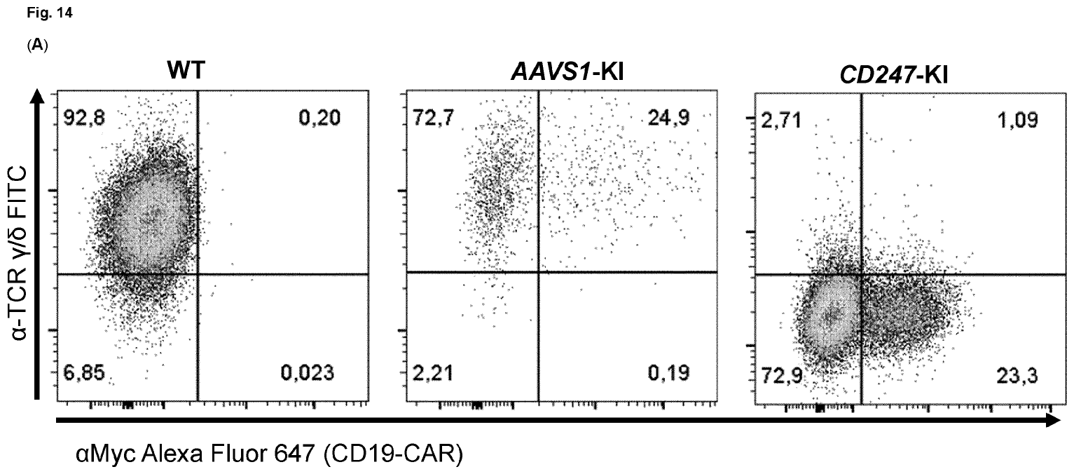

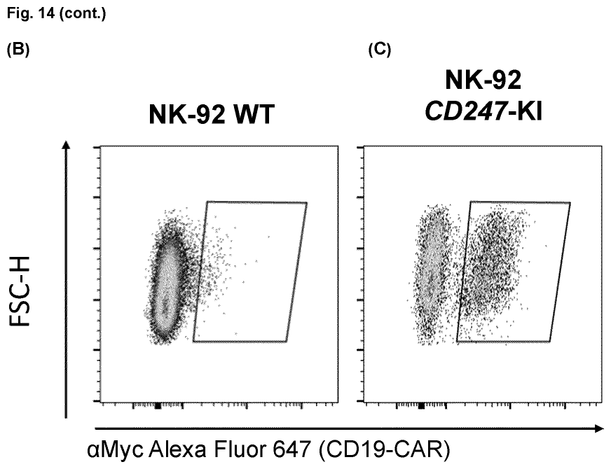

FIG. 14: TCR γ/δ T cells and NK cell can be reprogrammed by CD247-KI approach

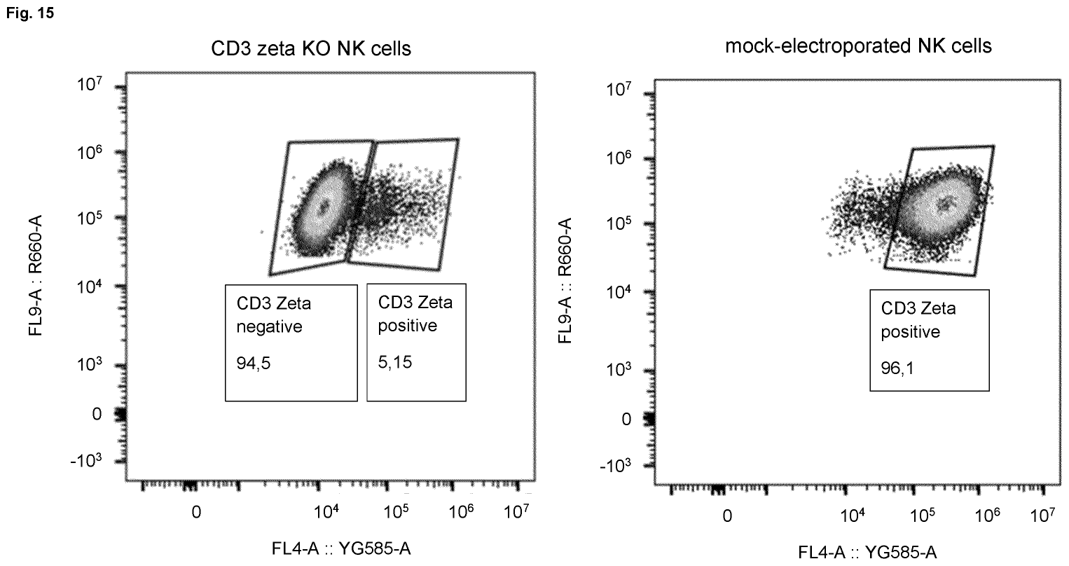

FIG. 15: CD3 zeta protein is expressed in NK cells

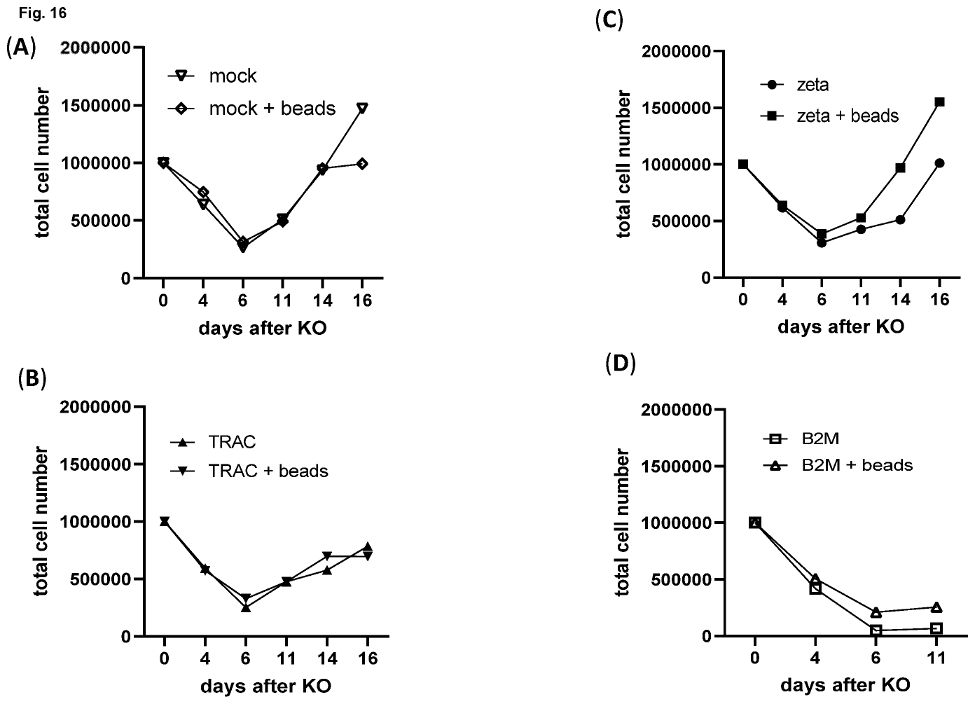

FIG. 16: Primary NK cells expand after CD247 knockout

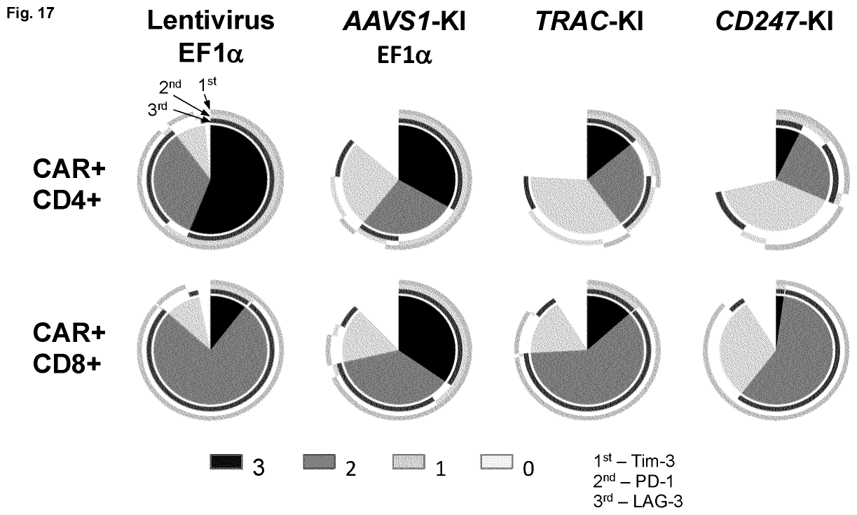

FIG. 17: Expression of surface proteins associated with T cell exhaustion were compared after serial tumor challenge assay.

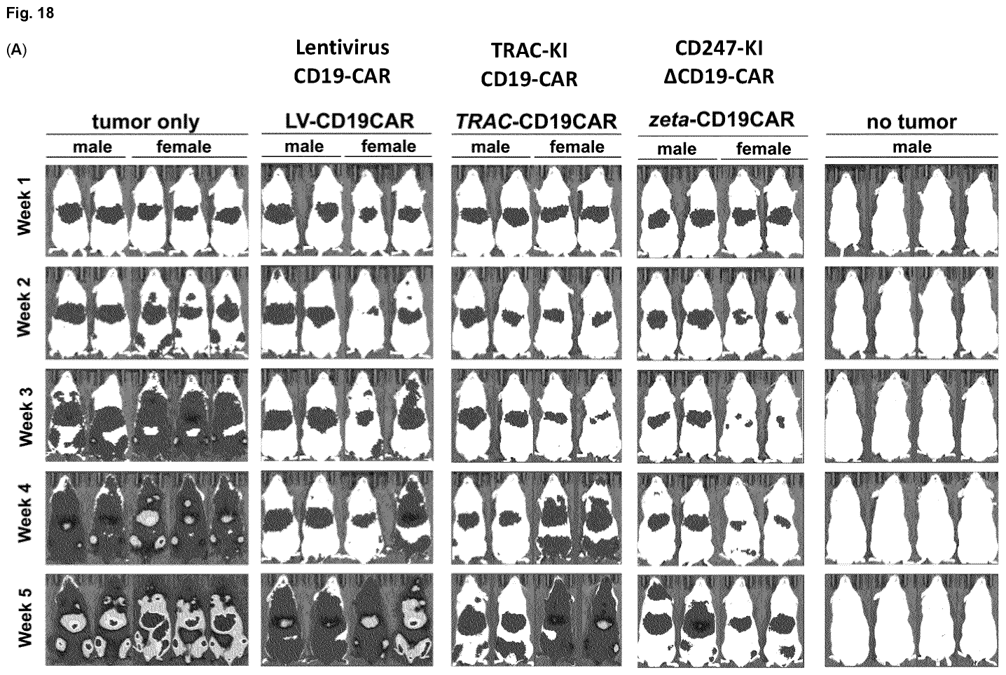

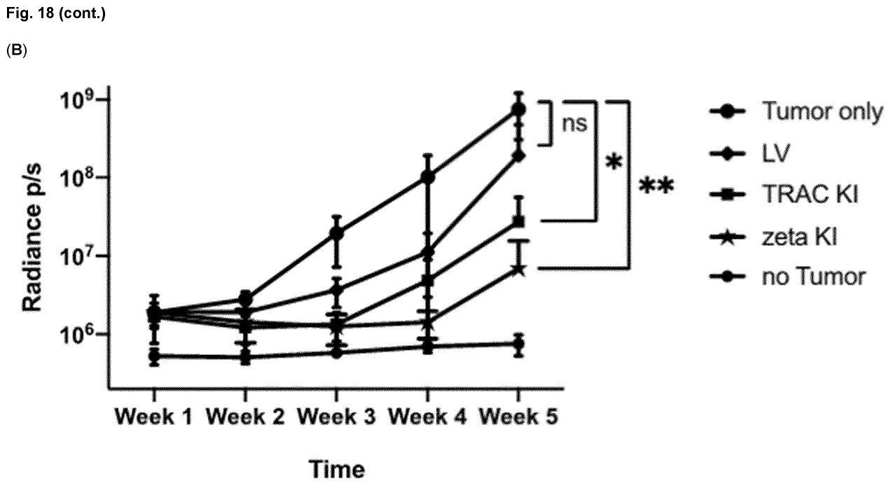

FIG. 18: Superior leukemia control by CD247-KI CD19-CAR T cells

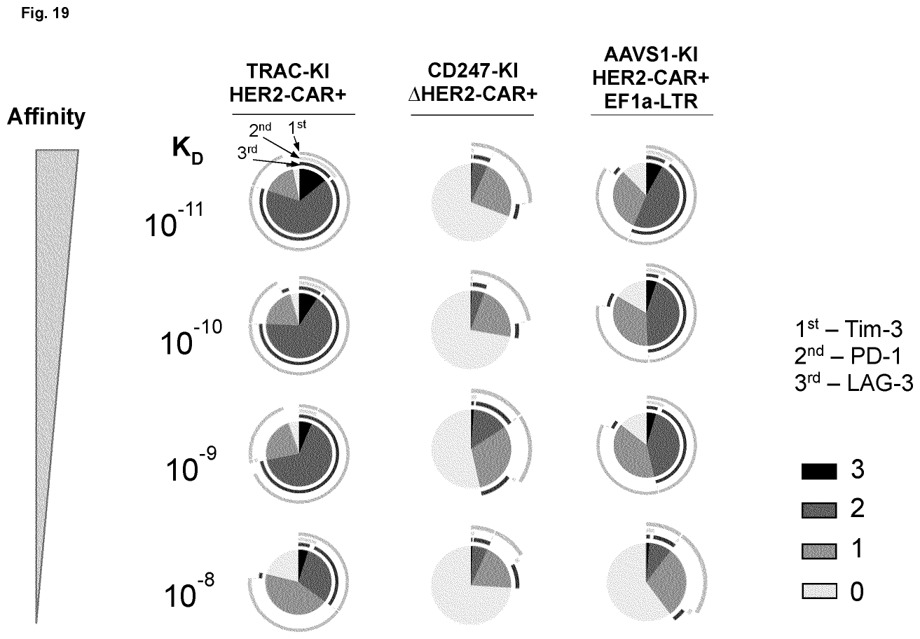

FIG. 19: CD247-KI HER2-CAR T display reduced exhaustion marker expression after in vitro expansion (d14)

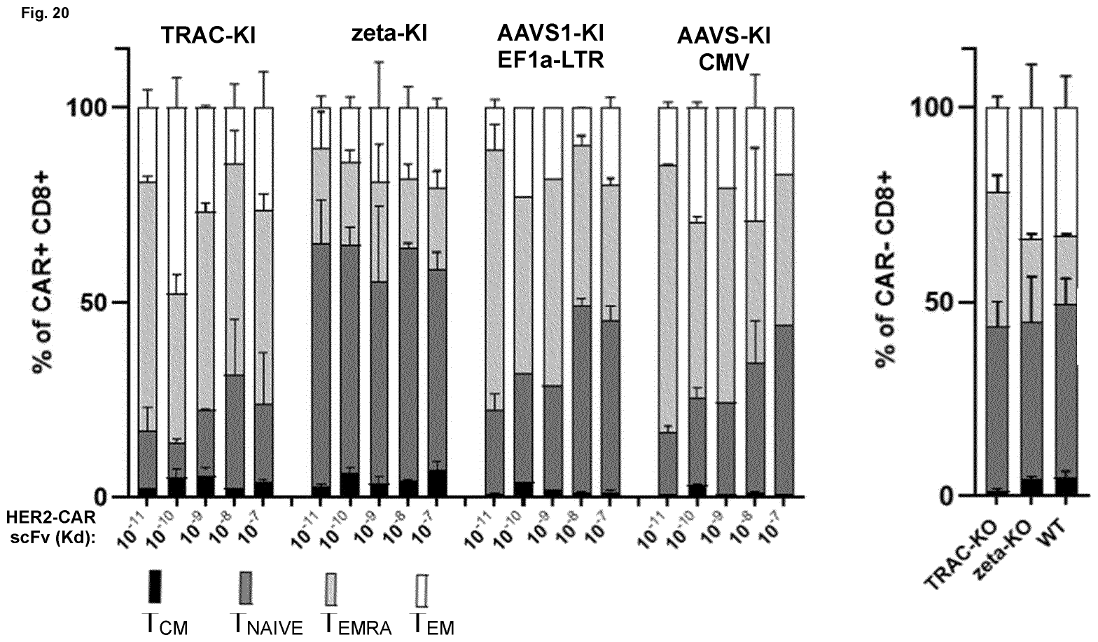

FIG. 20: Reduced differentiation of CD247-KI HER2-CAR T cells after 2 week in vitro expansion.

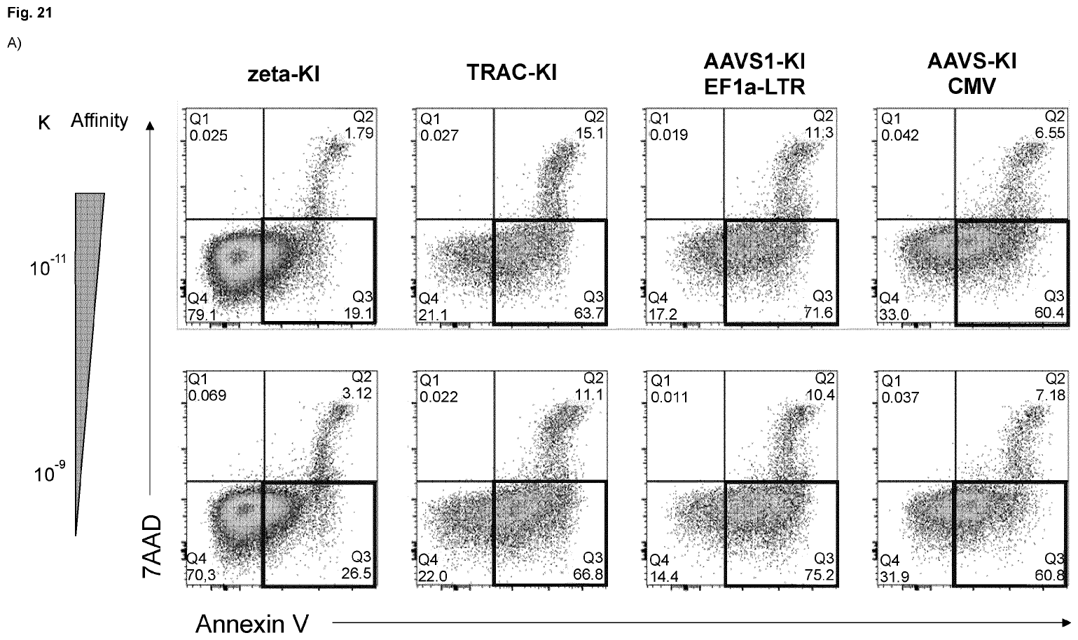

FIG. 21: Reduced activation-induced cell death for CD247-KI Her2/neu-CARs after stimulation

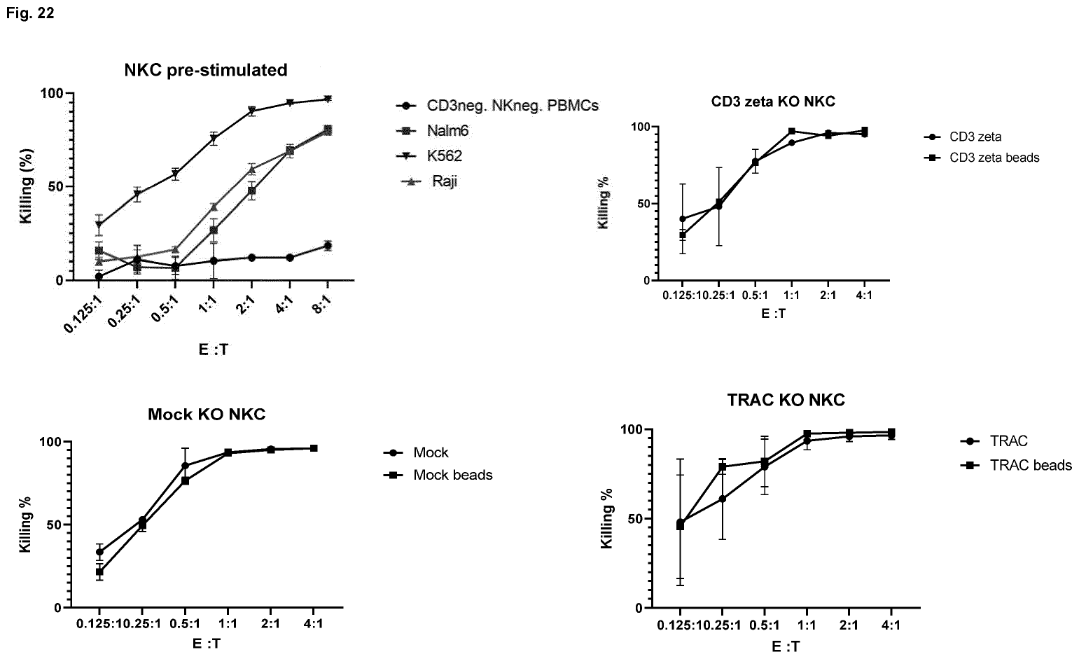

FIG. 22: Intact killing capacity of primary NK cells (NKC) with CD3 zeta KO

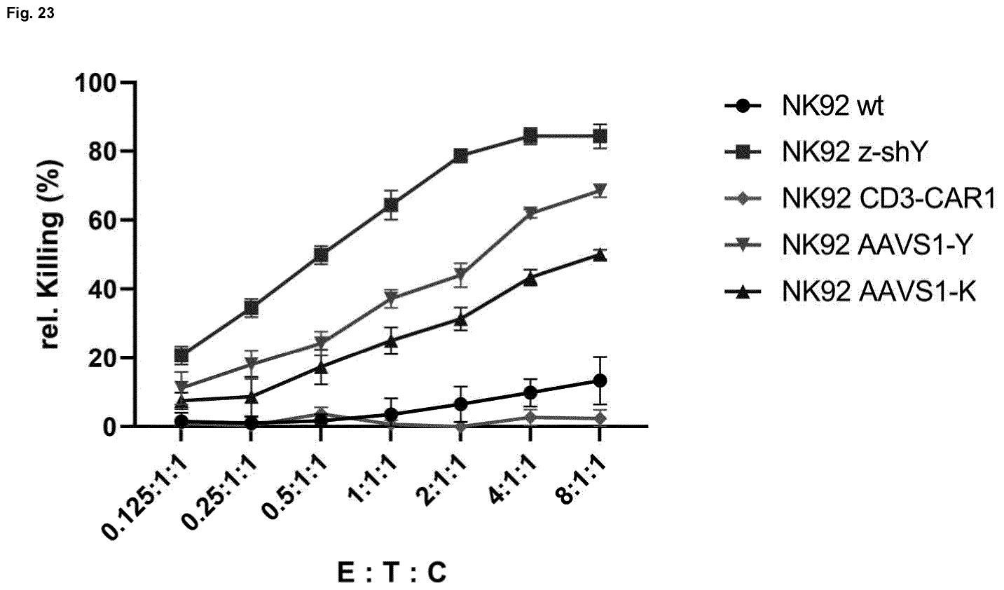

FIG. 23: Zeta-KI CAR-NK cells have superior killing capacity than CAR-NK that overexpress the CAR from an ectopic locus (hAAVS1)

### DETAILED DESCRIPTION OF THE FIGURES

FIG. 1: New strategy fuses zeta-deficient CAR transgenes to endogenous CD3 zeta on genetic level: (A) CD3-zeta protein expression form the CD247 gene. In the process of RNA transcription, CD247 exons are spliced together to form a mature CD247 messenger RNA, which serves as a template for protein translation by the ribosome. The formed CD3-zeta protein assembles with other proteins to form a fully functioning TCR/CD3 complex. The intracellular domain of the CD3-zeta protein within the TCR/CD3 complex is identical to the CD3-zeta domain of conventional Chimeric Antigen Receptors. (B) The sketch also illustrates this invention. A truncated, CD3-zeta deficient CAR transgene (preferably but not exclusively encoding a self-cleaving 2A-peptide, an antigen binding domain (e.g. an scFv), a hinge/spacer domain, a transmembrane domain and one or more co-stimulatory or regulatory domains but no Immunoreceptor Tyrosine-based Activation Motif (ITAM) containing signaling domain) is inserted into the CD247 gene. The modified CD247 gene is transcribed into mRNA encoding a full CAR with an endogenous CD3-zeta domain. During protein translation, the N-terminus of the expressed fusion protein is cleaved off the CAR protein via self-cleavage of the 2A peptide. While a functional CAR Protein is expressed on the surface of edited immune cells, the cleaved N-terminus of the CD3-zeta gene is non-functional and cannot serve for TCR/CD3 complex formation. As a result, successfully edited immune cells can express a CAR protein but are unable to express a TCR.

FIG. 2: Knock-out of TRAC or CD247 prevent formation of TCR-CD3 complex on the surface of primary human T cells: Healthy human T cells were isolated from venous blood and polyclonally stimulated on anti-CD3−/anti-CD28-antibody coated tissue culture plates in the presence of IL-7 and IL-15 (each at 10 ng/ml). Two days after activation, T cells were electroporated with sgRNAs targeting either the constant region of the T cell receptor alpha chain gene (TRAC, left panel) or the CD247 gene (CD3-zeta, right panel). After four days of subsequent expansion in cytokine containing medium, cells were analyzed for CD3 and TCRoc/p expression via flow cytometry. [KI=knock-in; KO=knock-out]

FIG. 3: In-frame insertion of GFP into different T cell receptor complex genes leads to loss of CD3-TCR complex surface expression: In this experiment, nuclease-assisted gene insertion of green fluorescent protein (GFP) reporter transgenes into the protein-coding reading frame of either the CD247 gene (encoding for CD3 zeta) or the TRAC gene (encoding for TCR alpha chain) was performed to investigate the effect on CD3-TCR surface expression. As indicated by flow cytometric protein analysis, most GFP expression was observed in the CD3-TCR negative population (Y-axis). Multiple Cas9 nucleases with different sgRNAs were used for CD247 exon 2 and 3 achieving similar results. Insertion of GFP into TRAC gene mirrored results seen in CD247, despite higher maximum GFP intensity.

FIG. 4: GFP intensity is lower when expressed under control of the CD247 promoter compared to the TRAC promoter: (A) This subanalysis from the previous experiment (FIG. 3) compares GFP knock-in (KI) rates and fluorescence intensity in three replicates summarized for the preferred conditions (CD247: HDR template exon 2+ sgRNA 2.1; TRAC: exon 1 Charité+TRAC sg1). (B) While relative KI did not differ significantly between TRAC and CD247, GFP expressed under control of the CD247 promotor leads to a lower median fluorescence intensity (MFI). [KI=knock-in; KO=knock-out]

FIG. 5: In NK cells, transgenes can be expressed under control of CD247 but not TRAC: For this experiment, nuclease assisted gene insertion was repeated in Natural Killer (NK) cells (modeled by NK92 cell line) using preferred HDR templates and sgRNA-nuclease as before (FIG. 6, CD247: HDR template exon 2+ sgRNA 2.1; TRAC: exon 1 Charité+TRAC sg 1). To this end, NK92 cells were expanded in RPM11640 medium supplemented with 10% FCS, 1% penicillin/streptomycin (Biochrom) and IL-2 (500 IU/ml). Four days prior to electroporation, NK92 were seeded in antibiotic free medium. In preparation, RNPs and HDRTs as well as cells were prepared as previously described (FIG. 3). Nucleofection was performed with program CA137. NK92 cells were subsequently cultured for 2 days. Flow cytometric analysis was performed as before, but without CD3 antibody. Here, representative flow cytometric analysis are shown and compared to results from experiments with primary T cells (FIG. 4).

FIG. 6: Insertion of complete and CD3 zeta-deficient CAR transgenes into CD247 gene induce CAR protein surface expression: Nuclease assisted gene insertion was used to introduce complete or CD3 zeta-deficient (truncated) CAR transgenes into the CD247 gene. To perform this experiment, new HDR templates were designed. We rationally designed a 2nd generation CAR consisting of a murine CD19-specific single chain variable fragment (scFv), clone FMC63, an intermediate hinge CH3 spacer (derived from human Fc-IgG1), a CD28 transmembrane domain, a CD28 costimulatory intracellular domain and the CD3 cytoplasmic zeta chain. This CAR was cloned into HDR templates presented before (FIG. 3-5, Table 1 in Example 5). For CD247 HDR templates, a full CAR expression module with CD3 zeta and poly-A terminator sequence as well as a short CAR sequence lacking a poly-A terminator sequence were generated to test the hypothesis of this invention. Nucleofection was performed as described in FIG. 3. Flow cytometry was performed 7 days after nucleofection. To stain for CAR via its IgG1 hinge, cells were stained with LIVE/dead and anti-Fc Alexa Fluor 647 (Jackson) first. After washing, a second surface stain with anti-CD3 PacBlue (Biolegend) was done. (Note: forthis experiment CD247 sg2.2 was used. For all subsequent experiment CD247 sg2.1 is used)

FIG. 7: Introducing shorter insert size tends to increase absolute CAR T cell generation after non-viral gene insertion: The effect of insert size (full CAR vs short zeta-less CARs) on absolute CAR+ T cell counts was compared after nuclease assisted gene insertion with two different non-viral dsDNA HDR template variants. HDR templates were either generated with regular homology arms (HA) (FIG. 3, previously described in PMID: 29995861) or with homology arms modified with truncated Cas9 targeting sequences (tCTS) (16 bp, previously described in PMID: 31819258). After Electroporation, cells were either cultured in T cell medium or in T cell medium supplemented with 15 uM (0.5%) Alt-R HDR-Enhancer (Integrated DNA Technologies, Catalog #1081073). Two HDR template concentrations (0.75 or 1.50 ug/20 ul Electroporation) were compared. Regardless of the HDR template format used, smaller insert size due to the zeta-less CAR transgenes lead to improved CAR+ T cell recovery 7 days after transfection, thereby indicating an advantage for CAR T cell generation using this approach.

FIG. 8: CAR expression level is regulated most strictly under control of the CD247 gene: In this experiment, CAR expression levels were compared between CAR T cells generated by different strategies. Representative flow cytometry plots and summary of median fluorescence intensity of CAR+CD3− cells are shown. CAR T cells generated through insertion of truncated CAR transgenes into the CD247 exon 2 (sg 2.1) display the lowest MFI compared to TRAC-integrated CARs or other controls. For overexpression controls, the CAR presented in FIG. 6 was cloned into an expression cassette commonly used for generation of CAR T cells via lentiviruses or via transposase-transposon technology. It includes an EF1-alpha promoter and a BGH poly-A terminator sequence. The expression cassette was cloned into an HDR template for insertion into the human safe harbor locus hAAVS1. Additionally, the CAR was cloned into the SIN epHIV7 lentiviral vector plasmid for virus preparation and lentiviral transduction was performed as previously described (PMID: 22707919).

FIG. 9: Short CAR CD247 KI CART cell lyse CD19+ target cells in antigen-specific and dose-dependent fashion (as well as at a comparable rate to TRAC/CD247-integrated full CARs): CAR T cells generated either by short (truncated) or full CAR transgenes lysed target cells in an antigen- and dose-dependent fashion and with similar efficacy as TRAC-integrated CAR T cells. The only CAR T cell condition with reduced killing capacity at 2-1 and 1-1 T cell: target ratios was generated with the HDR template CD247 exon 3 inserting a full CAR expression cassette. To test the cytolytic activity of CAR T cells generated through transgene insertion into the CD247 locus, overnight co-culture assays with tumor cells were performed. CAR T cells generated either with nuclease assisted gene insertion into the CD247 exon 2, CD247 exon 3 or TRAC exon 1 were tested. (A) CD19+ tumor cells (Nalm6) or (B) CD19− tumor cells (Jurkat) were stained with CFSE to allow discrimination between targets (tumor cells) and effectors (CAR T cells). Co-cultures were performed in 96-U-Bottom-Well plates with 25.000 tumor cells per well. Depending on desired T cell: target ratio, respective numbers of CAR T cells were added to the specific wells. As controls, tumor cells were cultured without any T cells. On the following day, the cells were washed with PBS, centrifuged at 400 G for 10 minutes and stained with LIVE/Dead dye to exlude apoptotic cells. Subsequently, surviving cells were quantified by volume-based flow cytometric acquisition (Cytoflex LX, Beckman). The percentage of killing was calculated by normalizing to the negative control. TRAC-integrated CAR T cells served as a positive control. [KI=knock-in]

FIG. 10: CD247 short CAR KI T cells perform comparable to full CAR KI in serial tumor challenge: CAR T cells reprogrammed by insertion of truncated (zeta-deficient) CARs into the CD247 gene were able to eliminate tumor cells repeatedly. At the day of assay setup, 25.000 T cells were mixed with 50.000 CD19+ tumor cells (Nalm6) in cytokine-free medium (RPMI1640+10% FCS). Per condition, four wells were prepared. The first well was analysed at setup day to control absolute tumor cell counts. Every third day, one well per condition was analysed and 50.000 CD19+ tumor cells (Nalm6) are added to the remaining wells. In summary, absolute tumor cell counts are presented. In negative controls (conditions with no T cells or with wildtype T cells), tumor cells grew uncontrolled. Both TRAC-integrated full-CAR equipped and CD247-integrated short CAR-reprogrammed T cells repeatedly eliminated tumor cells. [KI=knock-in]

FIG. 11: Reduced cytokine production after antigen-engagement by truncated CARs integrated in the CD247 gene vs full CAR in the TRAC locus: As presented before (e.g. FIG. 8), CD247 integrated zeta-deficient CAR-reprogrammed T cells have a lower CAR expression level.

While cytolytic activity remained unaffected by lower CAR surface density (FIG. 9,10), secretion of inflammatory cytokine TNF-alpha was reduced after challenge with CD19+ tumor cells (Nalm6) in both CD4+ and CD8+ T cells. One hour after start of co-culture, brefeldin A (Sigma) was added at 10 μg/ml to prevent cytokine secretion into the medium. After 6 hour total incubation, the reaction was stopped by washing in cold PBS. Then, surface staining with LIVE/dead Blue Cell Dye (1:100, Thermo Fisher) was performed to exclude dead cells. Fixation and permeabilization was performed with Foxp3 Transcription Staining Kit according to the manufacturer's recommendations (Ebioscience, ThermoFisher). Subsequent intracellular staining was performed in two steps; first anti-Fc Alexa Fluor 647 (1:50, Jackson) was stained at 4° C. for 20 minutes. After wash with Permbuffer (same kit as before), a second intracellular staining was performed with antibodies for CD4, CD8 and TNF-alpha.

FIG. 12: CD247 KI abolishes allo-reactivity of CAR T comparable to TRAC KI: As presented before (e.g. FIG. 6), insertion of (truncated) CARs into the TRAC as well as the CD247 gene eliminates the CD3-TCR from the T cell surface. To test whether this eliminates allogeneic cell killing, we performed co-culture assay with human T cells from two unmatched healthy donors with strong allo-reactivity against the CD19-negative cell line MM1S expressing a GFP reporter. After four days, unmodified wildtype T cells eliminated a significant fraction of the tumor cells. In contrast the amount of tumor cells remained unchanged for TRAC-inserted CAR T cells or they increased in the presence of the CD247-integrated CAR T cells, indicating undectable rate of allo-activity.

FIG. 13: Integration of truncated CARs into CD247 gene can be used to generate CAR+ regulatory T cells: CD4+ Treg were generated from PBMCs as described before (FIG. 3). On the day of PBMC isolation, CD8+ cells were eliminated using CD8+ microbead depletion (Miltenyi Biotec). The following day, CD25+ cells were enriched (CD25 microbeads, Miltenyi Biotec) from the CD8-negative cell fraction to isolate CD4+CD25+ T cells which contain >70% regulatory T cells. Subsequently, the cells activated with anti-CD3/28-coated beads and expanded in X-Vivo cell medium (Lonza) supplemented with 10% FCS, high doses of IL-2 (500 IU/ml) and Rapamycin (Treg medium) as described before (PMID: 33087345). On day 7 after PBMC isolation, regulatory T cells were re-stimulated with anti-CD3/28-coated beads. Next day, the beads were magnetically depleted and nucleofected as described before (FIG. 3). Rescue was performed with the Treg medium. After resting one more day, the nucleofected T cells were re-stimulated with irradiated CD19+ target cells (Nalm-6) and sub-cultured for additional 7 days prior to flow cytometric analysis.

FIG. 14: TCR γ/δ T cells and NK cell can be reprogrammed by CD247-KI approach. (A) Positive enrichment for TCR γ/δ was performed using Miltenyi MACS column purification. Subsequently, cells were stimulated on plate-bound anti-CD3/CD28 and nucleofected with either CRISPR-Cas9 ribonucleoproteins and HDR donor templates for AAVS1 or CD247. Representative flow cytometry readout shows CAR expression in efficient editing of CD247 locus and CAR expression 4 days after electroporation. (n=2 biological replicates) (B) NK cells could be reprogrammed by CD247-KI approach. As a model for primary NK cells, the NK92 cell line was transfected with CRISPR-Cas9 ribonucleoproteins and dsDNA donors. While TRAC-KI does not induce GFP/CAR expression, CD247 gene targeting allows expression of transgenes. (C) CD247-KI CAR enhances killing of Nalm-6 CD19+, but not CD19-negative control cells in a 6 hour VITAL assay (n=1).

FIG. 15: CD3 zeta protein is expressed in NK cells

Unlike previously stated, primary NK cells (NKC) do express the CD3 zeta protein. Primary NK cells were isolated from PBMC via the human NK cell isolation kit via MACS according to the manufacturer's instructions (Miltenyi Biotec) exceeding 95% purity indicated by flow cytometric analysis of CD3-negative CD56-positive NK cell phenotype. Subsequently, NK cells were expanded in MACS NK cell expansion medium (Miltenyi Biotec) supplemented with 10% FCS and IL-2 as well as IL-15. Proliferating NK cells were harvested, washed and 1×10e6 NK cells were resuspended in 20 I P3 buffer (Lonza) and electroporated with CD247-specific RNP (incl. sgRNA 2.1) via Lonza 4D Nucleofector's program DN-100. For flow cytometric analysis, NK cells were labled with Live/Dead Blue Fixable Dye (ThermoFisher), then fixed, permeabilised and stained with anti-CD247 PE antibody and anti-CD56 AlexaFluor 647 antibody (both Biolegend).

FIG. 16: Primary NK cells expand after CD3 zeta KO. Primary NK cells expand with or without beads from human NK cell activation/expansion kit (Miltenyi) after nucleofection (A) without RNP (mock), (B) TRAC-specific RNP (TRAC KO, TRAC sg 1 (SEQ ID NO 55)), (C) CD247-specific RNP (zeta KO, CD247 sg2.1 (SEQ ID NO 52)) or (D) B2M-specific RNP (B2M KO, sgRNA target sequence: SEQ ID NO 65). Cell counts were determined by trypan blue exclusion technique and Neubauer counting chamber. NK cells were able to proliferate after genetic deletion of CD247/CD3 zeta (Flow cytometry confirmed >90% KO, FIG. 15). However B2M disruption inflicted severe fratricide due to loss of HLA-class 1 expression and consequently missing-self recognition. Results from representative donor (n=3). [RNP: ribonucleoprotein complex, consisting of a sgRNA and recombinant Sp.Cas9 protein]

FIG. 17: Expression of surface proteins associated with T cell exhaustion were compared after serial tumor challenge assay. Plots show representative SPICE analysis of flow cytometry readout (n=2 biological replicates). CAR T cells from A) were expanded for 14 days total and cryopreserved for 5 months. Prior to first tumor challenge, CAR T cells were thawed and rested overnight. Then, CAR+ T cells were counted and co-cultured with irradiated CD19+ GFP+ Nalm-6 tumor cells at 1 tumor: 2 CAR+ T cell ratio in medium containing 50 IU/ml IL-2. Every 12 hours new irradiated Nalm-6 cells were added to the respective wells. Flow cytometry was performed 24 hours after last tumor challenge.

FIG. 18: Superior leukemia control by CD247-KI CD19-CAR T cells. Comparison of the therapeutic efficacies between CD247-KI, TRAC-KI and lentivirally-transduced CD19-CAR T cells in a pre-B acute lymphoblastic leukemia xenograft mouse model. Experimental set-up: Eight-weeks old Nod.Rag.Gammma (NRG) mice were challenged i.v. with 5×10⁵ Nalm-6/GFP/fLuc cells. Mice not receiving leukemia (‘no tumor’) were used as controls for analyses. 4 days after leukemia challenge, mice were randomized among three cohorts: (i) injected i.v. with phosphate-buffered saline (PBS, corresponding to the ‘tumor only’ group), (ii) injected i.v. with 5×10⁵ CD19-CAR T cells generated via lentiviral transfer and consecutive TRAC KO (‘LV-CD19CAR’), (iii) injected i.v. with 5×10⁵ CD19-specific TCR-deficient CAR T cells generated by TRAC-knockin with a complete CAR (‘TRAC-KI CD19-CAR’) or (iii) CD247-knockin of a short/truncated CD19-CAR (Zeta-KI CD19-CAR). The residual CD3+ T cells were depleted from both types of CAR T cell types prior to infusion. Mice were monitored for disease severity every 2-3 days and body weights were measured weekly. Leukemia engraftment, bio-distribution and progression was assessed by weekly bioluminescence imaging (BLI) analyses. The experimental endpoint was five weeks after leukemia challenge. (A) BLI of all mice over a 5 week observation period. BLI pictures generated sequentially from weeks 1 to 5. Frontal pictures of mice are shown, with bioluminescence signal radiance (photons/sec/cm²/sr) depicted by the color barcode on the right side. (B) Quantification of leukemia spread by BLI. Radiance (photons/sec) depicts each pixel summed over the regions of interest (ROI) area containing the whole frontal side of the body. Statistical analysis comparing each cohort with the ‘tumor only’ cohort at each time point was performed after log transformation using a repeated measures two-way ANOVA with Geisser-Greenhouse correction followed by Dunnett's multiple comparison test. Significance displayed for final BLI measurement at week 5; only TRAC-KI and CD247-KI show significantly reduced tumor burden in comparison to no treatment control, indicating a superior anti-tumor activity of CD247-KI over conventional lentiviral CAR T cells.

FIG. 19: CD247-KI HER2-CAR T display reduced exhaustion marker expression after in vitro expansion. Five HER2-specific CARs were cloned into the HDR templates for TRAC-KI or AAVS1-KI. The HER2-CARs differed in their respective scFv sequence with distinct affinity with Kd from 10e-11 to 10e-7 and shared the same generic 2^{nd} CAR design (IgG1-hinge, CD28 transmembrane, CD28 intracellular signaling domain and CD3 zeta). For CD247-KI, the truncated CD3-deficient HER2 CAR was cloned into the CD247 HDR template for exon 2. HER2-CAR T cells were generated via co-electroporation of Cas9-sgRNA RNPs for the respective target gene and the dsDNA HDR templates. After electroporation, CAR T cells were expanded in medium containing IL-2, IL-7 and IL-15 for additional 14 days and analysed for common exhaustion markers PD-1, TIM-3 and LAG-3 via flow cytometry. Spice analysis shows higher rate of exhaustion of CAR T cells when generated via knock-in into TRAC or hAAVS1 locus, however reduced exhaustion marker expressed after CD247-KI. The effect was seen with all four HER2 constructs (n=2).

FIG. 20: Reduced differentiation of CD247-KI HER2-CAR T cells after 2 week in vitro expansion. HER2-specific CAR T cells were expanded in medium containing IL-2, IL-7 and IL-15 for additional 14 days and analysed for their memory phenotype (CCR7, CD45RA) via flow cytometry. TRAC-KI and AAVS1-KI HER2 CAR T cells display higher proportion of cells with differentiated phenotypes (Tem, Temra). CD247-KI CAR T cells display highest proportion of cells with less-differentiated phenotype (Tnaive-like: Tn). (A) Representative flow plots are shown. (B) Summary is shown with bars indicate mean proportion (+/−standard deviation). (Temra: CCR7-negative CD45RA-positive; Tem: CCR7-negative and CD45RA-negative, Tcm: CCR7-positive and CD45RA-negative, Tn: CCR7-positive and CD45RA-positive)

FIG. 21: Reduced activation-induced cell death for CD247-KI Her2/neu-CARs after stimulation. Cryopreserved HER2-CAR T cells were thawed and stimulated via plate-bound anti-Fc antibody binding the CARs IgG1 hinge domains for 48 hours. The cells were stained for markers of apoptosis with 7AAD and Annexin V conjugated to Alexa Fluor 647 prior to flow cytometric analysis. Representative flow plots for HER2-CARs with highest affinity (Kd: 10e-11) and medium affinity (Kd: 10e-9) indicated severely reduced activation induced cell death of CD247-KI CAR T cells in comparison with CAR T cells generated via TRAC-KI orAAVS1-KI of expression cassettes driven by EF1alpha-LTR-promoter or CMV promoter.

FIG. 22: Intact killing capacity of primary NK cells (NKC) with CD3 zeta KO. Primary human NK cells show dose dependent Iysis of MHC-deficient K562 tumor cells, but also MHC-intact Raji lymphoma and Nalm-6 leukemia cell lines within a 4 hours co-culture experiment. Mock electroporated, CD3 zeta KO and TRAC KO control NK cells all conferred to comparable dose-dependent lysis of K562 cancer cells indicating that the CD3 zeta protein is not essential for missing-self recognition and missing self tumor killing. Mean+/−standard deviation, n=3.

FIG. 23: Zeta-KI CAR-NK cells have superior killing capacity than CAR-NK that overexpress the CAR from an ectopic locus (hAAVS1). The antigen-specific cytotoxicity of different NK92 cell lines was compared using a VITAL assay. Within the assay, CD19-positive GFP-positive Nalm-6 target cells and an equal amount of CD19-negative RFP-positive Nalm-6 control cells were co-cultured with different NK92 effector cell lines. Antigen-specific cytotoxicity mediated by different CAR-NKwas calculated from shifts in the target:control cell ratio relative to control conditions without effector cells. As expected, unmodified NK92 or NK92-reprogrammed with a mock-CAR did not induce antigen-specific lysis of CD19-expressing targets over CD19-negative control Nalm-6 cells. However, NK92 cells reprogrammed via CD247-knockin of a short/truncated CD19-specific CAR (NK92 zeta-KI AY, SEQ ID NO 59 and 60) had superior antigen-specific lysis of targeted cells in comparison to CAR-NK92 generated via AAVS1 knock-in of an expression cassette of the complete CD19-specific CAR (NK92 AAVS1 CMV CD19CAR-Y, SEQ ID NO 61 and 62) or another CD19-CAR (NK92 AAVS1-CMV CD19CAR-K, SEQ ID NO 63 and 64). Negative values were set to 0. Mean+/−standard deviation (n=6). Statistical analysis was performed with two-way-ANOVA between NK92 z-Y and all other effector cells and p-value calculated with Dunnett test. P values are <0.0001 for all tests (indiciated by ****).

### EXAMPLES

The invention is demonstrated through the examples disclosed herein. The examples provided represent particular embodiments and are not intended to limit the scope of the invention. The examples are to be considered as providing a non-limiting illustration and technical support for carrying out the invention.

The constructs employed in the examples are disclosed in the sequence tables above.

### Example 1: Novel Strategy Fuses Zeta-Deficient CAR Transgenes to Endogenous CD3 Zeta at Gene Level

In one embodiment, sketches in FIG. 1 present a comparison between CAR-T cells generated by conventional methods (FIG. 1A) and a preferred strategy provided by this invention (FIG. 1B). As shown in FIG. 1A, CD3-zeta protein expression from the CD247 gene. In the process of RNA transcription, CD247 exons are spliced together to form a mature CD247 messenger RNA, which serves as a template for protein translation by the ribosome. The formed CD3-zeta protein assembles with other proteins to form a fully functioning TCR/CD3 complex. The intracellular domain of the CD3-zeta protein within the TCR/CD3 complex is identical to the CD3-zeta domain of conventional Chimeric Antigen Receptors. FIG. 1B illustrates the strategy of a preferred embodiment of the invention. A truncated, CD3-zeta-deficient CAR transgene (preferably but not exclusively encoding a self-cleaving 2A-peptide, an antigen binding domain (e.g. an scFv), a hinge/spacer domain, a transmembrane domain and one or more co-stimulatory or regulatory domains but no Immunoreceptor Tyrosine-based Activation Motif (ITAM) containing signaling domain) is inserted into the CD247 gene. The modified CD247 gene is transcribed into mRNA encoding a full CAR with an endogenous CD3-zeta domain. During protein translation, the N-terminus of the expressed fusion protein is cleaved off the CAR protein via self-cleavage of the 2A peptide. While a functional CAR Protein is expressed on the surface of edited immune cells, the cleaved N-terminus of the CD3-zeta gene is non-functional and cannot serve for TCR/CD3 complex formation. As a result, successfully edited immune cells can express a CAR protein but are unable to express a TCR.

### Example 2: In-Frame Insertion of GFP into Different T Cell Receptor Complex Genes Leads to Loss of CD3-TCR Complex Surface Expression

In these experiments as shown in FIGS. 2 and 3, nuclease-assisted gene insertion of green fluorescent protein (GFP) reporter transgenes into the protein-coding reading frame of either the CD247 gene (encoding for CD3 zeta) or the TRAC gene (encoding for TCR alpha chain) was performed to investigate the effect on CD3-TCR surface expression. As indicated by flow cytometric protein analysis, most GFP expression was observed in the CD3-TCR negative population (Y-axis). Multiple Cas9 nucleases with different sgRNAs were used for CD247 exon 2 and 3 achieving similar results. Insertion of GFP into TRAC gene mirrored results seen in CD247, despite higher maximum GFP intensity.

Generation of dsDNA templates for homology-directed DNA repair: To this end, three different homology-directed repair (HDR) templates were designed to insert the transgene sequence in-frame into the exons 2 or 3 of the CD247 gene and the TRAC exon 1 via nuclease-assisted gene insertion. For TRAC locus, two templates were generated: one as previously published by Roth et al (PMID: 29995861), one self-designed template (TRAC exon 1 Charité) for use with the sgRNA TRAC sg1, which has fewer predicted off-target (PMID: 32241852). To allow for independent protein expression, the insert was designed to include a P2A-peptidase sequence and a bovine-growth hormone (bgh) poly-adenylation sequence. Synthesized homology arms (ca. 400 bp each) and inserts were cloned into pUC19 μlasmid backbone using In-Fusion system according to the manufacturer's protocols (Clontech, Takara). Plasmids were purified using column-based plasmid purification (Zymo Research, ZymoPure Miniprep Kit) and sequences were validated using Sanger Sequencing. For generation of highly-purified HDR templates made from double stranded DNA (dsDNA), polymerase chain reaction (PCR) with high-fidelity enzymes (KAPA HiFi HS Polymerase, 2× Readymix; Roche) was performed at large volumes (typically 500-1000 ul reactions) using 10-20 ng plasmid template. HDR templates were purified using Ampure XP magnetic bead purification (1 volume beads: 1 volume PCR product) according to the manufacturer's manual (Beckman Coulter). DsDNA HDR templates were resuspended in nuclease-free water; their purity was validated by agarose gel electrophoresis. HDR template concentration was measured using NanoDrop 1000 Spectrophotometer (ThermoFisher). Subsequently, HDR templates were aliquoted at 1000 ng/microliter or 2000 ng/microliter and cryopreserved at −20° C. until further use.

Nuclease formulation: CRISPR-Cas9 nuclease system was used to induce DNA double strand breaks and provoke DNA repair at the targeted locus. To this end, ribonucleoproteins (RNP) were formulated freshly for each experiment. Per 20 ul nucleofection volume, 0.5 μl poly-L-glutamic acid (15-50 kDa, 100 mg/mL, Sigma), 0.48 ul of respective sgRNA (100 uM, Synthego) and 0.4 μl of Sp.Cas9 (10 mg/ml, IDT) were mixed in this order and incubated for 15 minutes at room temperature in nuclease-free PCR tubes. Instantly prior to nucleofection, RNP were mixed with dsDNA HDR templates.

Cell culture and electroporation: Peripheral blood mononuclear cells (PBMCs) were isolated from heparinized venous blood of healthy human adults after informed consent. T cells were enriched using CD3+ microbeads and magnetic columns enrichment (MACS, Miltenyi Biotec). NK cells were enriched by using the human NK cell isolation kit and magnetic column depletion (MACS, LD columns, Miltenyi Biotec). NK92 cell line was procured via DSMZ-German Collection of Microorganisms and Cell Cultures GmbH. (DSMZ no: ACC 488). T cells were cultured in RPMI1640 (PAN) with 10% FCS (Biochrom) supplemented with IL-2 (100 IU/ml, Novartis), IL-7 (10 ng/ml, Cellgenix) and IL-15 (5 ng/ml, Cellgenix). NK cells were cultured in MACS NK cell expansion medium according to the manufacturer's instructions (Miltenyi Biotec) with 10% FCS (PAN) supplemented with IL-2 (200 IU/ml, Miltenyi) and IL-15 (5 ng/ml, Miltenyi). In some embodiments, NK cell expansion is aided by stimulation with NK Cell Activation/Expansion Kit, human (Miltenyi Biotec). NK-92 cells are cultured in RPMI 1640 (PAN) with 10% FCS supplemented with 200 IU/ml IL-2 (Miltenyi). 48 hours polyclonal stimulation on anti-CD3/28-coated tissue culture well plates, activated T cells were harvested and washed twice by resuspension in 1× phosphate buffered saline (PBS) and centrifugation at 100G for 10 minutes. Per 20 ul nucleofection, 1-1.5×10e6 T cells or 0.5-1.0×10e6 NK cells/NK-92 cells were used. Dried pellets were resuspended with nucleofection buffer P3 primary cell (Lonza) and mixed with RNP-HDR template suspension, and electroporated using the program EH-115 for T cells, DN-100 for primary NK cells and CA-137 for NK-92 cells (4D-Nucleofector, Lonza). Subsequently, nucleofected T cells are rescued with pre-warmed cytokine containing medium and transferred into tissue culture wells containing T cell medium supplemented with 15 μM HDR enhancer (IDT). For experiments with NK cells, no HDR enhancer was used. 16 hours post-electroporation, medium was changed to remove HDR enhancer. T and NK cells were split and cytokine-containing medium was replenished every other day.

Flow cytometric analysis: (2-) 7 (/14) days after nucleofection, T cells were harvested, washed with PBS, centrifuged at 400 G for 4 minutes. Supernatants were discarded and cell pellets resuspended prior to staining with LIVE/DEAD™ Fixable Blue Dead Cell Stain Kit (1:100, Molecular Probes, now ThermoFisher) and antibody for CAR detection (IgG-hinge containing constructs via anti-human Fc-gamma fragment antibody, 1:50, Jackson Laboratories; for other CARs via Myc-Tag, anti-Myc AF647, 1:50, Cell signaling) for 15 minutes at 4C in the dark. Then cells are washed and stained for other surface markers with CD3 APC-AF700 (1:100, Beckman Coulter) or CD3 PacBlue (1:50, Biolegend or Beckman Coulter). In some embodiments, additional surface markers are stained, such as CD4 (anti-human CD4 PE, 1:25, Beckman Coulter), CD8 (anti-human CD8 BV510, 1:100, Biolegend), CD45RA (anti-human CD45RA ECD, 1:50, Beckman Coulter), CCR7 (anti-human CCR7 Alexa Fluor 488, 1:50, Biolegend), CD56 (anti-human CD56 Alexa Fluor 647, Biolegend) for 15 minutes at 4C in the dark. For analysis of CD3 zeta in NK cells, cells were fixed and permeabilized with Fix/Perm (BD) prior to analysis. Subsequently, cells were washed again and resuspended for analysis on Cytoflex LX, flow cytometer. Data was analysed with FlowJo 10 software (BD). Representative plots were selected displaying alive single lymphocytes in the dimensions CD3 (TCR complex) in y-axis and GFP (successful gene insertion).

### Example 3: GFP Intensity is Lower when Expressed Under Control of the CD247 Promotor Compared to the TRAC Promotor

This subanalysis shown in FIG. 4 from the previous experiment (FIG. 3) compares GFP knock-in (KI) rates and fluorescence intensity in three replicates summarized for the preferred conditions (CD247: HDR template exon 2+ sgRNA 2.1; TRAC: exon 1 Charité+TRAC sg1). While relative KI did not differ significantly between TRAC and CD247, GFP expressed under control of the CD247 promotor leads to a lower median fluorescence intensity (MFI). [KI=knock-in]

### Example 4: In NK Cells, Transgenes can be Expressed Under Control of CD247 but not TRAC

CD3 zeta protein is expressed in NK cells at sufficient levels and is not essential for other NK cell functions, e.g., killing of tumor cells/missing-self recognition. (FIG. 14 A,B, and FIG. 15). In another experiment as shown in FIG. 5, nuclease assisted gene insertion was repeated in Natural Killer (NK) cells (modeled by NK92 cell line) using preferred HDR templates and sgRNA-nuclease as before (FIG. 4, CD247: HDR template exon 2+ sgRNA 2.1; TRAC: exon 1 Charité+TRAC sg 1). To this end, NK92 cells were expanded in RPM11640 medium supplemented with 10% FCS, 1% penicillin/streptomycin (Biochrom) and IL-2 (500 IU/ml). Four days prior to electroporation, NK92 were seeded in antibiotic free medium. In preparation, RNPs and HDRTs as well as cells were prepared as previously described (FIG. 3). Nucleofection was performed with program CA137. NK92 cells were subsequently cultured for 2 days. Flow cytometric analysis was performed as before, but without CD3 antibody. Here, representative flow cytometric analysis are shown and compared to results from experiments with primary T cells (FIG. 4). NK92 cell engineered with CD19 targeting CAR using the CD247 KI strategy (FIG. 14 C) and display superior antigen-specific cytotoxicity over CAR-NK92 generated via overexpression from the AAVS1 safe harbor locus (FIG. 23). Further, primary NK cells expand after CD3 zeta KO (FIG. 16).

### Example 5: Insertion of Complete and Zeta-Deficient CAR Transgenes into CD247 Gene Induce CAR Protein Surface Expression

Nuclease assisted gene insertion was used to introduce complete or zeta-deficient (truncated) CAR transgenes into the CD247 gene (FIG. 6). To perform this experiment, new HDR templates were designed. We rationally designed a 2nd generation CAR consisting of a murine CD19-specific single chain variable fragment (scFv), clone FMC63, an intermediate hinge CH3 spacer (derived from human Fc-IgG1), a CD28 transmembrane domain, a CD28 costimulatory intracellular domain and the CD3 cytoplasmic zeta chain. This CAR was cloned into HDR templates presented before (FIG. 3-5, Table 1). For CD247 HDR templates, a full CAR expression module with CD3 zeta and poly-A terminator sequence as well as a short CAR sequence lacking a poly-A terminator sequence were generated to test the hypothesis of this invention. Nucleofection was performed as described in FIG. 3. Flow cytometry was performed 7 days after nucleofection. To stain for CAR via its IgG1 hinge, cells were stained with LIVE/dead and anti-Fc Alexa Fluor 647 (Jackson) first. After washing, a second surface stain with anti-CD3 PacBlue (Biolegend) was done. (Note: for this experiment CD247 sg2.2 was used. For all subsequent experiment CD247 sg2.1 is used)

TABLE 1
sgRNA sequences and respective locations/donor templates.

| Location & | | | | | |
|---|---|---|---|---|---|
| corresponding | | | Ctrl: TRAC | Ctrl: TRAC | |
| donor template | CD247 | CD247 | Exon 1- | Exon 1- | Ctrl: |
| names: | Exon 2 | Exon 3 | Marson Lab | Charité | AAVS1 |
| SpCas9 sgRNAs | CD247 | CD247 sg1: | TRAC (Roth | TRAC sg 1 | AAVS1 (Mali |
| | sg2.1: | gacgcccccg | et al 2018): | GGGAAUC | et al 2013) |
| | gaUggaaUc | cgUaccagca | AGAGUCU | AAAAUCGG | GGGGCCA |
| | cUcUUcaUc | (SEQ ID NO | CUCAGCU | UGAAU | CUAGGGA |
| | Ua | 53) | GGUACA | (SEQ ID NO | CAGGAU |
| | (SEQ ID NO | | (SEQ ID NO | 55) | (SEQ ID NO |
| | 52) | | 54) | | 56 |
| | CD247 | | | | |
| | sg2.2: | | | | |
| | UGGUACC | | | | |
| | CACCUUCA | | | | |
| | CUCUC | | | | |
| | (SEQ ID NO | | | | |
| | 57) | | | | |
| | CD247 | | | | |
| | sg2.4: | | | | |
| | UgccUUgU | | | | |
| | UccUgagag | | | | |
| | Uga | | | | |
| | (SEQ ID NO | | | | |
| | 58) | | | | |

[sg = single guide RNA, AAVS1 = Adeno-associated virus integration site 1]]

### Example 6: Introducing Shorter Insert Size Tends to Increase Absolute CAR T Cell Generation after Non-Viral Gene Insertion

The effect of insert size (full CAR vs short zeta-less CARs, FIG. 7) on absolute CAR+ T cell counts was compared after nuclease assisted gene insertion with two different non-viral dsDNA HDR template variants. HDR templates were either generated with regular homology arms (HA) (FIG. 3, previously described in PMID: 29995861) or with homology arms modified with truncated Cas9 targeting sequences (tCTS) (16 bp, previously described in PMID: 31819258). After Electroporation, cells were either cultured in T cell medium or in T cell medium supplemented with 15 μM (0.5%) Alt-R HDR-Enhancer (Integrated DNA Technologies, Catalog #1081073). Two HDR template concentrations (0.75 or 1.50 μg/20 μl Electroporation) were compared. Regardless of the HDR template format used, smaller insert size due to the zeta-less CAR transgenes lead to improved CAR+ T cell recovery 7 days after transfection, thereby indicating an advantage for CAR T cell generation using this approach.

### Example 7: CAR Expression Level is Regulated Most Strictly Under Control of the CD247 Gene

In this experiment as presented by FIG. 8, CAR expression levels were compared between CAR T cells generated by different strategies. Representative flow cytometry plots and summary of median fluorescence intensity of CAR+ CD3− cells are shown. CAR T cells generated through insertion of truncated CAR transgenes into the 00247 exon 2 (sg 2.1) display the lowest MFI compared to TRAC-integrated CARs or other controls. For overexpression controls, the CAR presented in FIG. 6 was cloned into an expression cassette commonly used for generation of CAR T cells via lentiviruses or via transposase-transposon technology. It includes an EF1-alpha promoter and a BGH poly-A terminator sequence. The expression cassette was cloned into an HDR template for insertion into the human safe harbor locus hAAVS1. Additionally, the CAR was cloned into the SIN epHIV7 lentiviral vector plasmid for virus preparation and lentiviral transduction was performed as previously described (PMID: 22707919). [MFI=median fluorescence intensity]

Nalm-6 tumor cell rechallenge assay and exhaustion panel

Different CD19-CAR T cell conditions were seeded into a flat-bottom 96-well plate at a density of 25,000 CAR+ cells/well using X-vivo 15 medium supplemented with 10% FCS and 50 IU/ml IL-2. Every 12 hours, gamma-irradiated (30 Gy) GFP+ Nalm-6 were added to the CAR T cells in a 1:1 cellular ratio. 24 hours after the 4th round of co-culture, CAR T cells were analyzed by flow cytometry for expression of the inhibitory receptors PD-1 (anti-PD1 BV785, 1:25, Biolegend), LAG-3 (anti-human LAG3 PerCp-eFluor710, 1:25, eBioscience) and Tim-3 (anti-human Tim-3 APC-Cy7, 1:50, Biolegend).

After repeated tumor re-challenge, CD247-KI CAR T cells express less exhaustion markers, such as Tim-3, PD-1, and LAG-3, versus overexpression with a Lenti-virus or an AAVS1 (FIG. 17). Results from the TRAC-KI and CD247-KI strategy are comparable. Cell fitness of CAR-T cell engineered with CD247 KI approach improved (FIG. 17).

### Example 8: CD247 KI CAR T Cell Lyse CD19+ Target Cells in Antigen-Specific and Dose-Dependent Manner (as Well as at a Comparable Rate to TRAC/CD247-Integrated Full CARs)

CAR T cells generated either by short (truncated) or full CAR transgenes lysed target cells in an antigen- and dose-dependent manner and with similar efficacy as TRAC-integrated CAR T cells (FIG. 9). The only CAR T cell condition with reduced killing capacity at 2-1 and 1-1 T cell: target ratios was generated with the HDR template CD247 exon 3 inserting a full CAR expression cassette. To test the cytolytic activity of CAR T cells generated through transgene insertion into the CD247 locus, overnight co-culture assays with tumor cells were performed. CAR T cells generated either with nuclease assisted gene insertion into the CD247 exon 2, CD247 exon 3 or TRAC exon 1 were tested. CD19+ tumor cells (Nalm6) or CD19− tumor cells (Jurkat) were stained with CFSE to allow discrimination between targets (tumor cells) and effectors (CAR T cells). Co-cultures were performed in 96-U-Bottom-Well plates with 25.000 tumor cells per well. Depending on desired T cell: target ratio, respective numbers of CAR T cells were added to the specific wells. As controls, tumor cells were cultured without any T cells. On the following day, the cells were washed with PBS, centrifuged at 400 G for 10 minutes and stained with LIVE/Dead dye to exclude apoptotic cells. Subsequently, surviving cells were quantified by volume-based flow cytometric acquisition (Cytoflex LX, Beckman). The percentage of killing was calculated by normalizing to the negative control. TRAC-integrated CAR T cells served as a positive control. [KI=knock-in]

### Example 9: CD247 Short CAR KI T Cells Perform Comparable to Full CAR Knock-In in Serial Tumor Challenge

CAR T cells reprogrammed by insertion of truncated (zeta-deficient) CARs into the CD247 gene were able to eliminate tumor cells repeatedly (FIG. 10). At the day of assay setup, 25.000 T cells were mixed with 50.000 CD19+ tumor cells (Nalm6) in cytokine-free medium (RPMI1640+10% FCS). Per condition, four wells were prepared. The first well was analysed at setup day to control absolute tumor cell counts. Every third day, one well per condition was analysed and 50.000 CD19+ tumor cells (Nalm6) are added to the remaining wells. In summary, absolute tumor cell counts are presented. In negative controls (conditions with no T cells or with wildtype T cells), tumor cells grew uncontrolled. Both TRAC-integrated full-CAR equipped and CD247-integrated short CAR-reprogrammed T cells repeatedly eliminated tumor cells. [KI=knock-in]

### Example 10: Reduced Cytokine Production after Antigen-Engagement by Truncated CARs Integrated in the CD247 Gene Vs Full CAR in the TRAC Locus

As presented before (e.g. FIG. 8), CD247 integrated zeta-deficient CAR-reprogrammed T cells have a lower CAR expression level. While cytolytic activity remained unaffected by lower CAR surface density (FIG. 9,10), secretion of inflammatory cytokine TNF-alpha was reduced after challenge with CD19+ tumor cells (Nalm6) in both CD4+ and CD8+ T cells (FIG. 11). One hour after start of co-culture, brefeldin A (Sigma) was added at 10 μg/ml to prevent cytokine secretion into the medium. After 6 hour total incubation, the reaction was stopped by washing in cold PBS. Then, surface staining with LIVE/dead Blue Cell Dye (1:100, Thermo Fisher) was performed to exclude dead cells. Fixation and permeabilization was performed with Foxp3 Transcription Staining Kit according to the manufacturer's recommendations (Ebioscience, ThermoFisher). Subsequent intracellular staining was performed in two steps; first anti-Fc Alexa Fluor 647 (1:50, Jackson) was stained at 4° C. for 20 minutes. After wash with Permbuffer (same kit as before), a second intracellular staining was performed with antibodies for CD4, CD8, and TNF-alpha.

### Example 11: CD247 KI Abolishes Allo-Reactivity of CAR T Comparable to TRAC KI

As presented before (e.g. FIG. 6), insertion of (truncated) CARs into the TRAC as well as the CD247 gene eliminates the CD3-TCR from the T cell surface. To test whether this eliminates allogeneic cell killing, we performed co-culture assay with human T cells from two unmatched healthy donors with strong allo-reactivity against the CD19-negative cell line MM1S expressing a GFP reporter (FIG. 12). After four days, unmodified wildtype T cells eliminated a significant fraction of the tumor cells. In contrast the amount of tumor cells remained unchanged for TRAC-inserted CAR T cells or they increased in the presence of the CD247-integrated CAR T cells, indicating undetectable rate of allo-activity. [KI=knock-in]

### Example 12: Integration of Truncated CARs into CD247 Gene can be Used to Generate CAR+ Regulatory T Cells

CD4+ Treg were generated from PBMCs as described before (FIG. 3). On the day of PBMC isolation, CD8+ cells were eliminated using CD8+ microbead depletion (Miltenyi Biotec). The following day, CD25+ cells were enriched (CD25 microbeads, Miltenyi Biotec) from the CD8-negative cell fraction to isolate CD4+CD25+ T cells which contain >70% regulatory T cells. Subsequently, the cells activated with anti-CD3/28-coated beads and expanded in X-Vivo cell medium (Lonza) supplemented with 10% FCS, high doses of IL-2 (500 IU/ml) and Rapamycin (Treg medium) as described before (PMID: 33087345). On day 7 after PBMC isolation, regulatory T cells were re-stimulated with anti-CD3/28-coated beads. Next day, the beads were magnetically depleted and nucleofected as described before (FIG. 3). Rescue was performed with the Treg medium. After resting one more day, the nucleofected T cells were re-stimulated with irradiated CD19+ target cells (Nalm-6) and sub-cultured for additional 7 days prior to flow cytometric analysis.

### Example 13: CD247-KI Strategy Allows Reprogramming of Different Cell Types for Adoptive T Cell Transfer

TCR γ/δ T cells Treg cells, and NK cells can be reprogrammed by CD247-KI approach. Positive enrichment for TCR γ/δ was performed using Miltenyi MACS column purification (FIG. 14 A,B). Subsequently, cells were stimulated on plate-bound anti-CD3/CD28 and nucleofected with either CRISPR-Cas9 ribonucleoproteins and HDR donor templates for AAVS1 or CD247. Representative flow cytometry readout shows CAR expression in efficient editing of CD247 locus and CAR expression 4 days after electroporation. CD4 regulatory T cells can be reprogrammed with TRAC or CD247-KI (FIG. 13). CD4+ Treg were isolated in a two-step MACS selection process to isolate CD4+CD25+ T cells which contain >70% CD25+ Foxp3+ regulatory T cells. Subsequently, the cells activated with anti-CD3/28-coated beads and expanded in medium supplemented with high doses of IL-2 (500 IU/ml) and rapamycin. On day 7 after isolation and expansion, magnetic beads were depleted and regulatory T cells nucleofected. After resting for one day, the nucleofected T cells were re-stimulated with irradiated CD19+ target cells (Nalm-6) and sub-cultured for additional 7 days prior to flow cytometric analysis. NK cells could also be reprogrammed by CD247-KI approach (FIG. 14 C, FIG. 16). As a model for primary NK cells, the NK92 cell line was transfected with CRISPR-Cas9 ribonucleoproteins and dsDNA donors. While TRAC-KI does not induce GFP/CAR expression, CD247 gene targeting allows expression of transgenes (FIG. 5, FIG. 14 C). CD247-KI CAR enhances killing of Nalm-6 CD19+, but not CD19-negative control cells in a 6 hour VITAL assay (FIG. 14 D).

### Example 14: CD247-KI CAR T Cells Show Improved Functionality Overlentiviral CD19-CAR T Cells In Vitro and in a Leukemia Xenograft Mouse Model

Using sgRNA 2.1 and integration of a short/truncated CD19-CAR, we generated CD247-KI CAR T cells that displayed reduced frequency of exhaustion markers after four rounds of tumor stimulations in comparison with conventional lentivirus-transduced CD19-CAR T cells (FIG. 17). To validate whether this could also translate into superior anti-tumor activity in vivo, we compared their functionality in a systemic leukemia xenograft mouse model. To this end, 8 weeks old NRG mice were challenged i.v. with 5×10e5 GFP- and luciferase-expressing Nalm-6 leukemia cells per mouse. Four days after tumor cell infusion, 5×10e6 CAR-positive and TCR-negative CAR T cells were transplanted per mouse via tail vain injection. To avoid xenogenic GvHD in all conditions, lentiviral-transduced CAR T cells were electroporated with TRAC sgRNA 1 and Sp. Cas9 two days after viral spin transduction. To remove any contaminating TCR/CD3-positive cells, CD3 MACS depletion was performed for all conditions prior to CAR-T cell infusion (according to the manufacturer's instructions, Miltenyi Biotec). Tumor burden was analyzed weekly via bioluminsence using an IVIS device (method section). After five weeks, the mice were sacrificed without displaying any signs of GvHD or severe disease. Bioluminescence imaging revealed that within the first three weeks, all CAR T cell conditions slowed leukemia progression; however at week 5 only zeta-KI CD19-CAR T cells showed significantly reduced tumor burden than the no-CAR T cell control group, indicating superior anti-tumor activity (FIG. 18 A, B).

### Example 15: Zeta Approach is Transferable to Other CAR Targets (e.g. HER2)

Limited CAR T cell persistence and exhaustion-related dysfunction have been associated with reduced efficacy of CAR T cell therapy in different solid tumor indications. As the data with Zeta-KI CD19-CAR T cells indicated reduced capacity for exhaustion and improved anti-tumor activity (FIG. 17+18), we transferred the CD247-KI approach to another CAR platform relevant for different solid tumor indication. Therefore, we generated HDR templates to integrate several 2nd generation HER2 (a.k.a. HER2/neu, human epidermal growth factor receptor 2, ERB2)-specific CAR T cells that were previously described (Textor et al 2014; Cancer Res. 2014 Dec. 1; 74(23):6796-805. doi: 10.1158/0008-5472.CAN-14-0079 PMID: 25297631). All constructs harbored the same basic design, including an IgG1-hinge, a CD28 transmembrane, CD28 co-stimulatory domain and intracellular CD3 zeta chain, but differed in the respective Her2-specific scFvs that had distinct affinities from Kd values between 10e-11 to 10e-7. The CAR T cells were generated as before, with full CD3 zeta containing transgenes for TRAC-KI and AAVS1-KI conditions and short/truncated CD3 zeta-deficient CARs for the zeta-KI and sgRNA 2.1 (SEQ ID NO 52). After two weeks expansion, the CAR T cells were immunophenotyped for exhaustion markers (FIG. 19), their differentiation status (FIG. 20) and cryopreserved. Regardless of the scFv-affinity, CD247-KI HER2 CAR T cells displayed massively reduced expression of exhaustion markers on the cell surface after in vitro expansion in the absence of re-stimulation. Similarly, a higher proportion of CD247-KI HER2 CAR T cells retained a less-differentiated phenotype (CCR7-positive, CD45RA-positive) in comparison to eitherAAVS1-KI or TRAC-KI CAR T cells (FIG. 20). Later, cryopreserved T cells were thawed and re-stimulated by plate-bound anti-IgG antibodies to evaluate their propensity to undergo activation-induced cell death (FIG. 21). While many of AAVS1-KI and TRAC-KI CAR T cells stained positive with the early apoptotic marker Annexin V, most CD247-KI HER2 CAR T cells remained viable.

### Example 16: Intact Killing Capacity of Primary NK Cells (NK) without CD3 Zeta Protein

CD3 zeta harbors multiple ITAM domains and therefore is considered important in CAR-independent NK cell activation and functionality. Primary NK cells can lyse different cancer without MHC expression (such as K562 cells), but also MHC-intact cells, such as Raji lymphoma cells or Nalm-6 leukemia cells (FIG. 22 A). To determine whether the functionality of primary NK cells lacking free-soluble CD3 zeta would be compromised, CD3 zeta KO was performed via electroporation of Sp.Cas9 and CD247-specific sgRNA 2.1. Surprisingly, primary CD3 zeta KO NK cells expanded with and without bead stimulation were able to lyse K562 cells effectively in a dose-dependent manner and results were comparable with mock-electroporated or TRAC-edited NK cells (FIG. 22). Furthermore, zeta-KI CAR-NK92 show superior cytolytic activity compared to CAR-NK92 that overexpress the same CAR from an ectopic locus with a common expression cassette (FIG. 23: NK92 zeta-KI Y (CD247-exon2_CD19CAR-Y)—SEQ ID NO 59 and 60; NK92 AAVS1-KI Y (AAVS1_CMV_CD19CAR-Y)—SEQ ID NO 61 and 62; NK92 AAVS1-KI K (AAVS1_CMV_CD19CAR-K)—SEQ ID NO 63 and 64).

### REFERENCES

Elsallab, M., Levine, B. L., Wayne, A. S. & Abou-El-Enein, M. CAR T-cell product performance in haematological malignancies before and after marketing authorisation. Lancet Oncol. 21, e104-e116 (2020).
Gundry, M. C. et al. Highly Efficient Genome Editing of Murine and Human Hematopoietic Progenitor Cells by CRISPR/Cas9. Cell Rep. 17, 1453-1461 (2016).
Osborn, M. J. et al. Evaluation of TCR Gene Editing Achieved by TALENs, CRISPR/Cas9, and megaTAL Nucleases. Mol. Ther. 24, 570-581 (2016).
Qasim, W. et al. Molecular remission of infant B-ALL after infusion of universal TALEN gene-edited CAR T cells. Sci. Transl. Med. 9, (2017).
Eyquem, J. et al. Targeting a CAR to the TRAC locus with CRISPR/Cas9 enhances tumour rejection. Nature 543, 113-117 (2017).
MacLeod, D. T. et al. Integration of a CD19 CAR into the TCR Alpha Chain Locus Streamlines Production of Allogeneic Gene-Edited CAR T Cells. Mol. Ther. 25, 949-961 (2017).

1. A nucleic acid construct comprising:
a first nucleic acid sequence region, encoding a chimeric antigen receptor (CAR) fragment without a sequence encoding a functional intracellular domain of a CD3 zeta protein,
a second nucleic acid sequence region, comprising a targeting sequence configured for integrating the construct at an endogenous CD3 zeta gene of a human cell, and
a third nucleic acid sequence region encoding and/or comprising a protein separation site upstream of the first sequence region.

2. The nucleic acid construct according to claim 1, wherein the first nucleic acid sequence region encoding a CAR fragment comprises:
a nucleic acid sequence encoding an extracellular antigen-binding domain,
a nucleic acid sequence encoding a transmembrane domain, and
a nucleic acid sequence encoding one or more intracellular co-stimulatory domains.

3. The nucleic acid construct according to claim 1, wherein the targeting sequence is configured for integrating the construct in-frame with an endogenous CD3 zeta encoding sequence.

4. The nucleic acid construct according to claim 1, wherein the protein separation site of the third nucleic acid sequence encodes a first self-cleavage peptide, protease cleavage site or an internal ribosomal entry site (IRES).

5. The nucleic acid construct according to claim 1, wherein the target site of the CD3 zeta gene, into which the first sequence region is integrated, is an exon and/or an intron, and is positioned upstream of an endogenous CD3 zeta sequence encoding an intracellular domain or fragment thereof, said intracellular domain or fragment thereof comprising one or more immunoreceptor tyrosine-based activation motifs (ITAMs).

6. The nucleic acid construct according to claim 1, wherein the first nucleic acid sequence region encoding a CAR fragment additionally comprises a sequence encoding a CAR-regulating, immune-stimulating and/or therapeutic protein, in-frame and separated from the CAR fragment-encoding region by a protein separation site.

7. The nucleic acid construct according to claim 1, wherein the targeting sequence of the second sequence region is configured for integration into a host genome by a gene editing technique,
and/or
wherein the targeting sequence comprises an identical sequence to a recognition site of the endogenous DNA sequence into which integration is intended.

8. A genetically modified human cell, comprising an exogenous nucleic acid sequence region encoding a CAR fragment, without a sequence encoding a functional intracellular domain of a CD3 zeta protein, integrated in-frame into an endogenous CD3 zeta gene.

9. The genetically modified cell according to claim 8, wherein a CAR is expressed as an in-frame fusion protein comprising the CAR fragment and an intracellular domain of the endogenous CD3 zeta gene, said intracellular domain comprising one or more ITAMs.

10. The genetically modified cell according to claim 8, wherein the CAR-CD3 zeta fusion gene is expressed under the control of an endogenous CD3 zeta promoter.

11. The genetically modified cell according to claim 8, wherein the endogenous expression and/or function of a TCR complex is disrupted.

12. The genetically modified cell according to claim 8, wherein the cell is an immune cell, or a cell derived from peripheral human blood, human cord blood, bone marrow stem cells or induced pluripotent stem cells (iPSC).

13. A method for the treatment of a medical disorder associated with the presence of pathogenic cells expressing oncogenes or for the treatment of an autoimmune disease or a graft versus host disease, the method comprising administering a genetically modified cell according to claim 8 to a subject in need thereof.

14. The method according to claim 13, wherein the genetically modified cell is allogeneic with respect to a patient.

15. The method according to claim 13, wherein the genetically modified cell is autologous with respect to a patient.

16. A CAR polypeptide expressed from a genetically modified cell according to claim 8, comprising a CAR as an in-frame fusion protein comprising an exogenous CAR fragment without a sequence encoding a functional intracellular domain of a CD3 zeta protein, and an intracellular domain of the endogenous CD3 zeta gene, said intracellular domain comprising one or more ITAMs.

17. A pharmaceutical composition comprising a genetically modified cell according to claim 8 and a pharmaceutically acceptable carrier.

18. The nucleic acid construct according to claim 3, wherein the targeting sequence is configured for integrating the construct in-frame into an exon of an endogenous CD3 zeta gene.

19. The nucleic acid construct according to claim 6, wherein the protein separation site is a sequence encoding a second self-cleavage peptide, a promoter, and/or an IRES site.

20. The nucleic acid construct according to claim 7, wherein the gene editing technique is CRISPR-Cas, zinc finger nucleases (ZFNs), integrases, site specific recombinases, meganucleases, homing endonucleases, or TALENs, and the recognition site of the endogenous DNA sequence into which integration is intended is selected from the group consisting of a homology arm, a guide RNA target site, a restriction enzyme recognition site, a ZFN recognition site, a ZFN cleaving site, a TALEN DNA-binding site, a recombinase recognition site, an integrase site and/or a homing nuclease recognition site.

21. The genetically modified cell according to claim 10, wherein expression of the CAR is terminated by an endogenous CD3 zeta gene expression termination signal and an endogenous poly-A signal.

22. The genetically modified cell according to claim 12, wherein the immune cell is selected from the group consisting of a T lymphocyte, a T regulatory (Treg) cell, natural killer (NK) cell, a natural killer T (NKT) cell, cytokine-induced killer cell (CIK), an immortalized immune cell including NKT, NK-92 and YT cells.

23. The genetically modified cell according to claim 22, wherein the T lymphocyte is a CD4 or CD8 T-cell.

24. The genetically modified cell according to claim 23, wherein the T lymphocyte is a cytotoxic T lymphocyte or a T helper cell.
